(19) United States
(12) Patent Application Publication
(10) Pub. No.: US 2024/0263233 A1
(43) Pub. Date: Aug. 8, 2024

(54) Systems for Processing a Cellular Sample, Multi-Microwell Array-Flowcell Cartridges for Use Therein, and Methods of Use Thereof

(71) Applicant: Becton, Dickinson and Company, Franklin Lakes, NJ (US)

(72) Inventors: Raymond Brasil Cota, San Jose, CA (US); Jamie Ryan Moskwa, San Jose, CA (US); Germin Hanna Zaki, Sunnyvale, CA (US); Jrhung Taylor Tsai, Alameda, CA (US); Yasaman Daghighi, Oakland, CA (US); Timothy Zheng Zhu, San Jose, CA (US)

(21) Appl. No.: 18/427,553

(22) Filed: Jan. 30, 2024

Related U.S. Application Data
(60) Provisional application No. 63/443,326, filed on Feb. 3, 2023.
(60) Provisional application No. 63/468,622, filed on May 24, 2023.

(51) Int. Cl. C12Q 1/6876; B01L 3/00; C12Q 1/6806; C12Q 1/6874; G01N 33/68

(52) U.S. Cl. CPC C12Q 1/6876; B01L 3/5025; B01L 3/502761; C12Q 1/6874; G01N 33/6803; B01L 2200/04; B01L 2200/0668; B01L 2200/16; B01L 2300/087; B01L 2400/043; C12Q 1/6806; C12Q 2600/16; G01N 2570/00

(57) ABSTRACT

Systems for processing a cellular sample are provided. Embodiments of the systems include a tray configured to receive a multi-microwell-array-flowcell cartridge, and a retrieval magnet assembly configured to apply a uniform magnetic force to flowcells of the cartridge when in an active position. Also provided are methods, which methods may include introducing a multi-microwell-array-flowcell cartridge into the system of the invention, loading a sample into the multi-microwell-array-flowcell cartridge, actuating the retrieval magnet assembly to the active position and applying the uniform magnetic force to the sample within the multi-microwell-array-flowcell cartridge to produce a processed sample, and collecting the processed sample from the multi-microwell-array-flowcell cartridge. In addition, multi-microwell-array-flowcell cartridges are disclosed. Embodiments of the subject cartridges include a plurality of fluidic lanes, each fluidic lane comprising, an inlet for receiving liquid, a flow cell comprising a microwell array, and an outlet for discharging liquid.

### CROSS-REFERENCE TO RELATED APPLICATIONS

Pursuant to 35 U.S.C. § 119 (e), this application claims priority to the filing date of U.S. Provisional Patent Application Ser. No. 63/468,622 filed May 24, 2023, as well as to U.S. provisional application Ser. No. 63/443,326 filed on Feb. 3, 2023, the disclosure of which is herein incorporated by reference.

### INTRODUCTION

The ability to detect and quantify specific nucleic acid and protein molecules in individual cells is critical for understanding the role of cellular diversity in development, health, and disease. Flow cytometry has become a standard technology for high-throughput detection of protein markers on single cells and has been widely adopted in basic research and clinical diagnostics. In contrast, nucleic acid measurements such as mRNA expression are typically conducted on bulk samples, obscuring the contributions from individual cells.

Methods and techniques such as stochastic barcoding are useful for cell analysis. For example, stochastic barcoding can be used to decipher cell physiological conditions, for example protein and/or gene expression profiles of single cells, to determine their states using, for example, reverse transcription, polymerase chain reaction (PCR) amplification, and next generation sequencing (NGS). However, a detected expression profile may be associated with two or more cells of different types, which can skew the interpretation of the expression profile.

Array technologies have been used in biomedical studies. Arrays are equipped with probes which can be hybridized with target molecules with labels, e.g., fluorescence. A feature on an array is a small cluster of the same or similar probes with specific molecular sequences, say, DNA or RNA. Identifying the label patterns on a hybridized array can infer the hybridization taking place in the sample, which in turn can further assist biomedical studies. Loading stations for the loading and retrieval of particles in array devices have been described in U.S. Pat. Nos. 10,634,691 and 11,061,043; the disclosures of which are incorporated by reference herein in their entirety.

In order to characterize the complexity of cellular systems, it is highly desirable to develop methods, devices, and systems for monitoring the expression of large numbers of genes across many thousands of cells. Current technology allows measurement of gene expression of single cells in a massively parallel manner (e.g., >10000 cells) by attaching cell specific oligonucleotide barcodes to poly(A) mRNA molecules from individual cells as each of the cells is co-localized with a barcoded reagent bead in a compartment.

### SUMMARY

The inventors have realized that there is a continued need for improved systems and methods for preparing sequence-ready nucleic acid libraries from single cells. For example, while the loading stations described in U.S. Pat. Nos. 10,634,691 and 11,061,043 have improved the process for preparing cellular samples, it has been discovered that additional enhancements are desirable. Embodiments of the present invention satisfy these and other desires.

Aspects of the invention include systems for processing a cellular sample. Systems of embodiments of the invention include a tray configured to receive a multi-microwell-array-flowcell cartridge, and a retrieval magnet assembly configured to apply a uniform magnetic force to flowcells of the cartridge when in an active position. In certain cases, the retrieval magnet assembly is configured to apply the uniform magnetic force from a position that is superior to the tray. In some cases, the retrieval magnet assembly is configured to apply the uniform magnetic force from a position that is superior to the tray. The uniform magnetic force may, in certain cases, be a magnetic field ranging from 650 Gauss to 1325 Gauss. In certain instances, the retrieval magnet assembly is configured to apply the uniform magnetic force via a plurality of magnets. In some such instances, the plurality of magnets have alternating polarities. The number of magnets in the retrieval magnet assembly may vary, and in some cases range from 2 to 10 (e.g., 4). The type of magnets in the retrieval magnet assembly may also vary. In some instances, the magnets are rare earth magnets (e.g., neodymium magnets and/or samarium-cobalt magnets). The shape of the magnets may also vary. In some cases, the magnets of the plurality are bar magnets. In other cases, the magnets of the plurality are ring magnets. In some such cases, magnets of the plurality are arranged in a bullseye configuration. Systems of the invention may be configured to adjust the position of the retrieval magnet assembly to process the cellular sample. In some embodiments, the retrieval magnet assembly is actuatable between the active position in which the retrieval magnet assembly is positioned adjacent to the tray, and an inactive position in which the retrieval magnet is located at a further distance from the tray relative to the active position.

In some versions, systems include a sample collection vessel holder configured to receive a plurality of sample collection vessels for collecting an analyte from the cartridge. In some such versions, the sample collection vessel holder comprises a counterweight configured to maintain the sample collection holder in an upright position. The system and the sample collection vessel holder may also have complementary shapes such that the sample collection vessel holder may be received in the system in a single orientation. In some aspects, systems also include the plurality sample collection vessels. The number of sample collection vessels may vary, and can range in some cases from 2 to 10 (e.g., 8). In certain instances, the system comprises a number of sample collection vessels that matches the number of flowcells in the cartridge. In some embodiments, a waste collection vessel for collecting liquid waste from the multi-microwell-array-flowcell cartridge. In some instances, systems include an interlock configured to prevent the collection of sample liquid into the waste collection vessel when the retrieval magnet assembly is in the active position. In some cases, the tray comprises a latch for retaining the multi-microwell-array-flowcell cartridge. In select versions, systems do not include a lysis magnet at an inferior position to the tray.

In some instances, systems additionally include the multi-microwell-array-flowcell cartridge. The multi-microwell-array-flowcell cartridge of interest includes a plurality of fluidic lanes, each fluidic lane comprising an inlet for receiving liquid, a flow cell comprising a microwell array, and an outlet for discharging liquid. In certain cases, the multi-microwell-array-flowcell cartridge comprises a number of fluidic lanes ranging from 2 to 10 (e.g., 8). In some versions, each outlet is stepped to prevent siphoning of liquid from the flow cell. In some embodiments, each flow cell is comprised of an elongate channel. The length of the elongate channel may vary, and can range in some cases from 50 mm to 100 mm. The number of microwells in each microwell array may also vary, and can range, in some cases, from 250,000 microwells to 300,000 microwells.

The density of microwells within each microwell may vary, and can range in some cases from 36,000 microwells/cm² to 42,000 microwells/cm².

In some cases, the tray has a shape that is complementary to the shape of the multi-microwell-array-flowcell cartridge such that the multi-microwell-array-flowcell cartridge may be received in the tray in a single orientation. In some such cases, the multi-microwell-array-flowcell cartridge comprises a chamfered corner. In embodiments, systems additionally include a drip receptacle located at an inferior position to the tray configured to contain liquid discharged from the cartridge.

Aspects of the invention also include methods of processing a cellular sample.

Methods of interest include introducing a multi-microwell-array-flowcell cartridge (e.g., as described above) into a system of the invention (e.g., as described above). Systems of interest for the subject methods include a tray configured to receive the multi-microwell-array-flowcell cartridge, and a retrieval magnet assembly configured to apply a uniform magnetic force to the flowcells of the multi-microwell-array-flowcell cartridge when in an active position. Methods also include loading a sample into the multi-microwell-array-flowcell cartridge, actuating the retrieval magnet assembly to the active position and applying the uniform magnetic force to the sample within the multi-microwell-array-flowcell cartridge to produce a processed sample collecting the processed sample from the multi-microwell-array-flowcell cartridge.

In certain cases, the method comprises loading a plurality of different samples into the different flowcells of the cartridge. The number of samples may vary and can range from, in some cases, from 2 to 10 (e.g., 8). Methods according to certain embodiments include loading a lysis buffer into the multi-well cartridge after the loading of the sample liquid into the multi-well cartridge. In some versions, methods include loading the lysis buffer into the multi-well cartridge without engaging a lysis magnet. Methods according to select versions of the invention include loading barcoded beads into the multi-well cartridge prior to the actuation of the retrieval magnet assembly. The barcoded beads may, in certain cases, include a nucleic acid barcode comprising a universal primer binding domain, a cell label domain and a target capture domain. In select embodiments, the target capture domain is an poly(T) sequence. In certain instances, the nucleic acid barcode further comprises a unique molecular index (UMI).

The type of analysis performed in the subject methods may vary, as desired. In some cases, the method comprises producing a sequence-ready nucleic acid library from the processed sample. In certain embodiments, the sequence ready nucleic acid library is sequence-able by using a next generation sequencing protocol. In additional embodiments, the method is a method of genomic analysis, epigenomic analysis, transcriptomic analysis or proteomic analysis. In select versions, the method is a method of multiomic analysis, such as where the multiomic analysis comprises at least transcriptomic and proteomic analysis.

Aspects of the invention also include multi-microwell-array-flowcell cartridges. Cartridges of interest include a plurality of fluidic lanes, each fluidic lane comprising an inlet for receiving liquid, a flow cell comprising a microwell array, and an outlet for discharging liquid. The disclosed multi-microwell-array-flowcell cartridges are configured for use in the subject systems and methods. In addition, aspects of the invention include a sample vessel holder configured to receive a plurality of sample collection vessels for collecting an analyte from the multi-microwell-array-flowcell cartridge of the invention. In some cases, the sample collection vessel holder has a complementary shape to the cellular analysis system such that the sample collection vessel holder may be received in the cellular analysis system in a single orientation.

Aspects of the invention may additionally include kits. Kits of the invention include a multi-microwell-array-flowcell cartridge of the invention. As discussed above, the multi-microwell-array-flowcell cartridge of the invention comprises a plurality of fluidic lanes, each fluidic lane comprising an inlet for receiving liquid, a flow cell comprising a microwell array, and an outlet for discharging liquid. Kits may additionally include one or more instruments for performing methods of the invention. For example, kits according to some embodiments include a sample collection vessel holder, one or more sample collection vessels, and one or more waste collection vessels. In some cases, kits also include one or more reagents for performing methods of the invention. For example, kits according to some embodiments may include a hybridization buffer, a wash buffer, a reducing agent, and barcoded beads.

### BRIEF DESCRIPTION OF THE FIGURES

The patent or application file contains at least one drawing executed in color. Copies of this patent or patent application publication with color drawing(s) will be provided by the Office upon request and payment of the necessary fee.

The invention may be best understood from the following detailed description when read in conjunction with the accompanying drawings. Included in the drawings are the following figures:

FIG. 1A-1E depict a retrieval magnet assembly and a tray according to certain embodiments, as well as the actuation of the retrieval magnet assembly from the inactive position (FIG. 1A-1B) to the active position (FIG. 1C-1D).

FIG. 2A-2B depict a retrieval magnet assembly according to certain embodiments.

FIG. 3A-3H depict systems for processing a cellular sample according to certain embodiments.

FIG. 4A-4B depict a trip tray according to certain embodiments.

FIG. 5A-5B depict an interlock according to certain embodiments.

FIG. 6A-6C presents a multi-microwell-array-flowcell cartridge according to certain embodiments.

FIG. 7A-7H present different views of a multi-microwell-array-flowcell cartridge according to certain embodiments.

FIG. 8 depicts alignment features of a multi-microwell-array-flowcell cartridge according to certain embodiments.

FIG. 9A-9C present different depictions of microwells of flowcells within cartridges of the invention.

FIG. 10 depicts fluid flow through a multi-microwell-array-flowcell cartridge according to certain embodiments.

FIG. 11A-11H depict cartridge carriers according to certain embodiments of the invention.

FIG. 12A-12C depict a sample collection vessel holder according to certain embodiments.

FIG. 13 presents a flowchart for practicing a method of the invention according to certain embodiments.

FIG. 14A presents a flowchart for practicing a method of the invention according to certain embodiments.

FIG. 14B is a schematic illustration showing a non-limiting exemplary process of generating an indexed library of the barcoded targets (e.g., stochastically barcoded targets), such as barcoded mRNAs or fragments thereof.

FIG. 15A-15J present experimental data regarding the magnetic flux density of magnets in a retrieval magnet assembly.

FIG. 16A-16B present data showing the higher throughput of the BD Rhapsody™ HT-Xpress.

FIG. 17A-17D present data showing flexible cell throughput per lane with the BD Rhapsody™ HT-Xpress.

FIG. 18A-18C present data showing flexible throughput with partial use of the BD Rhapsody™ HT-Xpress 8-lane cartridge.

FIG. 19A-19D present data showing flexible throughput with partial use of the BD Rhapsody™ HT-Xpress 8-lane cartridge.

FIG. 20A-20B present data showing the capture of cells of different sizes with BD Rhapsody™ HT-Xpress.

FIG. 21A-21B present data showing the capture of fragile cells with the BD Rhapsody™ HT-Xpress.

FIG. 22A-22D present data related to two-step staining.

FIG. 23A-23C show antigen specific T-cell profiling using BD Rhapsody™ Single-Cell Analysis. FIG. 23C includes, from top to bottom, SEQ ID NOs: 3-13.

FIG. 24A-24L show simultaneous profiling of mRNA, surface and intracellular proteins.

FIG. 25A-25C demonstrate that there is no batch effect between technical replicates.

### DETAILED DESCRIPTION

Systems for processing a cellular sample are provided. Systems of embodiments of the invention include a tray configured to receive a multi-microwell-array-flowcell cartridge, and a retrieval magnet assembly configured to apply a uniform magnetic force to flowcells of the cartridge when in an active position. Methods of practicing the invention are also provided, where embodiments include introducing a multi-microwell-array-flowcell cartridge into the system of the invention, loading a sample into the multi-microwell-array-flowcell cartridge, actuating the retrieval magnet assembly to the active position and applying the uniform magnetic force to the sample within the multi-microwell-array-flowcell cartridge to produce a processed sample, and collecting the processed sample from the multi-microwell-array-flowcell cartridge. In addition, multi-microwell-array-flowcell cartridges are disclosed. The subject cartridges include a plurality of fluidic lanes, each fluidic lane comprising, an inlet for receiving liquid, a flow cell comprising a microwell array, and an outlet for discharging liquid.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise.

It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

While the apparatus and method has or will be described for the sake of grammatical fluidity with functional explanations, it is to be expressly understood that the claims, unless expressly formulated under 35 U.S.C. § 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 U.S.C. § 112 are to be accorded full statutory equivalents under 35 U.S.C. § 112.

### Systems for Processing a Cellular Sample

As discussed above, aspects of the invention include systems for processing a cellular sample. In some embodiments, systems include structures or features designed to facilitate performance of a workflow. In some embodiments, a workflow can include a series of steps for hybridizing nucleic acids from a plurality of cells with nucleic acids. e.g., barcoded nucleic acids, displayed on surfaces of a plurality of barcode-bearing beads. For example, in some embodiments, a workflow can include steps of introducing a plurality of cells into a microwell array, introducing a plurality of barcoded nucleic acid-bearing beads into the microwell array, lysing the plurality of cells so that nucleic acids from the cells, e.g., mRNA from the cells, hybridizes with barcoded nucleic acids associated with the plurality of beads (e.g., present on a surface of the beads), and then collecting the beads from the microwell array. In some instances, the workflow is a component of next generation sequence (NGS) library preparation workflow.

As noted above, systems of the invention include a tray configured to receive a multi-microwell-array-flowcell cartridge, and a retrieval magnet assembly configured to apply a uniform magnetic force to flowcells of the cartridge when in an active position. By “uniform magnetic force”, it is meant that the magnetic force is applied evenly over the area of the multi-microwell-array-flowcell cartridge to which it is applied. In other words, the magnetic force applied to one portion of the multi-microwell-array-flowcell cartridge differs minimally in comparison to the magnetic force applied to another portion of the multi-microwell-array-flowcell cartridge, such as by 20% or less, such as by 15% or less, such as by 10% or less, such as by 5% or less, and including by 1% or less. In embodiments, the uniform magnetic force of the invention results in comparable (including the same) amounts of magnetic force applied to each flowcell of the multi-microwell-array-flowcell cartridge when said cartridge is placed in the system, such as where the amount of magnetic force being applied to any two given flowcells differs by 20% or less, such as by 15% or less, such as by 10% or less, such as by 5% or less, and including by 1% or less. The uniform magnetic force may vary in strength. For example, in some cases, the uniform magnetic force is a magnetic field ranging from 650 Gauss to 1,325 Gauss, such as 650 Gauss to 700 Gauss, such as 700 Gauss to 750 Gauss, such as 750 Gauss to 800 Gauss, such as 800 Gauss to 850 Gauss, such as 850 Gauss to 900 Gauss, such as 900 Gauss to 950 Gauss, such as 950 Gauss to 1,000 Gauss, such as 1,000 Gauss to 1,050 Gauss, such as 1,050 Gauss to 1,200 Gauss, such as 1,200 Gauss to 1,250 Gauss, such as 1,250 Gauss to 1,300 Gauss, and including 1,300 Gauss to 1,325 Gauss.

In embodiments, the tray is positioned on a superior surface of a body of the subject system. In some embodiments, the tray can receive a multi-microwell-array-flowcell cartridge within the system. In some embodiments, the tray can be dimensioned, positioned, or otherwise configured to orient the multi-microwell-array-flowcell cartridge in a predefined position within the system. The predefined position can facilitate the interaction of other features of the loading station with multi-microwell-array-flowcell cartridge. In some embodiments, the tray can releasably secure the cartridge within the system. For example, in some embodiments, the tray has a shape that is complementary to the shape of the multi-microwell-array-flowcell cartridge such that the multi-microwell-array-flowcell cartridge may be received in the tray in a single orientation. For example, in some cases where the multi-microwell-array-flowcell cartridge includes one or more chamfered corners (described in greater detail below), the tray has a corresponding shape, i.e., it is configured to receive a cartridge having a chamfered corner. In this manner, the chamfered corner may be used as an orientation feature for cartridge installation.

In certain cases, systems include one or more retention mechanisms configured to maintain the multi-microwell-array-flowcell cartridge in place when said cartridge is placed in the tray. In some such cases, the tray includes a latch for retaining the multi-microwell-array-flowcell cartridge. In select embodiments, the latch is spring-loaded. The spring-loaded latch may be pushed back when the cartridge is inserted, and exert a force on the cartridge that constrains the cartridge in place. In some cases, the latch includes one or more protruding features allowing a user to slide the latch backwards when removing the cartridge. The latch may be constructed using any convenient technique. In some embodiments, the latch is injection-molded. In some such embodiments, the latch includes an injection-moldable polymer. Any convenient injection-moldable polymer may be employed. Injection-moldable polymers may include, but are not limited to: acrylonitrile butadiene styrene (ABS), polycarbonate (PC), aliphatic polyamides (PPA), polyoxymethylene (POM), polymethyl methacrylate (PMMA), polypropylene (PP), polybutylene terephthalate (PBT), polyphenylsulfone (PPSU), polyether ether ketone (PEEK) and polyetherimide (PEI). Where the latch is spring-loaded, the springs may in some cases be made of stainless steel (e.g., 18-8 stainless steel) to protect against oxidation due to nearby liquid. In some instances, latches additionally include a latch plate configured for anchoring the latch to the system. For example, in some cases, the latch plate is configured to anchor the latch to the system via one or more screws. Where present, the latch plate may be constructed from any suitable material. In some instances, the latch plate is constructed from a metal material, including but not limited to aluminum, titanium, brass, iron, lead, nickel, steel (e.g., stainless steel), copper, tin as well as combinations and alloys thereof. In embodiments, the latch plate is made of aluminum, such as 6061 aluminum.

In embodiments, trays also include a cartridge catch. The cartridge catch may be located on the opposite side of the tray relative to the latch, and provide a downward force to the cartridge when such is placed within the tray. Said downward force may be sufficient to prevent the cartridge from being removed from the tray until the latch is disengaged. The cartridge catch may be anchored to the system in any convenient manner, such as by one or more screws. Where present, the cartridge catch may be constructed from any suitable material. In some instances, the cartridge catch is constructed from a metal material, including but not limited to aluminum, titanium, brass, iron, lead, nickel, steel (e.g., stainless steel), copper, tin as well as combinations and alloys thereof. In embodiments, the cartridge catch is made of aluminum, such as 6061 aluminum.

Systems of interest also include a retrieval magnet assembly. As discussed above, the retrieval magnet assembly is configured to apply a uniform magnetic force to flowcells of the cartridge when in an active position. The retrieval magnet assembly may be employed, e.g., when it is desirable to retrieve beads in microwells of the cartridge. The retrieval magnet assembly is switchable between an active position and an inactive position. The active position is a position in which the retrieval magnet assembly applies the magnetic force to the flowcells of the cartridge, while the inactive position is a position in which the retrieval magnet assembly applies less magnetic force to the flowcells of the cartridge as compared to the active position (including no magnetic force). In the inactive position, the retrieval magnet assembly is positioned at a further distance from the multi-microwell-array-flowcell cartridge in at least one direction in comparison to the active position. In some cases, the retrieval magnet assembly is configured to apply the uniform magnetic force to the multi-microwell-array-flowcell cartridge from a position that is superior to the tray. In some embodiments, when in the active position, an inferior surface of the retrieval magnet assembly can be in parallel with a superior surface of the multi-microwell-array-flowcell cartridge. In select cases, the inferior surface of the retrieval magnet assembly can be 1.0 mm away or approximately 1.0 mm away from the superior surface of the multi-microwell-array-flowcell cartridge when in the active position. In select instances, the inferior surface of the retrieval magnet assembly can be 0.5 mm, 1.5 mm, 2.0 mm, 2.5 mm, 3.0 mm, less than 0.5 mm, less than 1.0 mm, no more than 1.0 mm, less than 1.5 mm, less than 2.0 mm, less than 2.5 mm, less than 3.0 mm, or between 0.5 mm and 1.5 mm away from the superior surface of the multi-microwell-array-flowcell cartridge. In some embodiments, systems can include a retrieval magnet assembly actuator. In some embodiments, the actuator can be actuated to transition the retrieval magnet assembly between the inactive position and the active position.

Although two positions of the retrieval magnet assembly are described above, it should be recognized that in certain embodiments, the retrieval magnet assembly can move over more than two positions. In some embodiments, the retrieval magnet assembly can move between discrete positions. In some embodiments, the retrieval magnet assembly can move over a continuous range of positions. In embodiments, positions of the retrieval magnet assembly can allow for the application of different amounts of magnetic force on magnetic particles within the multi-microwell-array-flowcell cartridge when the said cartridge is positioned within the tray.

In some embodiments, the system does not include a lysis magnet at an inferior position to the tray. A “lysis magnet” is a magnet is a magnet that is engaged during a lysis step in a method of cellular sample preparation. For example, the lysis magnet may pull and hold the beads into the microwells while the lysis buffer is pipetted through the flowcell. For example, magnet 120 disclosed in U.S. Pat. Nos. 10,634,691 and 11,061,043 is considered herein to be a lysis magnet that is not present in embodiments of the subject systems. Prior to the filing of the present disclosure, it was believed that a lysis magnet was required to prevent beads from being washed away. However, the present inventors made the surprising realization that the lysis magnet was able to be removed from the design and the beads would remain in the microwells during the application of the lysis buffer.

In some cases, the retrieval magnet assembly is configured to apply the uniform magnetic force via a plurality of magnets. In such cases, the number of magnets in the plurality of magnets may vary, and can range from 2 to 10, such as 2 to 6, and including 3 to 5. For example, the number of magnets may be 2, 3, 4, 5, 6, 7, 8, 9 or 10. In some instances, the retrieval magnet assembly comprises 4 magnets. The magnets in the retrieval magnet assembly may be comprised of any suitable magnet. Magnets of interest include, but are not limited to: rare earth magnets such as neodymium magnets, samarium-cobalt magnets as well as combinations thereof. The size and shape of the magnets may also vary. In some cases, the magnets are bar magnets. Where the magnets are bar magnets, the length of each bar magnet may vary, and can range in some instances from 50 mm to 500 mm, such as 60 mm to 200 mm, such as 70 mm to 80 mm, such as and including 75 mm to 125 mm. In some instances, the bar magnets have a length of 76.2 mm. The width of the bar magnets may also vary, and can range in some cases from 5 mm to 20 mm, such as from 6 mm to 18 mm, and including from 10 to 15 mm. In some cases, the magnets have a width of 12.7 mm. The thickness of the magnets may also vary, and can range in some cases from 0.5 mm to 10 mm, such as from 1 mm to 5 mm, and including 2 mm to 4 mm. In some instances, the width of the magnets is 3.175 mm. In other embodiments, the magnets are ring magnets. In some such embodiments, the ring magnets are arranged concentrically, e.g., in a bullseye configuration. Magnets of the plurality of magnets may be the same shape or different shapes. In some embodiments, the magnets have different shapes and/or sizes.

The positioning of the magnets relative to each other within the retrieval magnet assembly may vary. In some cases, the distance between adjacent magnets within the plurality of magnets ranges from 0.1 mm to 20 mm, such as 1 mm to 15 mm, and including 1 to 9 mm. In some cases, adjacent magnets may be separated by 1 mm, 2 mm, 3 mm, 4 mm, 5 mm, 6 mm, 7 mm, 8 mm, 9 mm, 10 mm, 11 mm or 12 mm, or any intermediate non-integer value between any two of these values. The distance separating adjacent magnets may be the same or different. In some cases, each magnet in the retrieval magnet assembly is separated from an adjacent magnet by the same distance. In other cases, at least one magnet is separated from an adjacent magnet by a distance that is different relative to the distance separating another pair of adjacent magnets. In one example involving four bar magnets, the first and second magnets are separated by 2 mm, the second and third magnets are separated by 8 mm, and the third and fourth magnets are separated by 2 mm.

In embodiments, magnets of the plurality have alternating polarities. Without being bound by theory, it is believed that alternating the polarity of the magnets allows magnetic field lines coming from the North pole of the adjacent magnet pass through the cartridge prior to entering the South pole. For example, in some embodiments, adjacent magnets in the retrieval magnet assembly are arranged in a N, S, N, S . . . configuration, where “N” refers to North and “S” refers to South, and the pattern may repeat for as many magnets are in the plurality. In alternative embodiments, the magnets in the retrieval magnet assembly are arranged in a S, N, S, N . . . configuration.

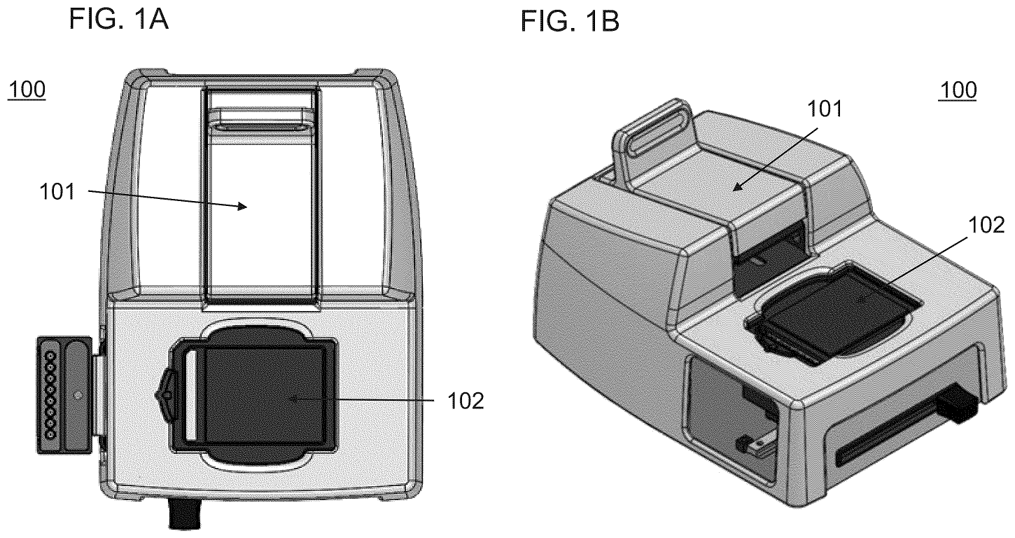

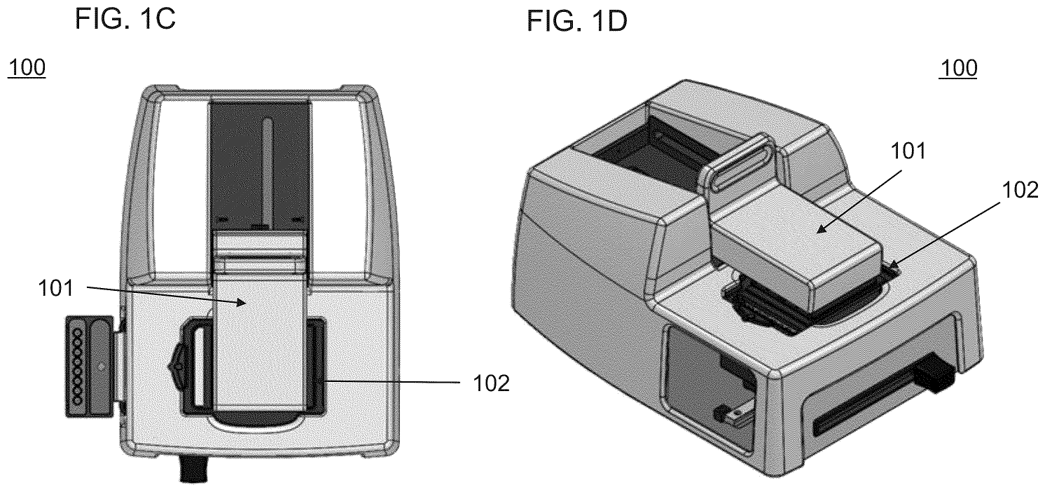

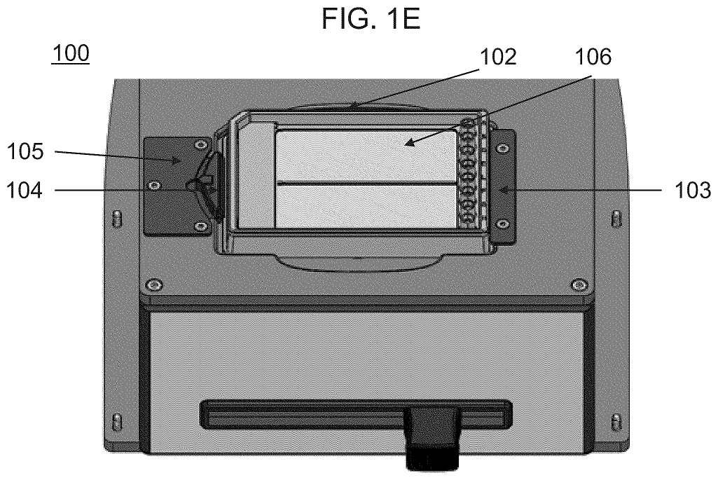

FIG. 1A-1E depict the retrieval magnet assembly and the tray according to certain embodiments of the invention. FIG. 1A and FIG. 1B show different views of system 100 having retrieval magnet assembly 101 in the inactive position. The retrieval magnet assembly 101 is positioned at a distance from tray 102. FIG. 1C and FIG. 1D show different views of system 100 having retrieval magnet assembly 101 in the active position in which the retrieval magnet assembly 101 is positioned adjacent to the tray 102. FIG. 1E presents another view of system 100 in which latch 104, multi-microwell-array-flowcell cartridge 106, latch plate 105, and cartridge catch 103 are shown. Latch 104 and cartridge catch 103 are configured to maintain multi-microwell-array-flowcell cartridge 106 within tray 102. Latch 104 is spring-loaded and can be pushed back for the removal of multi-microwell-array-flowcell cartridge 106.

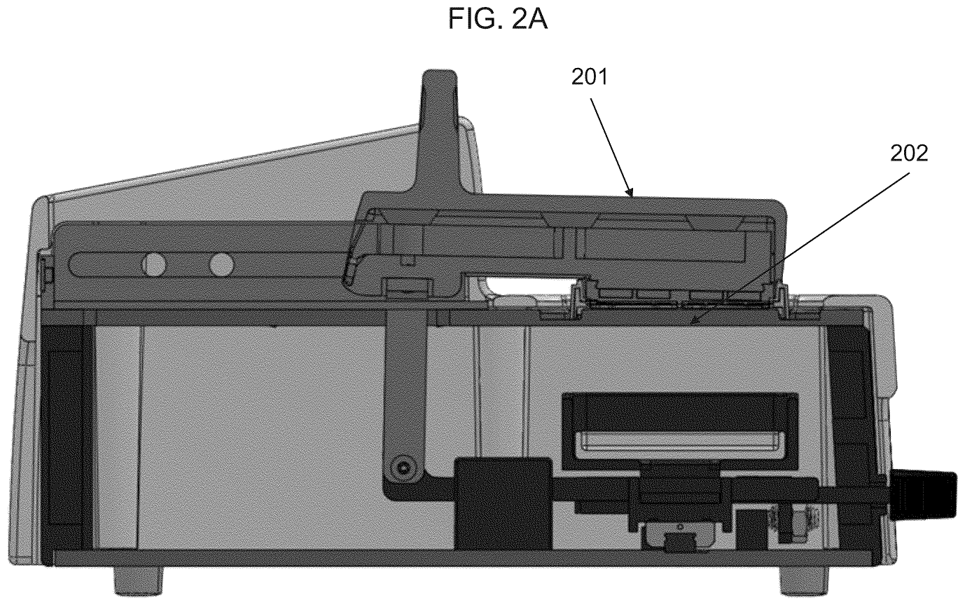

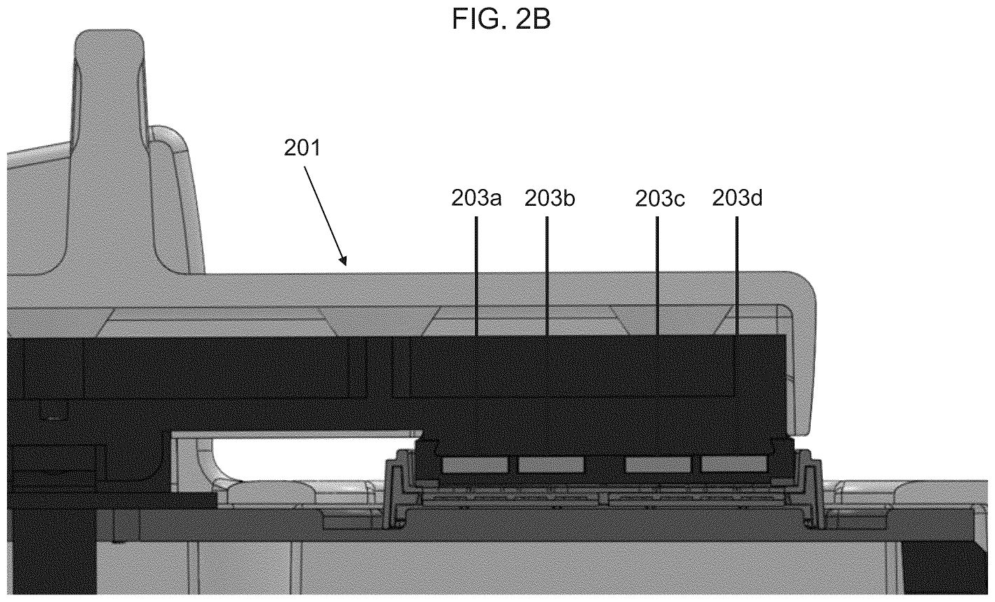

FIG. 2A-2B present different views of the retrieval magnet assembly according to certain embodiments. FIG. 2A depicts retrieval magnet assembly 201 in the active position. Retrieval magnet assembly 201 is positioned in a superior position with respect to tray 202. FIG. 2B depicts the plurality of magnets within retrieval magnet assembly 201. Magnets 203a-203d are bar magnets that have alternating polarities and are arranged in a N, S, N, S configuration.

In some embodiments, systems also include a sample collection vessel holder configured to receive a plurality of sample collection vessels for collecting an analyte from the cartridge. The sample collection vessel holder is configured to be received within the subject systems at a location that is inferior to the tray—and the cartridge, when present—and position sample collection vessels such that liquid from the cartridge may be received in the sample collection vessels. In select embodiments, the sample collection vessel holder comprises a counterweight configured to maintain the sample collection vessel holder in an upright position. In other words, the sample collection vessel holder is not easily tipped over when the sample collection vessel holder is removed from the system. The counterweight may be located, in some instances, in a position that is inferior to the location in which the sample collection vessels are received. In some instances, the system (e.g., a drawer of the system) and the sample collection vessel holder have complementary shapes such that the sample collection vessel holder may be received in the system in a single orientation. In other words, the sample collection vessel holder has a poka-yoke design. Additional details regarding the sample collection vessel holder of the invention are provided below.

In some embodiments, systems additionally include a plurality of sample collection vessels within the sample collection vessel holder. The sample collection vessels may be any suitable liquid container. Suitable sample collection vessels can include, but are not limited to, test tubes, conical tubes, multi-compartment vessels such as microtiter plates (e.g., 96-well plates), centrifuge tubes, culture tubes, microtubes, caps, cuvettes, bottles, rectilinear polymeric vessels, and bags, among other types of vessels. In some cases, the sample collection vessels are test tubes. The opening to each collection vessel may, in certain instances, include a cover, such as a valve, which can be reversibly closed as desired. The sample collection vessel holder may contain any suitable number of sample collection vessels, such as where the number of sample collection vessels ranges from 2 to 10, including 7 to 9. In some cases, the system comprises 8 sample collection vessels.

In some cases, systems also include a waste collection vessel for collecting liquid waste from the multi-microwell-array-flowcell cartridge. The waste collection vessels can include, but are not limited to, conical tubes, centrifuge tubes, culture tubes, bottles, rectilinear polymeric vessels, and bags, among other types of vessels.

In some embodiments, systems of the invention include a drawer configured to hold one or more items, the drawer being movable between a plurality of different positions. In certain instances, the drawer is configured to receive at least one of the sample collection vessel holder and the waste collection vessel. In some such instances, the drawer is configured to receive both the sample collection vessel holder and the waste collection vessel. In some embodiments, the drawer is movable to at least one position in which at least one (including all) of the sample collection vessels within the sample collection vessel holder and/or the waste collection vessel is aligned with one or more outlets of the cartridge. In some embodiments, the system includes a drawer actuator, the drawer actuator being movable between a plurality of different positions, wherein at least some movements of the drawer actuator cause movement of the drawer. In some embodiments, the drawer can be movable along a guiderail. In some embodiments, the guiderail is positioned to engage a top surface of the drawer, a side surface of the drawer, or a bottom surface of the drawer. In some embodiments, the guiderail is positioned to reduce contact from spilling or splashing liquids.

Systems may also include a drip tray designed to cover the linear rail and bear liquid while the cartridge is not over the waste or sample collection vessels. In certain cases, the drip tray is detachable and removable (e.g., for cleaning). In select cases, the drip dray has high bordering walls and an ergonomic handle for removal to ensure liquid stays confined to the tray. In certain instances, the drip tray is moveable along the guiderail and is actuatable by the drawer actuator.

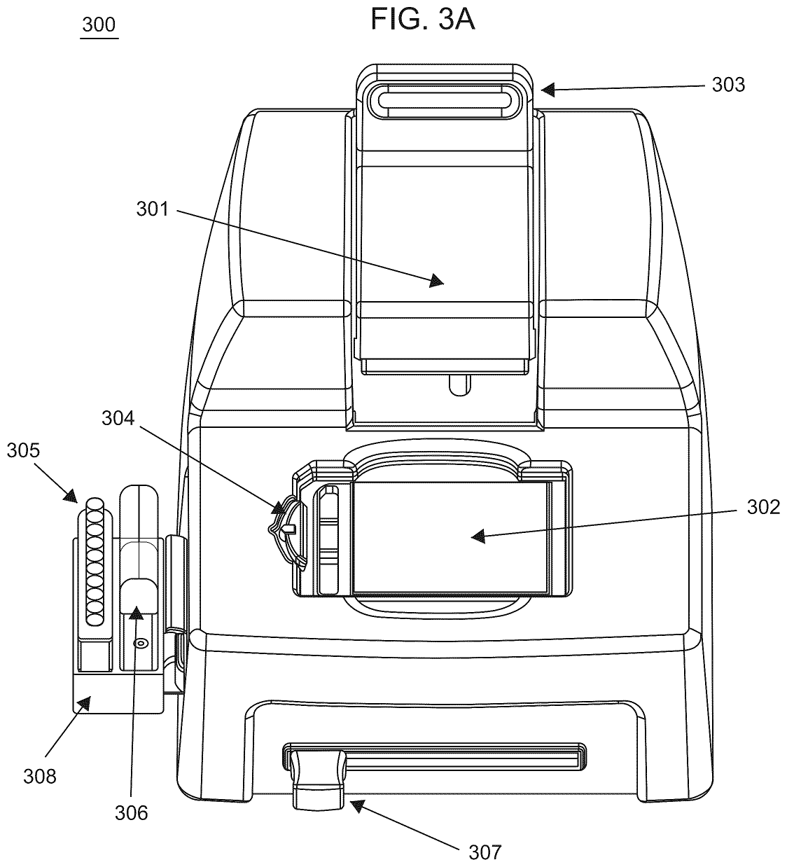

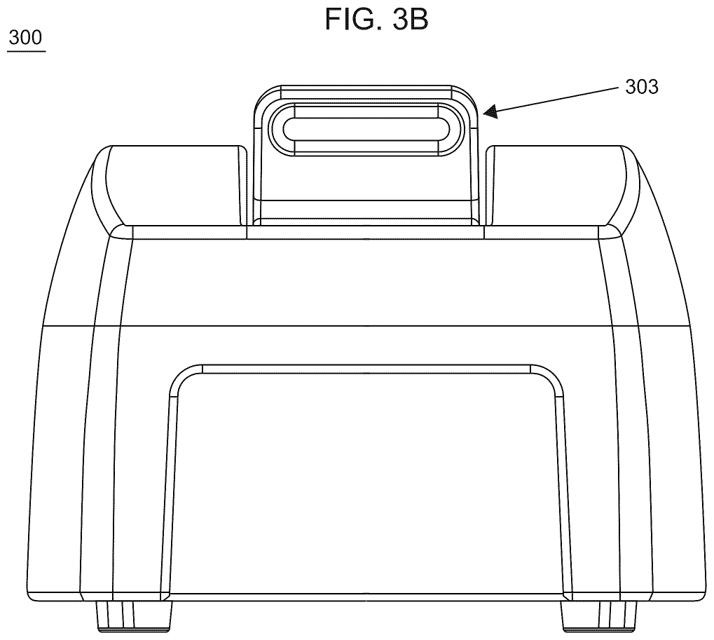

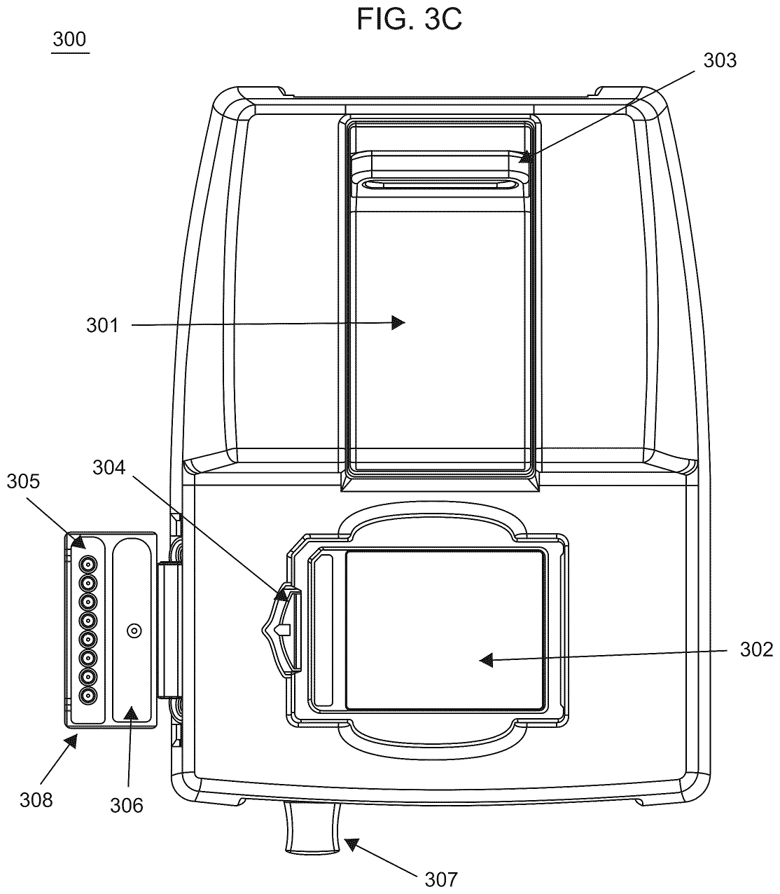

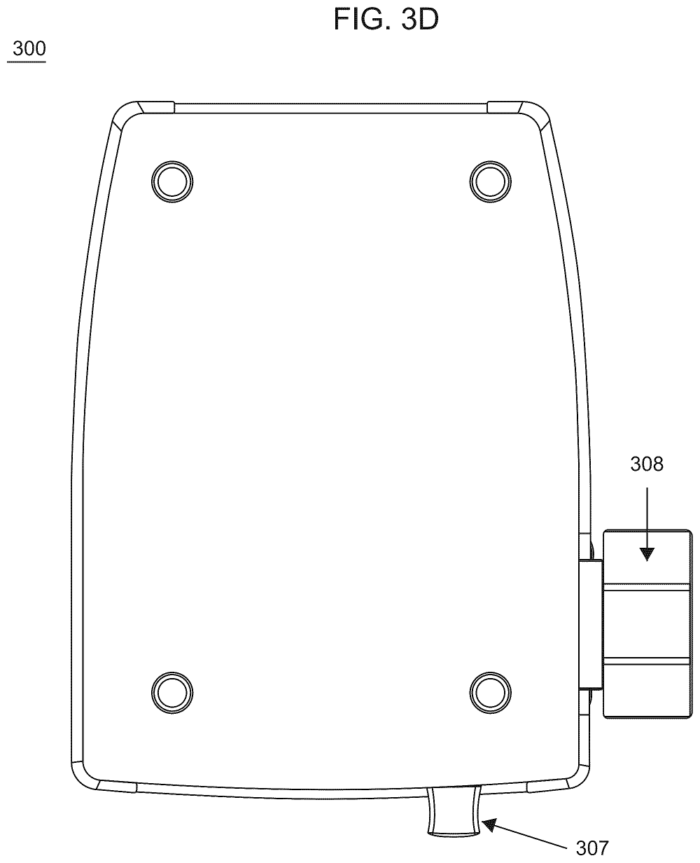

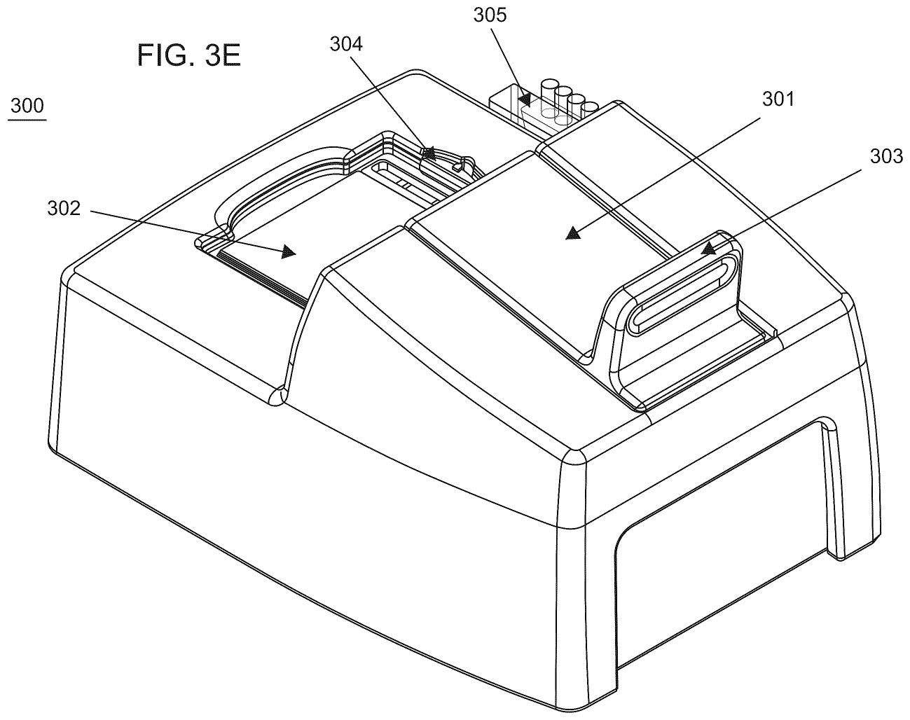

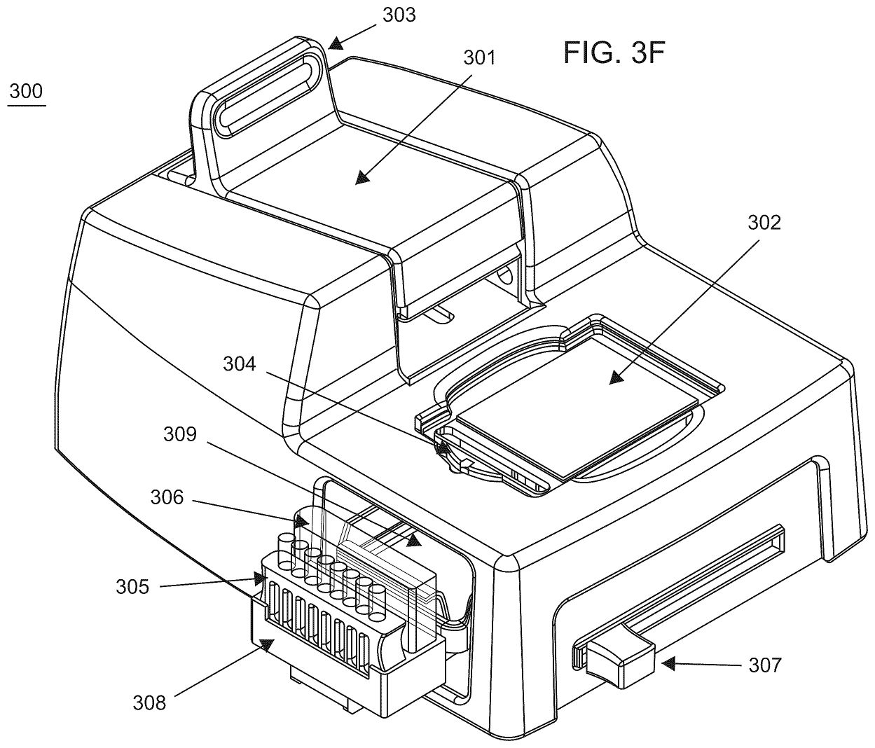

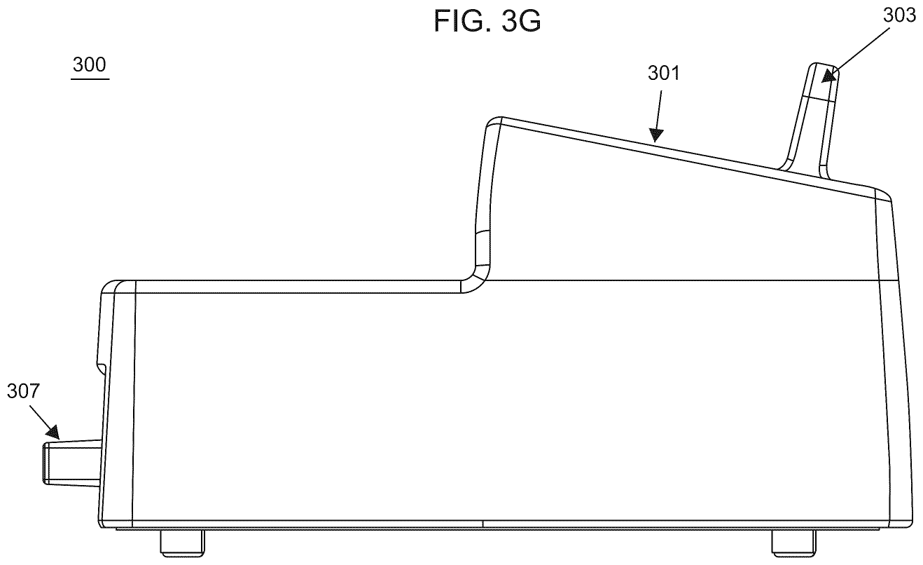

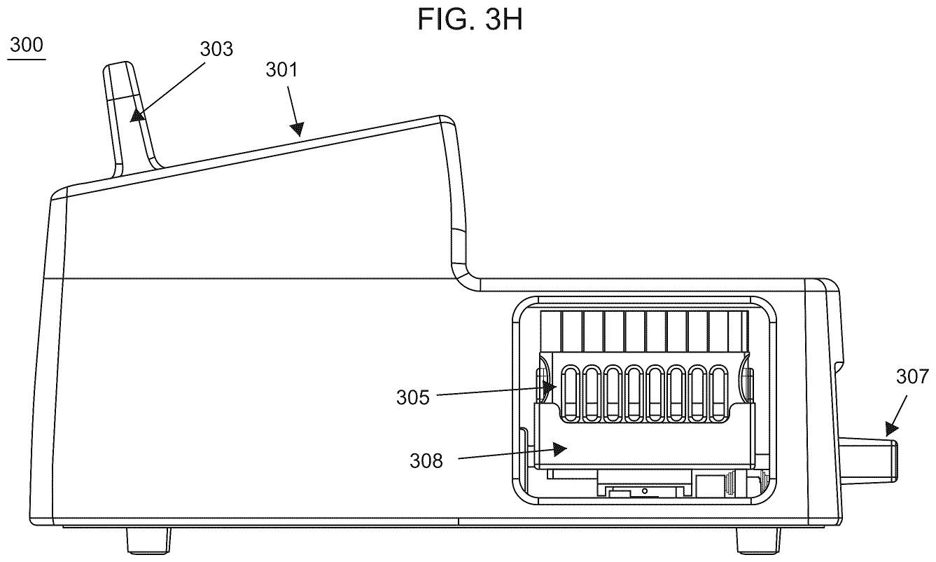

FIG. 3A-3H present different views of a system for processing a cellular sample according to certain embodiments of the invention. FIG. 3A shows a view from the front of system 300, FIG. 3B shows a view from the back of system 300, FIG. 3C shows a view from the top of system 300, FIG. 3D shows a view from the bottom of system 300, FIG. 3E-3F show 45° angle shots of system 300, and FIG. 3G-3H show side profile views of system 300. Included in system 300 are retrieval magnet assembly 301, tray 302, retrieval magnet assembly actuator 303, latch 304, sample collection vessel holder 305, waste collection vessel 306, drawer actuator 307, drawer 308, and drip tray 309. Retrieval magnet assembly 301 is actuatable to an active position via retrieval magnet assembly actuator 303. When in the active position, retrieval magnet assembly 301 is configured to apply a uniform magnetic force to a multi-microwell-array-flowcell cartridge when such is located within tray 302. System 300 additionally includes latch 304 configured to maintain the multi-microwell-array-flowcell cartridge within the tray 302. In this embodiment, latch 304 is a spring-loaded latch that is configured to be pushed backwards when the multi-microwell-array-flowcell cartridge is loaded into the tray 302 and exert a force onto the cartridge and thereby maintain its position.

In the examples of FIG. 3A-3H, drawer 308 includes sample collection vessel holder 305 as well as waste collection vessel 306. Drawer 308 has recesses that are complementary to the shapes of sample collection vessel holder 305 and waste collection vessel 306 such that sample collection vessel holder 305 and waste collection vessel 306 can only fit within drawer 308 in a single orientation. Drawer 308 is operably connected to drawer actuator 307, which is configured to move drawer 308 between different positions within system 300. For example, drawer 308 can be moved to a first position in which the drawer is located outside system 300 (shown in FIG. 3A-3H). In the first position, the waste collection vessel, sample collection vessel(s) and/or the sample collection vessel holder may be added, removed, or exchanged, as necessary. Using drawer actuator 307, drawer 308 may be moved to a sample collection position in which liquid may be received in one or more sample collection vessels within sample collection vessel holder 305, or a waste retrieval position in which liquid may be received in waste collection vessel 306. Drawer 308 may also be moved via drawer actuator 307 to one or more neutral positions in which the drawer is located within system 300 but is not positioned to receive liquid. FIG. 3F depicts drip tray 309 configured to collect liquid that is not received in either the sample collection vessel(s) or the waste collection vessel.

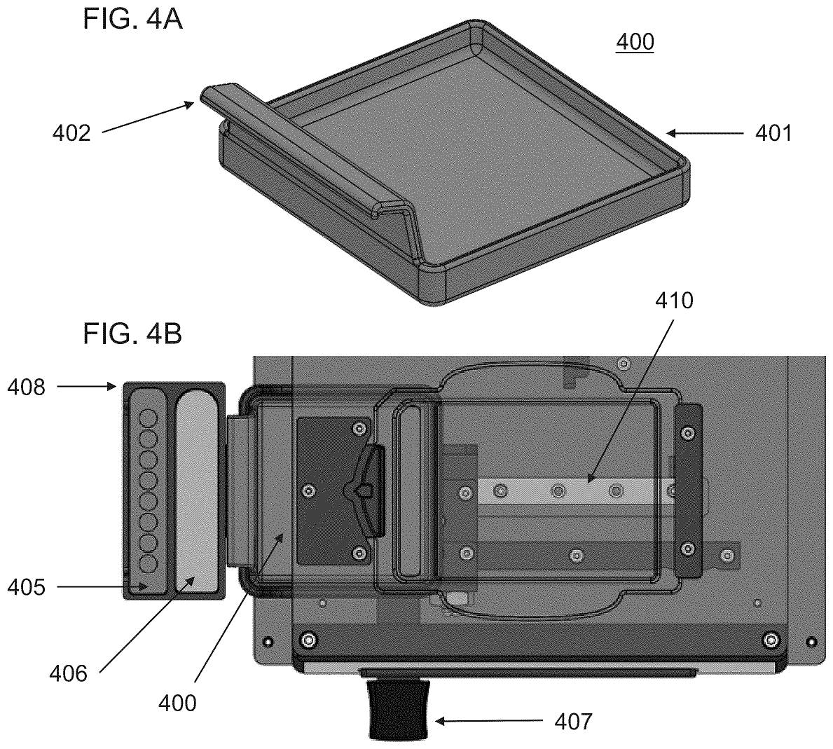

FIG. 4A-4B depict the trip tray, as well as the manner in which the drip tray and drawer are moved through the systems of the invention. FIG. 4A shows drip tray 400, which includes an ergonomic handle 402 to facilitate removal and/or handling of drip tray 400, as well as high bordering walls 401 to ensure liquid stays confined to the tray. FIG. 4B depicts the location of drip tray 400 relative to other components of the subject systems, according to certain embodiments. As shown in FIG. 4B, tray 400 is operably connected to drawer actuator 407 such that movement of drawer actuator 407 causes both drawer 408—along with sample collection vessel holder 405 and waste collection vessel 406, when present—and tray 400 to move through the system along guiderail 410.

Aspects of the subject systems additionally include an interlock configured to prevent the collection of sample liquid into the waste collection vessel when the retrieval magnet assembly is in the active position. As discussed above, the retrieval magnet assembly may be actuated to the active position (e.g., by a user) when it is desirable to obtain sample liquid from the cartridge (e.g., in one or more sample collection vessels). The inventors have discovered that a common error among users of conventional systems is that the sample is sometimes retrieved into a waste container as opposed to sample collection tubes. The interlock described herein is configured to prevent a mis-retrieval from happening by locking the drawer in place during the retrieval operation. In other words, the drawer cannot be moved when the retrieval magnet is in the active position because the interlock restricts its movement. In some embodiments, the drawer is restricted in a position in which the sample collection vessels in the sample collection vessel holder, when present, are positioned under the outlets of the cartridge such that sample liquid is received in the sample collection vessels. In some cases, the retrieval magnet assembly is operably connected to the interlock such that movement (i.e., actuation) of the retrieval magnet assembly also causes movement of the interlock. The interlock engages with the drawer and prevents its movement (e.g., along the guiderail). Movement of the retrieval magnet assembly back to the inactive position causes the interlock to disengage with the drawer such that the drawer may move freely when actuated by the drawer actuator.

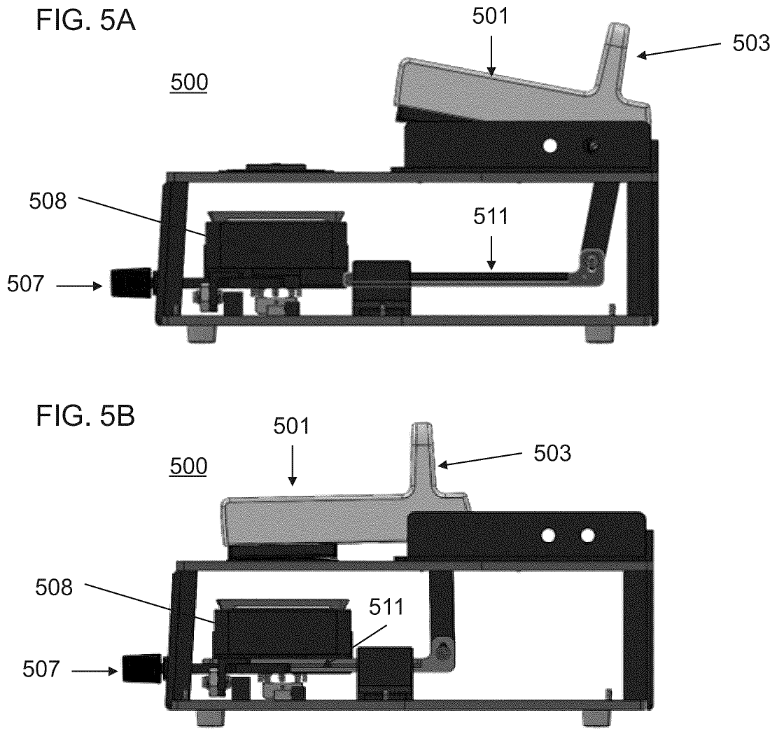

FIG. 5A-5B depict an interlock according to certain embodiments. System 500 includes retrieval magnet assembly 501, retrieval magnet assembly actuator 503, drawer actuator 507, drawer 508 and interlock 511 operably connected to retrieval magnet assembly 501. FIG. 5A shows retrieval magnet assembly 501 in the inactive position.

Interlock 511 is not engaged with drawer 508, and said drawer is free to be moved via drawer actuator 507. FIG. 5B shows retrieval magnet assembly 501 in the active position. Interlock 511 is engaged with drawer 508, and the movement of drawer 508 is consequently restricted.

### Multi-Microwell-Array-Flowcell Cartridges

As described above, aspects of the invention additionally include multi-microwell-array-flowcell cartridges. The multi-microwell-array-flowcell cartridge (also referred to herein as the “cartridge”) of interest includes a plurality of fluidic lanes, each fluidic lane comprising an inlet for receiving liquid, a flow cell comprising a microwell array, and an outlet for discharging liquid. By “multi-microwell-array-flowcell” it is meant that the cartridge possesses a plurality of (i.e., multiple) flowcells, and that each flowcell comprises a microwell array. Each flow cell is a component of a fluidic lane, i.e., a path through which fluid may pass from an inlet to an outlet. The microwell arrays of the subject cartridges, when used for cell capturing, may result in a cell capture rate of 50% or more, such as 55% or more, such as 60% or more, such as 65% or more, such as 70% or more, such as 75% or more, such as 80% or more, such as 85% or more, such as 90% or more, and including 95% or more. In some cases, the cartridges of the invention can be re-used. For example, if one lane of the cartridge is used for a particular assay, the remaining lanes of the cartridge may be employed for the same or a different assay at one or more different time points. In some cases, a single multi-microwell-array-flowcell cartridge may be employed 1 or more times, such as 2 or more times, such as 3 or more times, such are 4 or more times, such as 5 or more times, such as 6 or more times, such as 7 or more times, and including 8 or more times. In select cases, a partially used cartridge (i.e., a cartridge in which one or more lanes have been used for an assay) is stable (i.e., remains in operable condition) for 1 month or more, such as 2 months or more, such as 3 months or more, such as 4 months or more, such as 5 months or more, and including 6 months or more. In some embodiments, use of the multi-microwell-array-flowcell cartridge results in a minimal batch effect between fluidic lanes, including no batch effect.

The number of fluidic lanes of the cartridge may vary. In certain cases, the multi-microwell-array-flowcell cartridge comprises a number of fluidic lanes ranging from 2 to 20, such as 2 to 15 and including 2 to 10. For example, in embodiments, the cartridge comprises 2 lanes, 3 lanes, 4 lanes, 5 lanes, 6 lanes, 7 lanes, 8 lanes, 9 lanes or 10 lanes. In certain embodiments, the multi-microwell-array-flowcell cartridge comprises 8 fluidic lanes. Each fluidic lane includes a flowcell, which includes a microwell array. Further details regarding microwell arrays and flowcells are provided below. In some cases, cartridges include multiple subsets of flowcells. In other words, the plurality of fluidic lanes may be comprised of smaller sets of fluidic lanes which may be manufactured separately. Each subset may include a number of flowcells ranging from 2 to 6, such as 3 to 5. In some cases, each subset includes 4 flowcells.

The inlet of each fluidic lane may vary. In some instances, the inlet is comprised of a gasket. In some instances, the inlet includes a gasket that is configured to taper lock on the sides of a pipette tip (e.g., when the pipette tip is introduced into the inlet so that liquid may be introduced to the fluidic channels). The gasket may be configured such that said taper lock has an overall Z-axis tolerance ranging from 0.1 mm to 1 mm, such as 0.2 mm to 0.8 mm, such as 0.3 mm to 0.7 mm, and including 0.4 mm to 0.6 mm. In some instances, the taper lock has an overall Z-axis tolerance of 0.5 mm. In select cases, the Z-axis tolerance is sufficient to account for pipette tip height variation across different pipette tips. The hardness of the gasket may vary. In some cases, the gasket has a Shore durometer ranging from 1 to 100, such as 5 to 90, and including 10 to 80. The inlet may be constructed from any suitable material, including but not limited to silicon, fused-silica, glass, any of a variety of polymers, e.g. polydimethylsiloxane (PDMS; elastomer), polymethylmethacrylate (PMMA), polycarbonate (PC), polypropylene (PP), polyethylene (PE), high density polyethylene (HDPE), polyimide, cyclic olefin polymers (COP), cyclic olefin copolymers (COC), polyethylene terephthalate (PET), epoxy resins, non-stick materials such as teflon (PTFE), metals (e.g. aluminum, stainless steel, copper, nickel, chromium, and titanium), or any combination thereof.

The outlet of each fluidic lane may similarly vary. In some instances, the outlet is configured in such a manner to induce droplet formation. In some such instances, the outlet is configured in such a manner to induce droplet formation over a fluid flow rate ranging from 5 μL/s to 1,000 μL/s, such as 10 L/s to 700 μL/s, such as 15 μL/s to 600 μL/s, and including 20 μL/s to 500 μL/s. In select embodiments, the outlet is a tapered conical orifice. Said orifice is designed to accommodate a wide variety of fluids with differing surface energies and maintain a uniform droplet of fluid, by having a particular inner diameter, outer diameter, taper angle, and length. In some instances, the outlet has an inner diameter ranging from 0.500 mm to 1.200 mm, such as 0.550 mm to 0.650 mm, and including 0.750 mm to 1.050 mm. In some cases, the outlet has an outer diameter ranging from 1.000 mm to 2.500 mm, such as 1.200 mm to 1.300 mm, and including 1.400 mm to 1.600 mm. In some versions, the outlet has a taper angle ranging from 2° to 20°, such as 3° to 5°, such as 9° to 11°, and including 4° to 6°. In some cases, the outlet has a length ranging from 2.500 mm to 5.500 mm, such as 2.750 mm to 3.250 mm, and including 3.900 mm to 4.100 mm. The outlet may be constructed from any suitable material, including but not limited to silicon, fused-silica, glass, any of a variety of polymers, e.g. polydimethylsiloxane (PDMS; elastomer), polymethylmethacrylate (PMMA), polycarbonate (PC), polypropylene (PP), polyethylene (PE), high density polyethylene (HDPE), polyimide, cyclic olefin polymers (COP), cyclic olefin copolymers (COC), polyethylene terephthalate (PET), epoxy resins, non-stick materials such as teflon (PTFE), metals (e.g. aluminum, stainless steel, copper, nickel, chromium, and titanium), or any combination thereof.

In some cases, the outlet is a stepped outlet. By “stepped” it is meant that the outlet comprises a raised portion that fluid must travel over prior to being discharged. In select embodiments, the stepping of the outlet is sufficient to prevent siphoning of liquid from the flow cell. In some instances, outlets are constructed with a siphon lid. In such instances, the siphon lid is configured to create a channel along the raised portion of the stepped outlet, such that fluid is directed to the outlet and discharged.

The inlet and outlet features of the cartridge can be designed to provide convenient and leak-proof fluid connections with the instrument, or can serve as open reservoirs for manual pipetting of samples and reagents into or out of the cartridge. Examples of convenient mechanical designs for the inlet and outlet port connectors can include, but are not limited to, threaded connectors, Luer lock connectors, Luer slip or “slip tip” connectors, press fit connectors, and the like. The inlets and outlets of the cartridge can further comprise caps, spring-loaded covers or closures, or polymer membranes that can be opened or punctured when the cartridge is positioned in the instrument, and which serve to prevent contamination of internal cartridge surfaces during storage or which prevent fluids from spilling when the cartridge is removed from the instrument. The outlets of the cartridge can further comprise a removable sample collection chamber that is suitable for interfacing with stand-alone PCR thermal cyclers or sequencing instruments.

In some embodiments, the inlets and the outlets can be capable of directing a flow of a fluid through the fluidic channel, thereby contacting the microwells with the fluid.

In some embodiments, the device comprises a pipette tip interface for loading or removing a cell sample, an assay reagent, a bead suspension, waste from the device, or a combination thereof. The device can comprise the cell sample, the assay reagent, the bead suspension, or a combination thereof.

Cartridges of the invention may additionally include a housing. Housings of interest are configured to contain the components of the cartridge such as the flowcells, inlets and outlets. The housing may be comprised of any convenient material, including but not limited to silicon, fused-silica, glass, any of a variety of polymers, e.g. polydimethylsiloxane (PDMS; elastomer), polymethylmethacrylate (PMMA), polycarbonate (PC), polypropylene (PP), polyethylene (PE), high density polyethylene (HDPE), polyimide, cyclic olefin polymers (COP), cyclic olefin copolymers (COC), polyethylene terephthalate (PET), epoxy resins, non-stick materials such as teflon (PTFE), metals (e.g. aluminum, stainless steel, copper, nickel, chromium, and titanium), or any combination thereof. In some cases, the outlets are comprised within the housing.

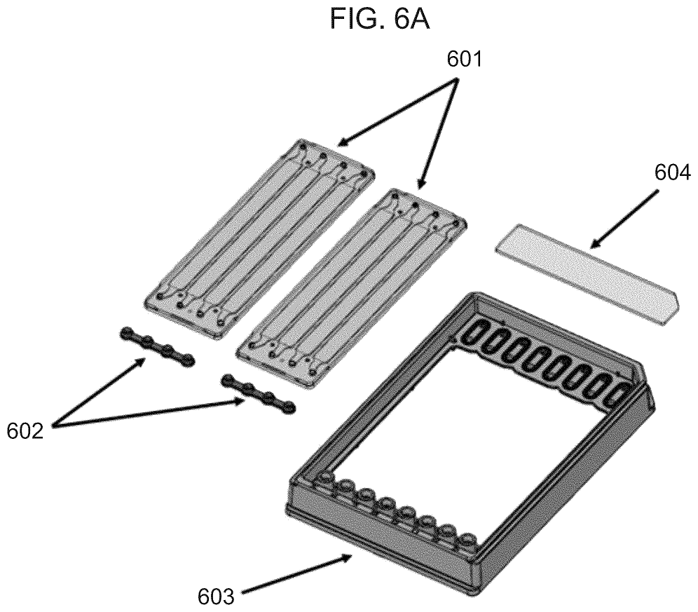

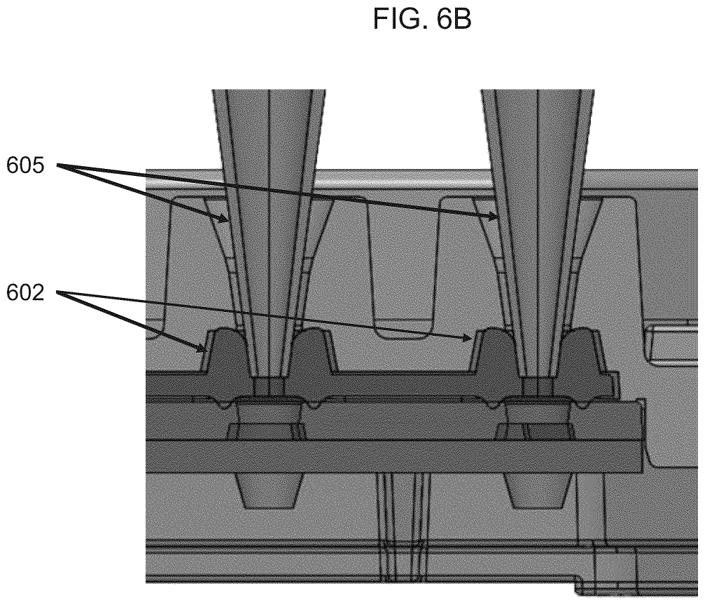

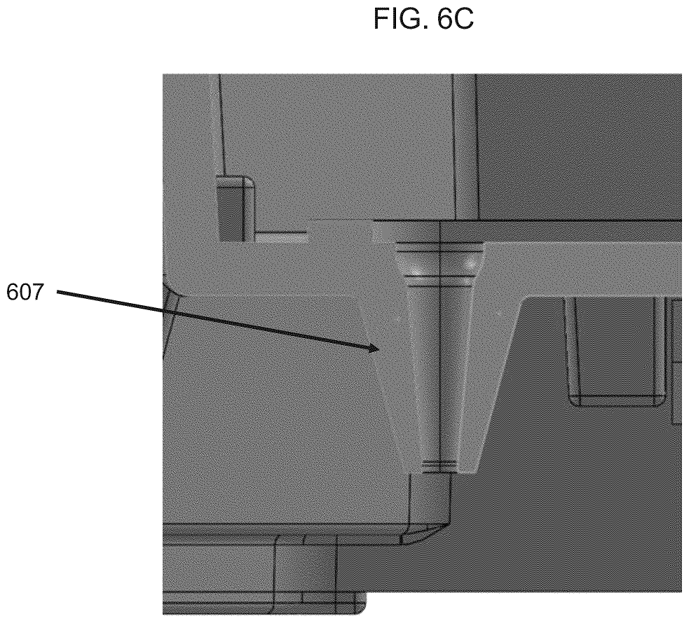

FIG. 6A-6C depict an embodiment of the claimed cartridge. FIG. 6A shows an exploded view of a cartridge including sets of flowcells 601, gaskets 602, and a housing 603. Also shown is siphon lid 604 configured to be placed adjacent to the outlets within housing 603 to create a channel over the raised portion of the outlets. FIG. 6B presents an alternate view of gaskets 602. As shown in FIG. 6B, gaskets 602 are creating a taper lock around pipette tips 605 as liquid is being loaded into the fluidic lanes of the cartridge. FIG. 6C shows outlet 607 having a tapered conical orifice.

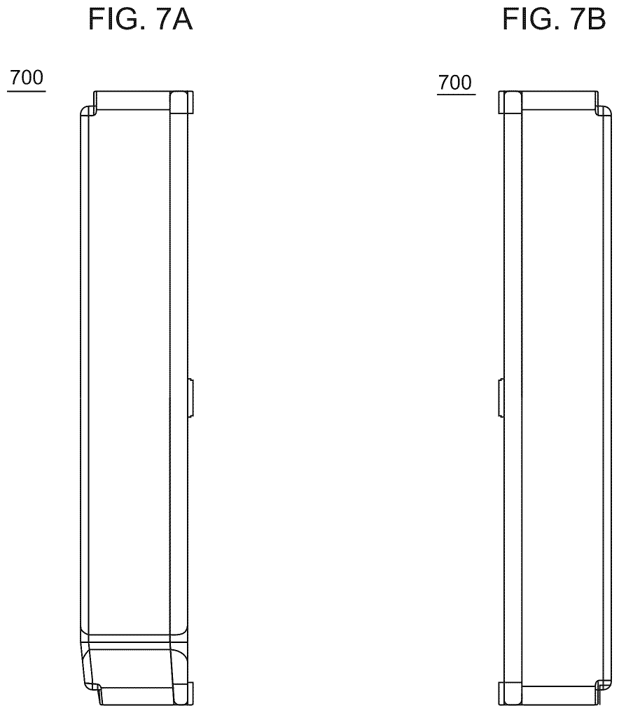

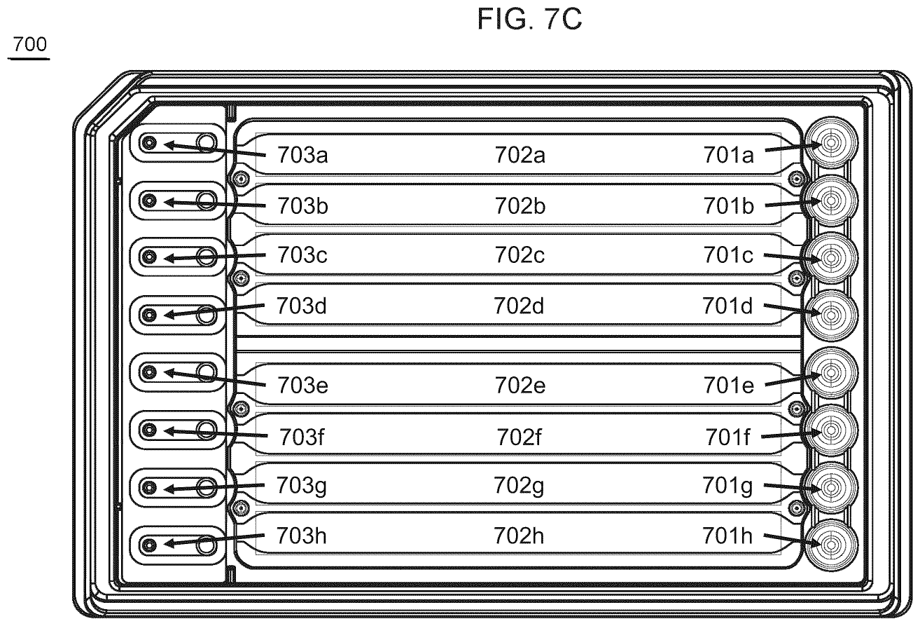

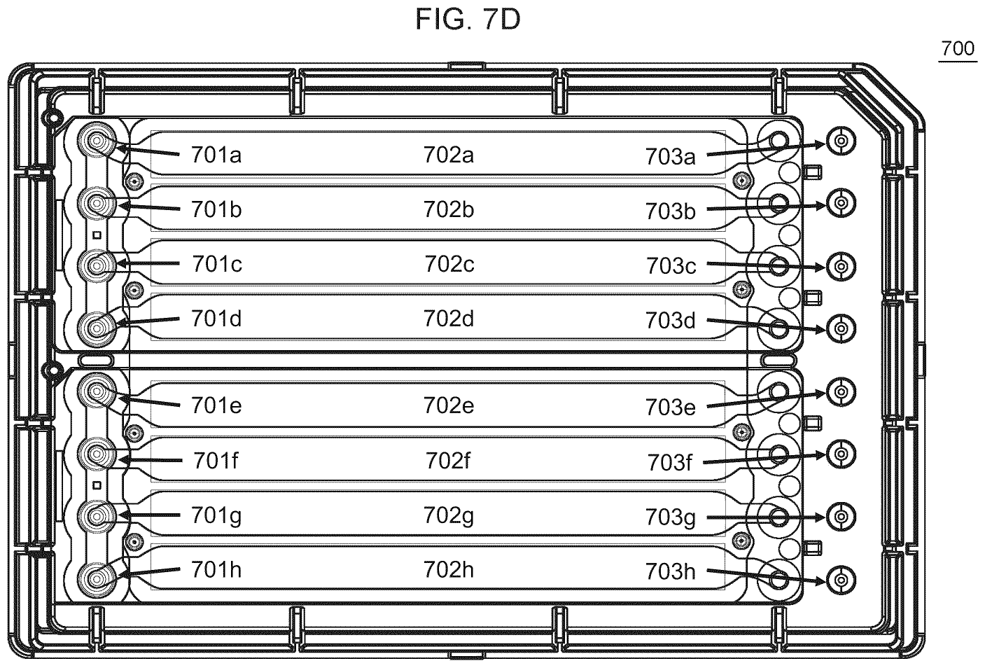

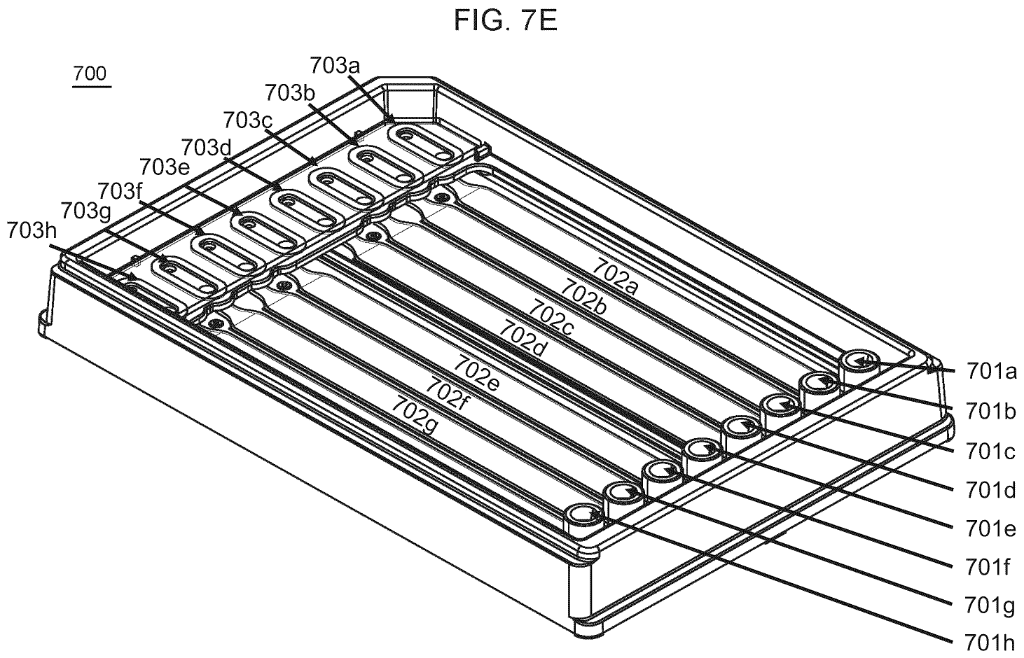

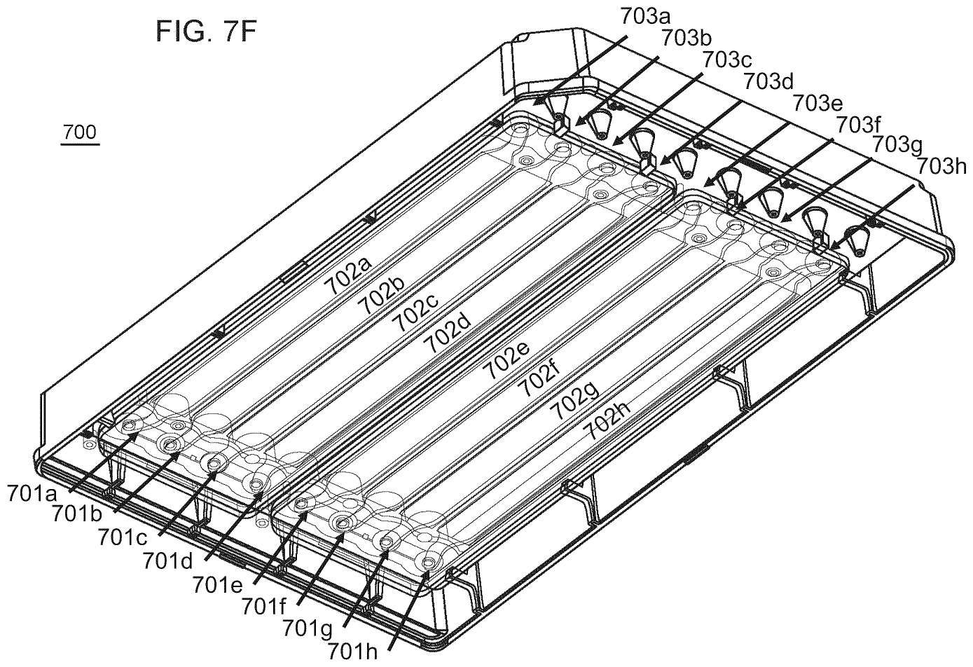

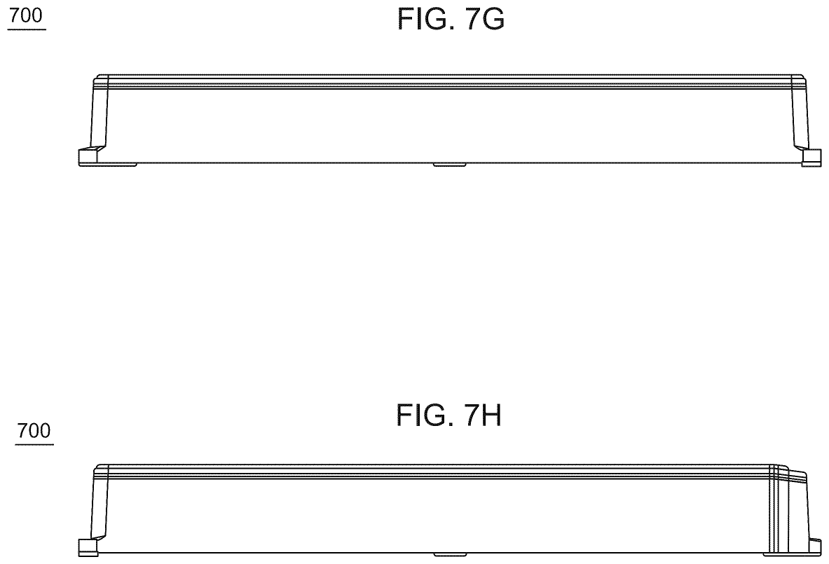

FIG. 7A-7H present different views of a multi-microwell-array-flowcell cartridge according to certain embodiments. FIG. 7A-7B present side profile views of multi-microwell-array-flowcell cartridge 700. FIG. 7C presents a top view of cartridge 700.

Shown in cartridge 700 are inlets 701a-701h, flowcells 702a-702h, as well as outlets 703a-703h. FIG. 7D shows a bottom view of cartridge 700. FIG. 7E presents a 45° angle shot of the top of cartridge 700, while FIG. 7F presents a 45° angle shot of the bottom of cartridge 700. FIG. 7G and FIG. 7H present side profile views of cartridge 700.

In some cases, the cartridge includes one or more alignment features configured to facilitate the assembly of the cartridge, and the positioning of the cartridge in the tray of the system of the invention. For example, in some embodiments, the cartridge of the invention includes a chamfered corner (i.e., a transitional edge between two edges of the cartridge). The chamfered corner may be characterized in some cases by a certain angle relative to the straight edges of the cartridges. In some such cases, the angle may range from 30° to 60°, such as 35° to 55°, such as 40° to 50°, and including 44° to 46°. As discussed above, the tray may be configured such that the cartridge may only be placed in the tray in a single orientation. In other words, the tray and cartridge have corresponding shapes. The cartridge may additionally include aligners such that flowcells may only be attached to the housing in a single orientation. The aligners may, in some cases, take the form of protrusions in the housing of the cartridge. In some embodiments, the tray and cartridge comply with ANSI/SLAS microplate standards. These standards may be accessed at https://www(dot)slas(dot)org/education/ansi-slas-microplate-standards/.

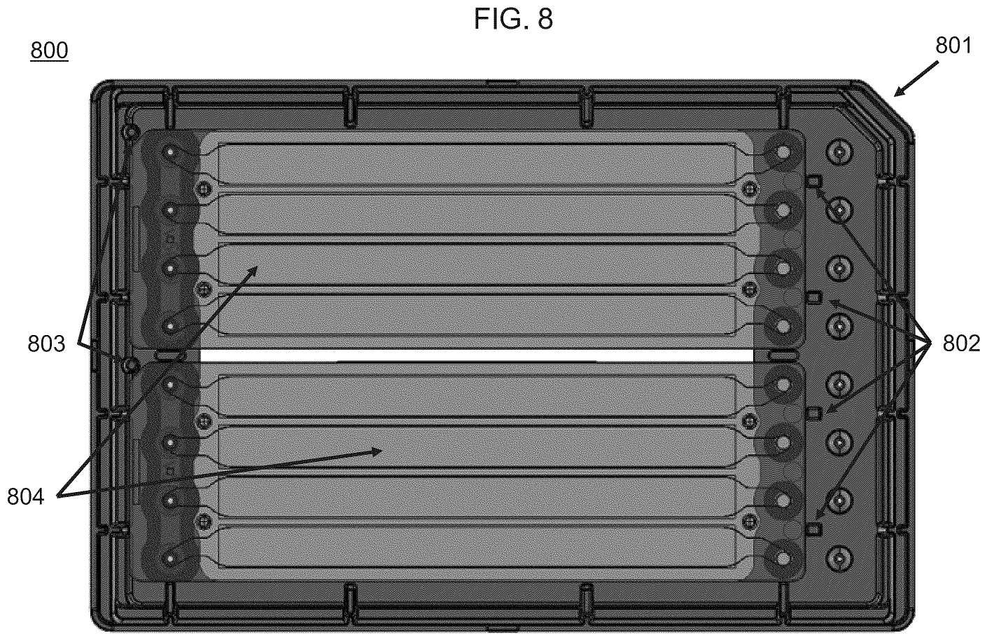

FIG. 8 presents a bottom view of a multi-microwell-array-flowcell cartridge according to certain embodiments of the invention and shows alignment features associated therewith. Cartridge 800 includes a chamfered corner 801 configured for orienting the cartridge 800 in a tray (not shown) of a system of the invention. Cartridge 800 also includes aligners 802 and 803 configured for aligning sets of flowcells 804 when they are installed.

### Flowcells

The design of each flowcell can include a plurality of microarray chambers that interface with a plurality of microwell arrays such that one or more different cell samples can be processed in parallel. The design of the flowcells can further include features for creating consistent (e.g., uniform) flow velocity profiles, i.e. “plug flow”, across the width of the array chamber to provide for more efficient (e.g., uniform) delivery of cells and beads to the microwells, for example, by using a porous barrier located near the chamber inlet and upstream of the microwell array as a “flow diffuser”, or by dividing each array chamber into several subsections that collectively cover the same total array area, but through which the divided inlet fluid stream flows in parallel. In some embodiments, the flowcell can enclose or incorporate more than one microwell array substrate.

In general, the dimensions of fluidic channels and the array chambers in flowcell designs will be optimized to (i) provide efficient (e.g., uniform) delivery of cells and beads to the microwell array, and (ii) to minimize sample and reagent consumption. In embodiments, each flow cell is comprised of an elongate channel. In some cases, the length of the elongate channel may be characterized by a straight line, i.e., the channel does not possess curves. The length of the elongate channels may vary. In certain cases, the range in length from 20 mm to 500 mm, such as 25 mm to 400 mm, such as 30 mm to 300 mm, such as 40 mm to 300 mm, such as 45 mm to 200 mm and including 50 mm to 100 mm. The width of an elongate channel can be different in different implementations, for example, ranging from 0.1 mm to 100 mm. In some embodiments, the width can be, or be about, 0.1, 0.2, 0.3, 0.4, 0.5, 0.6, 0.7, 0.8, 0.9, 1, 2, 3, 4, 5, 6, 7, 8, 9, 10, 20, 30, 40, 50, 60, 70, 80, 90, 100 mm, or a number or a range between any two of these values. In some embodiments, the width can be at least, or at most, 0.1, 0.2, 0.3, 0.4, 0.5, 0.6, 0.7, 0.8, 0.9, 1, 2, 3, 4, 5, 6, 7, 8, 9, 10, 20, 30, 40, 50, 60, 70, 80, 90, or 100 mm.

The height of a fluidic channel can be different in different implementations, for example, ranging from 0.1 mm to 100 mm. In some embodiments, the height can be, or be about, 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 3, 4, 5, 6, 7, 8, 9, 10 mm, or a number or a range between any two of these values. In some embodiments, the height can be at least, or at most, 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 3, 4, 5, 6, 7, 8, 9, or 10 mm.

Flowcells can be fabricated using a variety of techniques and materials known to those of skill in the art. In general, a flowcell can be fabricated as a separate part and subsequently either mechanically clamped or permanently bonded to the microwell array substrate. Examples of suitable fabrication techniques include conventional machining, CNC machining, injection molding, 3D printing, alignment and lamination of one or more layers of laser or die-cut polymer films, or any of a number of microfabrication techniques such as photolithography and wet chemical etching, dry etching, deep reactive ion etching, or laser micromachining.

Once the flowcell part has been fabricated it can be attached to the microwell array substrate mechanically, e.g. by clamping it against the microwell array substrate (with or without the use of a gasket), or it can be bonded directly to the microwell array substrate using any of a variety of techniques (depending on the choice of materials used) known to those of skill in the art, for example, through the use of anodic bonding, thermal bonding, or any of a variety of adhesives or adhesive films, including epoxy-based, acrylic-based, silicone-based, UV curable, polyurethane-based, or cyanoacrylate-based adhesives. In some embodiments, the substrate can form the fluidic channel bottom of the fluidic channel, or the substrate can be on the fluidic channel bottom of the fluidic channel. In some embodiments, the substrate comprises silicon, fused-silica, glass, a polymer, a metal, an elastomer, polydimethylsiloxane, agarose, a hydrogel, or a combination thereof. In some embodiments, the cartridge is an inseparable assembly where the flow cells are irreversibly attached to the housing. In select cases, the flow cell is made by bonding the microwell substrate to the fluidic substrate.

Flowcells can be fabricated using a variety of materials known to those of skill in the art. In general, the choice of material used will depend on the choice of fabrication technique used, and vice versa. Examples of suitable materials include, but are not limited to, silicon, fused-silica, glass, any of a variety of polymers, e.g. polydimethylsiloxane (PDMS; elastomer), polymethylmethacrylate (PMMA), polycarbonate (PC), polypropylene (PP), polyethylene (PE), high density polyethylene (HDPE), polyimide, cyclic olefin polymers (COP), cyclic olefin copolymers (COC), polyethylene terephthalate (PET), epoxy resins, metals (e.g. aluminum, stainless steel, copper, nickel, chromium, and titanium), a non-stick material such as teflon (PTFE), or a combination of these materials. The cyclic olefin polymers (COP) can comprise Zeonor 1020R or Zeonor 1060R.

### Microwell Arrays

In some embodiments, a microwell can comprise a small reaction chamber of defined volume. In some embodiments, a microwell can entrap one or more cells. In some embodiments, a microwell can entrap only one cell. In some embodiments, a microwell can entrap one or more solid supports (e.g., beads). In some embodiments, a microwell can entrap only one solid support. In some embodiments, a microwell entraps a single cell and a single solid support (e.g., bead). In some embodiments, a microwell can contain a single particle (e.g., a cell or a bead). In some embodiments, a microwell can contain two different particles (e.g., a cell and a bead).

Microwells can be fabricated in a variety of shapes. Non-limiting exemplary well geometries can include cylindrical, conical, hemispherical, rectangular, or polyhedral (e.g., three dimensional geometries comprised of several planar faces, for example, hexagonal columns, octagonal columns, inverted triangular pyramids, inverted square pyramids, inverted pentagonal pyramids, inverted hexagonal pyramids, or inverted truncated pyramids). The microwells can comprise a shape that combines two or more of these geometries. For example, a microwell can be partly cylindrical, with the remainder having the shape of an inverted cone. A microwell can include two side-by-side cylinders, one of larger diameter (e.g., that corresponds roughly to the diameter of the beads) than the other (e.g., that corresponds roughly to the diameter of the cells), that are connected by a vertical channel (that is, parallel to the cylinder axes) that extends the full length (depth) of the cylinders. The location of the opening of the microwell can vary. For example, the opening of the microwell can be at the upper surface of the substrate. For example, the opening of the microwell can be at the lower surface of the substrate. The shape of the close end, for example the bottom, of the microwell can vary. For example, the closed end of the microwell can be flat. For example, the closed end of the microwell can have a curved surface (e.g., convex or concave). The shape and/or size of the microwell can be determined based on the types of cells or solid supports to be trapped within the microwells. In some embodiments, a microwell can have a non-circular cross section (e.g., square or hexagonal) in a plane of the substrate.

Microwells can be fabricated in a variety of sizes. Microwell size can be characterized, for example, in terms of the diameter and/or the depth of the microwells. The diameter of the microwell can refer to the largest circle that can be inscribed within the planar cross-section of the microwell geometry. The diameter of the microwells can, in some embodiments, range from about 1-fold to about 10-folds the diameter of the cells or solid supports to be trapped within the microwells. In some embodiments, the microwell diameter can be, or be about, 1-fold, 1.5-fold, 2-folds, 3-folds, 4-folds, 5-folds, 6-folds, 7-folds, 8-folds, 9-folds, 10-folds, or a number or a range between any two of these values, the diameter of the cells or the solid supports to be trapped within the microwells. In some embodiments, the microwell diameter can be at least, or at most, 1-fold, 1.5-fold, 2-folds, 3-folds, 4-folds, 5-folds, 6-folds, 7-folds, 8-folds, 9-folds, 10-folds the diameter of the cells or the solid supports to be trapped within the microwells. In some embodiments, the microwell diameter can be about 2.5-folds the diameter of the cells or solid supports to be trapped within the microwells.

The diameter of a microwell can be specified in terms of absolute dimensions. The diameter of a microwell can range from about 1 nanometer to about 1000 micrometers. In some embodiments, the microwell diameter can be, or be about, 1, 2, 3, 4, 5, 6, 7, 8, 9, 10, 20, 30, 40, 50, 60, 70, 80, 90, 100, 200, 300, 400, 500, 600, 700, 800, 900, 1000 micrometers, or a number or a range between any two of these values. In some embodiments, the microwell diameter can be at least, or at most, 1, 2, 3, 4, 5, 6, 7, 8, 9, 10, 20, 30, 40, 50, 60, 70, 80, 90, 100, 200, 300, 400, 500, 600, 700, 800, 900, 1000 micrometers. In some embodiments, the microwell diameter can be, or be about, 1, 2, 3, 4, 5, 6, 7, 8, 9 10, 20, 30, 40, 50, 60, 70, 80, 90, 100, 200, 300, 400, 500, 600, 700, 800, 900, 1000 micrometers, or a number or a range between any two of these values. In some embodiments, the microwell diameter can be at least, or at most 1, 2, 3, 4, 5, 6, 7, 8, 9, 10, 20, 30, 40, 50, 60, 70, 80, 90, 100, 200, 300, 400, 500, 600, 700, 800, 900, 1,000 micrometers. In some embodiments, the microwell diameter can be about 30 micrometers.

The depth of the microwell can vary, for example, to provide efficient trapping of droplets, for example cells and solid supports, or to provide efficient exchange of assay buffers and other reagents contained within the wells. The ratio of diameter to depth (i.e., aspect ratio) can be varied such that once a cell and/or a solid support settle inside a microwell, they will not be displaced by fluid motion above the microwell. In some embodiments, the depth of the microwell can be smaller than the diameter of the bead. For example, the depth of the microwell can be, or be about, 5%, 10%, 20%, 30%, 40%, 50%, 60%, 70%, 80%, 90%, 99%, 99.9%, 100%, or a number or a range between any two of these values, of the diameter of the bead. For example, the depth of the microwell can be at least, or at most, 5%, 10%, 20%, 30%, 40%, 50%, 60%, 70%, 80%, 90%, 99%, 99.9%, 100% of the diameter of the bead. In some embodiments, synthetic particles such as beads can protrude outside of the microwells.

In some embodiments, a dimension of a microwell allows the microwell to contain at most one bead. A ratio of the width of the microwell to a diameter of the bead can vary, ranging from 1-1.9. In some embodiments, the ratio of the width of the microwell to the diameter of the bead can be, or be about, 1, 1.1, 1.2, 1.3, 1.4, 1.5, 1.6, 1.7, 1.8, 1.9, or a number or a range between any two of these values. In some embodiments, the ratio of the width of the microwell to the diameter of the bead can be at least, or at most, 1, 1.1, 1.2, 1.3, 1.4, 1.5, 1.6, 1.7, 1.8, or 1.9.

The dimensions of a microwell can vary such that the microwell has sufficient space to accommodate a solid support and a cell of various sizes without being dislodged by fluid motion above the microwell. The depth of a microwell can range from about 1-fold to about 10-folds the diameter of the cells or solid supports to be trapped within the microwells. In some embodiments, the microwell depth can be, or be about, 1-fold, 1.5-fold, 2-folds, 3-folds, 4-folds, 5-folds, 6-folds, 7-folds, 8-folds, 9-folds, 10-folds, or a number or a range between any two of these values, the diameter of the cells or solid supports to be trapped within the microwells. In some embodiments, the microwell depth can be about 2.5-folds the diameter of the cells or solid supports to be trapped within the microwells.

An aspect ratio of the width of the microwell to the depth of the microwell can vary, for example ranging from 0.1-2. In some embodiments, the aspect ratio of the width of the microwell to the depth of the microwell can be, or be about, 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, or a number or a range between any two of these values. In some embodiments, the aspect ratio of the width of the microwell to the depth of the microwell can be at least, or at most, 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, or 2.

The depth of a microwell can be specified in terms of its absolute dimension. For example, the depth of a microwell can range from about 1 nanometer to about 1000 micrometers. In some embodiments, the microwell depth can be, or be about, 1, 2, 3, 4, 5, 6, 7, 8, 9, 10, 20, 30, 40, 50, 60, 70, 80, 90, 100, 200, 300, 400, 500, 600, 700, 800, 900, 1,000 micrometers, or a number or a range between any two of these values. In some embodiments, the microwell depth can be at least, or at most, 1, 2, 3, 4, 5, 6, 7, 8, 9, 10, 20, 30, 40, 50, 60, 70, 80, 90, 100, 200, 300, 400, 500, 600, 700, 800, 900, 1,000 micrometers. In some embodiments, the microwell depth can be, or be about, 1, 2, 3, 4, 5, 6, 7, 8, 9 10, 20, 30, 40, 50, 60, 70, 80, 90, 100, 200, 300, 400, 500, 600, 700, 800, 900, 1,000 micrometers, or a number or a range between any two of these values. In some embodiments, the microwell depth can be at least, or at most, 1, 2, 3, 4, 5, 6, 7, 8, 9, 10, 20, 30, 40, 50, 60, 70, 80, 90, 100, 200, 300, 400, 500, 600, 700, 800, 900, 1,000 micrometers. In some embodiments, the microwell depth ranges from 20 micrometers to 60 micrometers, such as 25 micrometers to 55 micrometers, such as 30 micrometers to 50 micrometers, and including 45 micrometers to 49 micrometers. In some instances, the microwell depth is 48 micrometers/The volume of a microwell can vary, for example ranging from about 1 picoliter to about 1,000 microliters. In some embodiments, the microwell volume can be, or be about, 1, 2, 3, 4, 5, 6, 7, 8, 9, 10, 20, 30, 40, 50, 60, 70, 80, 90, 100, 200, 300, 400, 500, 600, 700, 800, 900, 1,000, or a number or a range between any two of these values, picoliters. In some embodiments, the microwell volume can be at least, or at most, 1, 2, 3, 4, 5, 6, 7, 8, 9, 10, 20, 30, 40, 50, 60, 70, 80, 90, 100, 200, 300, 400, 500, 600, 700, 800, 900, or 1,000 picoliters. In some embodiments, the microwell volume can be, or be about, 1, 2, 3, 4, 5, 6, 7, 8, 9, 10, 20, 30, 40, 50, 60, 70, 80, 90, 100, 200, 300, 400, 500, 600, 700, 800, 900, 1000, or a number or a range between any two of these values, nanoliters. In some embodiments, the microwell volume can be at least, or at most, 1, 2, 3, 4, 5, 6, 7, 8, 9, 10, 20, 30, 40, 50, 60, 70, 80, 90, 100, 200, 300, 400, 500, 600, 700, 800, 900, or 1,000 nanoliters. In some embodiments, the microwell volume can be, or be about, 1, 2, 3, 4, 5, 6, 7, 8, 9, 10, 20, 30, 40, 50, 60, 70, 80, 90, 100, 200, 300, 400, 500, 600, 700, 800, 900, 1,000, or a number or a range between any two of these values, microliters. In some embodiments, the microwell volume can be at least, or at most, 1, 2, 3, 4, 5, 6, 7, 8, 9, 10, 20, 30, 40, 50, 60, 70, 80, 90, 100, 200, 300, 400, 500, 600, 700, 800, 900, or 1,000. In some embodiments, the microwell volume can be about 1 microliter.

The volume of a microwell can be characterized in terms of the variation in volume from one microwell to another. The coefficient of variation (expressed as a percentage) for microwell volume can range from about 1% to about 100%. The coefficient of variation for microwell volume can be, or be about, 1%, 2%, 3%, 4%, 5%, 6%, 7%, 8%, 9%, 10%, 20%, 30%, 40%, 50%, 60%, 70%, 80%, 90%, 100%, or a number or a range between any two of these values. The coefficient of variation for microwell volume can be, at least or at most, 1%, 2%, 3%, 4%, 5%, 6%, 7%, 8%, 9%, 10%, 20%, 30%, 40%, 50%, 60%, 70%, 80%, 90%, or 100%. In some embodiments, the coefficient of variation of microwell volume can be about 2.5%.

The ratio of the volume of a microwell to the surface area of a bead (or to the surface area of a solid support to which stochastic barcode oligonucleotides can be attached) can vary, for example range from about 2.5 to about 1,520 micrometers. In some embodiments, the ratio can be, or be about, 2.5, 5, 10, 100, 500, 750, 1000, 1,520 micrometers, or a number or a range between any two of these values. In some embodiments, the ratio can be at least, or at most, 2.5, 5, 10, 100, 500, 750, 1,000, or 1,520 micrometers. In some embodiments, the ratio can be about 67.5 micrometers.

Microwells can be arranged in a one dimensional, two dimensional, or three-dimensional array. A three-dimensional array can be achieved, for example, by stacking a series of two or more two dimensional arrays, for example by stacking two or more substrates comprising microwell arrays.

The pattern and spacing between microwells can vary to optimize the efficiency of trapping a single cell and a single solid support (e.g., bead) in each well, as well as to maximize the number of wells per unit area of the array. The microwells can be distributed according to a variety of random or non-random patterns. For example, they can be distributed entirely randomly across the surface of the array substrate, or they can be arranged in a square grid, rectangular grid, hexagonal grid, or the like.

The center-to-center distance or the center-to-center spacing between wells can vary from about 1 micrometer to about 1,000 micrometers. In some embodiments, the center-to-center distance between wells can be, or be about, 1, 2, 3, 4, 5, 6, 7, 8, 9, 10, 20, 30, 40, 50, 60, 70, 80, 90, 100, 200, 300, 400, 500, 600, 700, 800, 900, 1,000 micrometers, or a number or a range between any two of these values. In some embodiments, the center-to-center distance between wells can be at least, or at most, 1, 2, 3, 4, 5, 6, 7, 8, 9, 10, 20, 30, 40, 50, 60, 70, 80, 90, 100, 200, 300, 400, 500, 600, 700, 800, 900, or 1,000 micrometers. In some embodiments, the center-to-center distance between wells can be about 4,890 micrometers.

The distance or the spacing between the edges of the microwells can vary from about 1 micrometer to about 1,000 micrometers. In some embodiments, the distance between the edges of the wells can be, or be about, 1, 2, 3, 4, 5, 6, 7, 8, 9, 10, 20, 30, 40, 50, 60, 70, 80, 90, 100, 200, 300, 400, 500, 600, 700, 800, 900, 1000 micrometers, or a number or a range between any two of these values. In some embodiments, the distance between the edges of the wells can be at least, or at most, 1, 2, 3, 4, 5, 6, 7, 8, 9, 10, 20, 30, 40, 50, 60, 70, 80, 90, 100, 200, 300, 400, 500, 600, 700, 800, 900, or 1,000 micrometers. In some embodiments, the distance between the edges of the wells can be about 80 micrometers.

A microwell array can comprise microwells at varying densities, for example ranging from 100 microwells per inch² to 1,000,000 microwells per inch². In some embodiments, the density of the microwell array can be, or be about, 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, 100000, 200000, 300000, 400000, 500000, 600000, 700000, 800000, 900000, 1000000, 2000000, 3000000, 4000000, 5000000, 6000000, 7000000, 8000000, 9000000, 10000000, or a number or a range between any two of these values, microwells per inch². In some embodiments, the density of the microwell array can be, or be about, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, 100000, or a number or a range between any two of these values, microwells per cm². In some embodiments, the density of the microwell array can be at least, or at most, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, or 100000 microwells per cm². In some cases, each microwell array comprises a density ranging from 10,000 microwells/cm² to 60,000 microwells/cm², such as 20,000 microwells/cm² to 50,000 microwells/cm², and including 36,000 microwells/cm² to 42,000 microwells/cm².

The total number of microwells on a substrate can vary based on the pattern and the spacing of the wells and the overall dimensions of the array. The number of microwells in the array can vary, for example, ranging from about 96 to about 1000000. In some embodiments, the number of microwells in the microarray can be, or be about, 96, 384, 1536, 2000, 3000, 4000, 5000, 6000, 7000, 8000, 9000, 10000, 20000, 30000, 40000, 50000, 60000, 70000, 80000, 90000, 100000, 200000, 300000, 400000, 500000, 600000, 700000, 800000, 900000, 1000000, 2000000, 3000000, 4000000, 5000000, 6000000, 7000000, 8000000, 9000000, 10000000, 108, 109, or a number or a range between any two of these values. In some embodiments, the number of microwells in the microarray can be at least, or at most, 96, 384, 1536, 2000, 3000, 4000, 5000, 6000, 7000, 8000, 9000, 10000, 20000, 30000, 40000, 50000, 60000, 70000, 80000, 90000, 100000, 200000, 300000, 400000, 500000, 600000, 700000, 800000, 900000, 1000000, 2000000, 3000000, 4000000, 5000000, 6000000, 7000000, 8000000, 9000000, 10000000, 108, 109. In some embodiments, the number of microwells in the microwell array can be about 96. In some embodiments, the number of microwells can be about 150000. In some instances, each microwell array comprises from 100,000 microwells to 500,000 microwells, such as 200,000 microwells to 250,000 microwells, and including 250,000 microwells to 300,000 microwells. In some instances, each microwell array comprises at least 220,000 microwells per lane.

Microwells of the microwell array may additionally have any suitable pitch, where pitch describes the amount of separation between microwells. For example, in some cases, a calculation of pitch includes well cross-section and wall separation. In some cases, pitch ranges from 1 μm to 500 μm, such as 5 μm to 450 μm, such as 10 μm to 400 μm, such as 15 μm to 350 μm, such as 20 μm to 300 μm, such as 25 μm to 250 μm, such as 30 μm to 200 μm, such as 35 μm to 150 μm and including 40 μm to 100 μm. In some cases, microwells have a pitch of 20 μm or more, such as 25 μm or more, such as 30 μm or more, such as 35 μm or more, such as 35 μm or more, such as 40 μm or more such as 45 μm or more, such as 49 μm or more, such as 50 μm or more, and including 55 μm or more. In one example, microwell arrays have 49 μm well pitch (39 μm well cross-section+10 μm wall separation).

A microwell array can comprise surface features between the microwells that are designed to help guide cells and solid supports into the wells and/or to prevent them from settling on the surfaces between wells. Non-limiting examples of suitable surface features include, but are not limited to, domed, ridged, or peaked surface features that encircle the wells or straddle the surface between wells.

A microwell can be fabricated using any of a number of fabrication techniques. Non-limiting examples of fabrication methods that can be used include bulk micromachining techniques such as photolithography and wet chemical etching, plasma etching, or deep reactive ion etching; micro-molding and micro-embossing; laser micromachining; 3D printing or other direct write fabrication processes using curable materials; and similar techniques.

Microwell arrays can be fabricated from a variety of substrate materials. The choice of material can depend on the choice of fabrication technique, and vice versa. Non-limiting examples of suitable materials include fused-silica, glass, polymers (e.g. agarose, gelatin, hydrogels, polydimethylsiloxane (PDMS) elastomer, polymethylmethacrylate (PMMA), polycarbonate (PC), polypropylene (PP), polyethylene (PE), high density polyethylene (HDPE), polyimide, cyclic olefin polymers (COP), cyclic olefin copolymers (COC), polyethylene terephthalate (PET), epoxy resins, thiol-ene based resins, metals or metal films (e.g. aluminum, stainless steel, copper, nickel, chromium, and titanium), and the like. A hydrophilic material can be desirable for fabrication of the microwell arrays (e.g. to enhance wettability and minimize non-specific binding of cells and other biological material). Hydrophobic materials that can be treated or coated (e.g., by oxygen plasma treatment, or grafting of a polyethylene oxide surface layer) can be used for fabrication of the microwell arrays. The use of porous, hydrophilic materials for the fabrication of the microwell array can be desirable in order to facilitate capillary wicking/venting of entrapped gas or air bubbles in the device. The microwell array can be fabricated from a single material. The microwell array can comprise two or more different materials that have been bonded together or mechanically joined.

A substrate can have variety of shapes and sizes. For example, the shape (or footprint) of the substrate within which microwells are fabricated can be square, rectangular, circular, or irregular in shape. The size of can be characterized by its width, length, and depth.

The thickness of the substrate within which the microwells are fabricated can range from about 0.1 mm thick to about 10 mm thick, or more. The thickness of the microwell array substrate can be, or be about, 0.1, 0.5, 1, 2, 3, 4, 5, 6, 7, 8, 9, 10 mm, or a number or a range between any two of these values. The thickness of the microwell array substrate can be at least, or at most, 10, 9, 8, 7, 6, 5, 4, 3, 2, 1, 0.5, 0.1 mm. The thickness of the microwell array substrate can be about 1 mm thick. The thickness of the microwell array substrate can be any value within these ranges, for example, the thickness of the microwell array substrate can be between about 0.2 mm and about 9.5 mm.

A variety of surface treatments and surface modification techniques can be used to modify the properties of microwell array surfaces. Examples can include, but are not limited to, oxygen plasma treatments to render hydrophobic material surfaces more hydrophilic, the use of wet or dry etching techniques to smooth or roughen glass and silicon surfaces, adsorption or grafting of polyethylene oxide or other polymer layers, for example pluronic, or bovine serum albumin to substrate surfaces to render them more hydrophilic and less prone to non-specific adsorption of biomolecules and cells, the use of silane reactions to graft chemically-reactive functional groups to otherwise inert silicon and glass surfaces, etc. Photodeprotection techniques can be used to selectively activate chemically-reactive functional groups at specific locations in the array structure, for example, the selective addition or activation of chemically-reactive functional groups such as primary amines or carboxyl groups on the inner walls of the microwells can be used to covalently couple oligonucleotide probes, peptides, proteins, or other biomolecules to the walls of the microwells. The choice of surface treatment or surface modification utilized can depend on the type of surface property that is desired and/or on the type of material from which the microwell array is made.

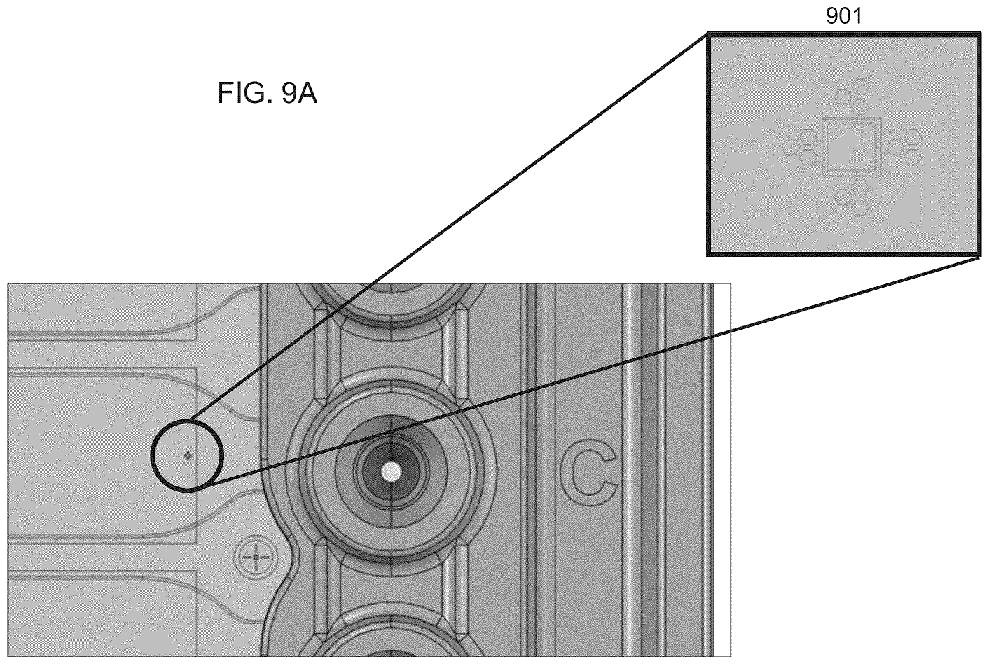

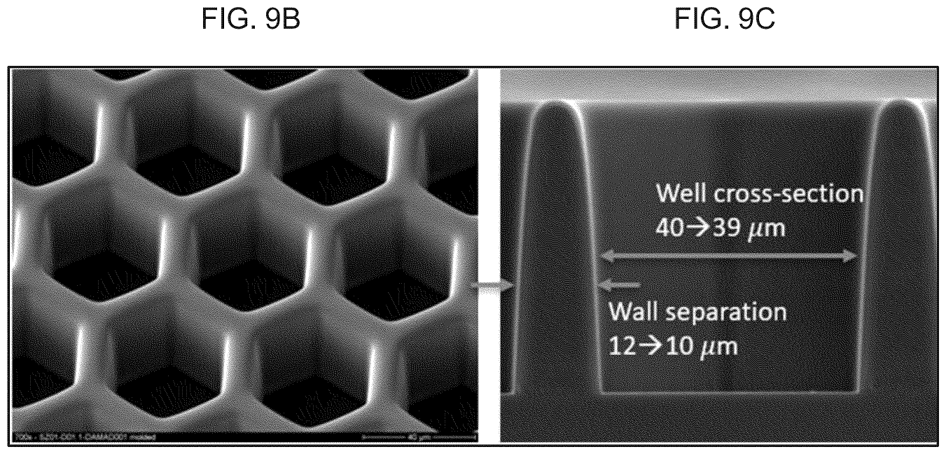

FIG. 9A-9C present different depictions of microwells of flowcells within cartridges of the invention. FIG. 9A depicts microwells 901 in a flowcell of a multi-microwell-array-flowcell cartridge according to certain embodiments. As shown in FIG. 9A, the microwell-array includes hexagonal microwells. FIG. 9B provides a more-detailed depiction of the hexagonal microwells shown in FIG. 9A, and FIG. 9C depicts pitch measurement according to one embodiment of the invention. As shown in FIG. 9C, the wells have 49 μm well pitch (39 μm well cross-section+10 μm wall separation).

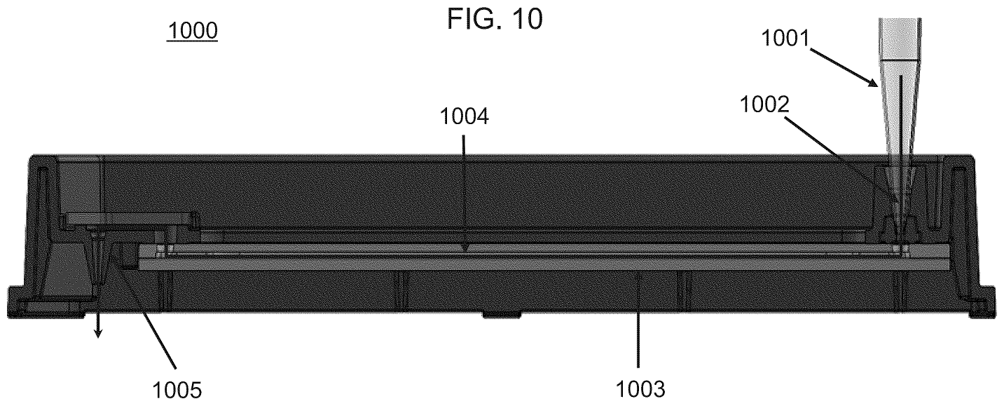

FIG. 10 depicts fluid flow through a multi-microwell-array-flowcell cartridge according to certain embodiments. Liquid is introduced into cartridge 1000 via pipette 1001 using inlet 1002. The liquid follows fluid path 1004 and passes through the elongate channel 1003 of a flowcell. The flowcell comprises a microwell array, such as the one discussed with respect to FIG. 9A-9C. Single cells may be captured within the wells. Liquid path 1004 proceeds to outlet 1005, which is shown as a stepped outlet including a raised portion that fluid must travel over prior to being discharged. This prevents siphoning of the liquid.

### Cartridges

The cartridge can further comprise components that are designed to create physical or chemical barriers that prevent diffusion of (or increase path lengths and diffusion times for) large molecules in order to minimize cross-contamination between microwells. Examples of such barriers can include, but are not limited to, a pattern of serpentine channels used for delivery of cells and solid supports (e.g., beads) to the microwell array, a retractable platen or deformable membrane that is pressed into contact with the surface of the microwell array substrate during lysis or incubation steps, the use of larger beads, e.g. Sephadex beads as described previously, to block the openings of the microwells, or the release of an immiscible, hydrophobic fluid from a reservoir within the cartridge during lysis or incubation steps, to effectively separate and compartmentalize each microwell in the array.

Cartridges can be fabricated using a variety of techniques and materials known to those of skill in the art. In general, the cartridges will be fabricated as a series of separate component parts and subsequently assembled using any of a number of mechanical assemblies or bonding techniques. Examples of suitable fabrication techniques include, but are not limited to, conventional machining, CNC machining, injection molding, thermoforming, and 3D printing. Once the cartridge components have been fabricated they can be mechanically assembled using screws, clips, and the like, or permanently bonded using any of a variety of techniques (depending on the choice of materials used), for example, through the use of thermal bonding/welding or any of a variety of adhesives or adhesive films, including epoxy-based, acrylic-based, silicone-based, UV curable, polyurethane-based, or cyanoacrylate-based adhesives.

Cartridge components can be fabricated using any of a number of suitable materials, including but not limited to silicon, fused-silica, glass, any of a variety of polymers, e.g. polydimethylsiloxane (PDMS; elastomer), polymethylmethacrylate (PMMA), polycarbonate (PC), polypropylene (PP), polyethylene (PE), high density polyethylene (HDPE), polyimide, cyclic olefin polymers (COP), cyclic olefin copolymers (COC), polyethylene terephthalate (PET), epoxy resins, non-stick materials such as teflon (PTFE), metals (e.g. aluminum, stainless steel, copper, nickel, chromium, and titanium), or any combination thereof.

The cartridge can include integrated miniature pumps or other fluid actuation mechanisms for control of fluid flow through the device. Examples of suitable miniature pumps or fluid actuation mechanisms can include, but are not limited to, electromechanically- or pneumatically-actuated miniature syringe or plunger mechanisms, membrane diaphragm pumps actuated pneumatically or by an external piston, pneumatically-actuated reagent pouches or bladders, or electro-osmotic pumps.

The cartridge can include miniature valves for compartmentalizing pre-loaded reagents or controlling fluid flow through the device. Examples of suitable miniature valves can include, but are not limited to, one-shot “valves” fabricated using wax or polymer plugs that can be melted or dissolved, or polymer membranes that can be punctured; pinch valves constructed using a deformable membrane and pneumatic, magnetic, electromagnetic, or electromechanical (solenoid) actuation, one-way valves constructed using deformable membrane flaps, and miniature gate valves.

The cartridge can include vents for providing an escape path for trapped air or gas such as CO₂ or N₂. Vents can be constructed according to a variety of techniques, for example, using a porous plug of polydimethylsiloxane (PDMS) or other hydrophobic material that allows for capillary wicking of air or gas but blocks penetration by water.

The mechanical interface features of the cartridge can provide for easily removable but highly precise and repeatable positioning of the cartridge relative to the instrument system. Suitable mechanical interface features can include, but are not limited to, alignment pins, alignment guides, mechanical stops, and the like. The mechanical design features can include relief features for bringing external apparatus, e.g. magnets or optical components, into close proximity with the microwell array chamber.

The cartridge can include temperature control components or thermal interface features for mating to external temperature control modules. Examples of suitable temperature control elements can include, but are not limited to, resistive heating elements, miniature infrared-emitting light sources, Peltier heating or cooling devices, heat sinks, thermistors, thermocouples, and the like. Thermal interface features can be fabricated from materials that are good thermal conductors (e.g. copper, gold, silver, etc.) and can comprise one or more flat surfaces capable of making good thermal contact with external heating blocks or cooling blocks.

The cartridge can include optical interface features for use in optical imaging or spectroscopic interrogation of the microwell array. The cartridge can include an optically transparent window, e.g. the microwell substrate itself or the side of the flowcell or microarray chamber that is opposite the microwell array, fabricated from a material that meets the spectral requirements for the imaging or spectroscopic technique used to probe the microwell array. Examples of suitable optical window materials can include, but are not limited to, glass, fused-silica, polymethylmethacrylate (PMMA), polycarbonate (PC), cyclic olefin polymers (COP), or cyclic olefin copolymers (COC).

### Cartridge Carrier

Embodiments of the invention may also include a carrier for the cartridge. The carrier may be used for, e.g., transport of the cartridge. In some cases, the cartridge carrier is configured to support the imaging of the cartridge/flowcell contents in any of the microwell lanes during an experiment. In certain instances, the cartridge includes one or more kinematic mounts configured to orient the cartridge carrier—and the cartridge, when such is placed in the cartridge carrier—within an analyzer (e.g., the BD Rhapsody™ Scanner). In some such instances, the number of kinematic mounts in the cartridge carrier may range from 1 to 5, and including 2 to 4. In select cases, the cartridge carrier has 3 kinematic mounts. The cartridge carrier may include one or more relief portions. The relief portions may be used for the installation or removal of the cartridge from the holder. In some cases, the cartridge carrier includes a chamfered corner so that the cartridge may be placed in the carrier in a single orientation. In certain versions, the cartridge carrier includes a drip receptacle configured to sequester liquid drips emanating from the outlets of the cartridge.

The cartridge carrier may be configured for use with a single lane cartridge (e.g., such as those described in U.S. Pat. Nos. 10,634,691 and 11,061,043), or the multi-microwell-array-flowcell cartridge of the invention. In some cases, the cartridge carrier may be configured for use with a single lane cartridge. In such cases, the cartridge carrier may include a drip receptacle positioned to sequester liquid drips emanating from the single outlet of the of the single lane cartridge. In other cases, the cartridge carrier may be configured for use with the multi-microwell-array-flowcell cartridge of the invention. In such cases, the cartridge carrier may include a drip receptacle positioned to sequester liquid drips emanating from each outlet of the multi-microwell-array-flowcell cartridge of the invention. In still other cases, cartridge carriers are configured for use with both the single lane cartridge and the multi-microwell-array-flowcell cartridge of the invention. In some such cases, the cartridge carrier includes both a drip receptacle positioned to sequester liquid drips emanating from each outlet of the multi-microwell-array-flowcell cartridge of the invention, and a drip receptacle positioned to sequester liquid drips emanating from the single outlet of the of the single lane cartridge.

The cartridge carrier may be constructed from any convenient material. In some instances, the cartridge carrier is constructed from a metal material, including but not limited to aluminum, titanium, brass, iron, lead, nickel, steel (e.g., stainless steel), copper, tin as well as combinations and alloys thereof. In select versions, the cartridge carrier is constructed from 6061-T6 Aluminum and hard anodized with class III PTFE impregnated.

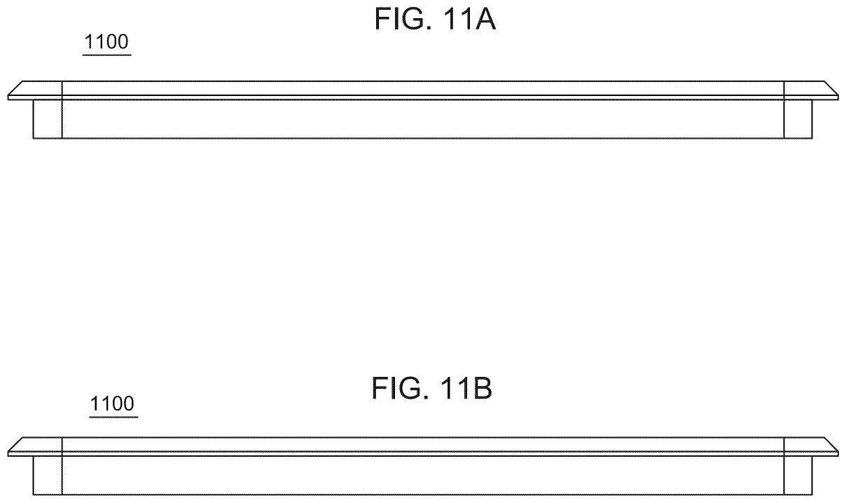

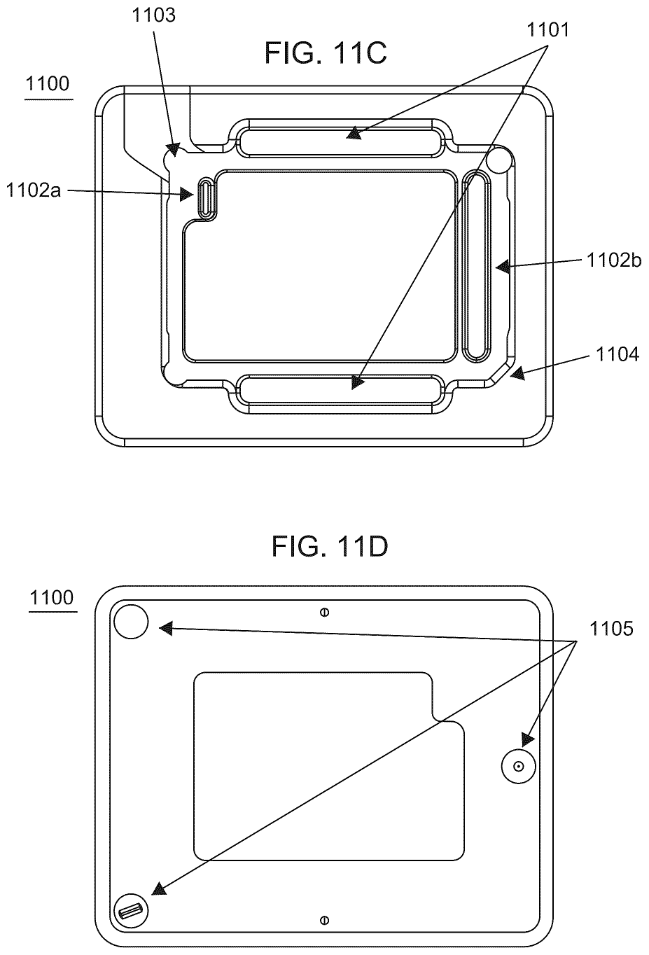

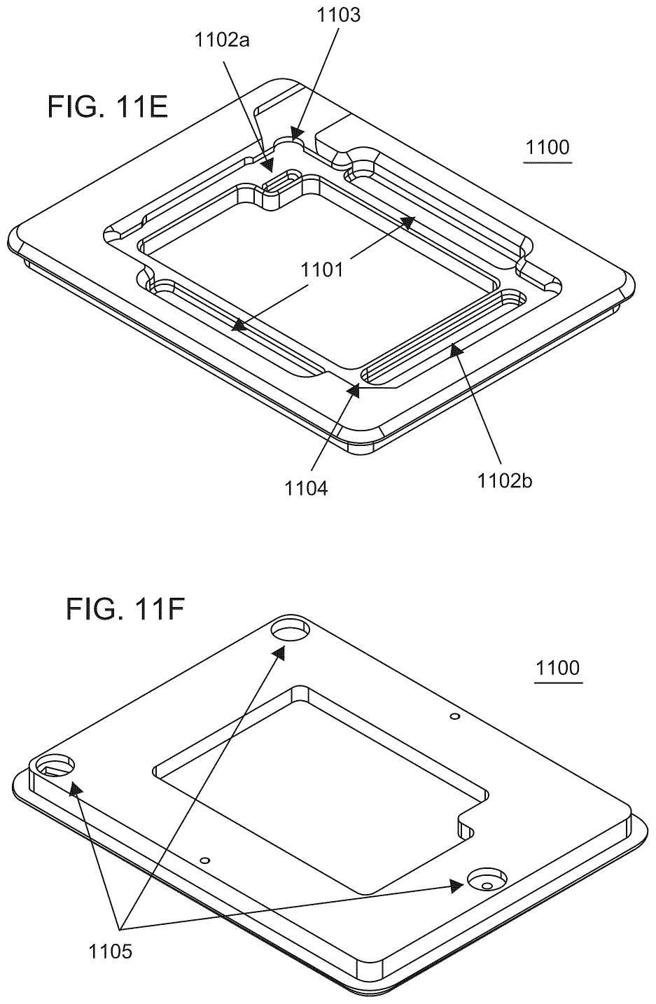

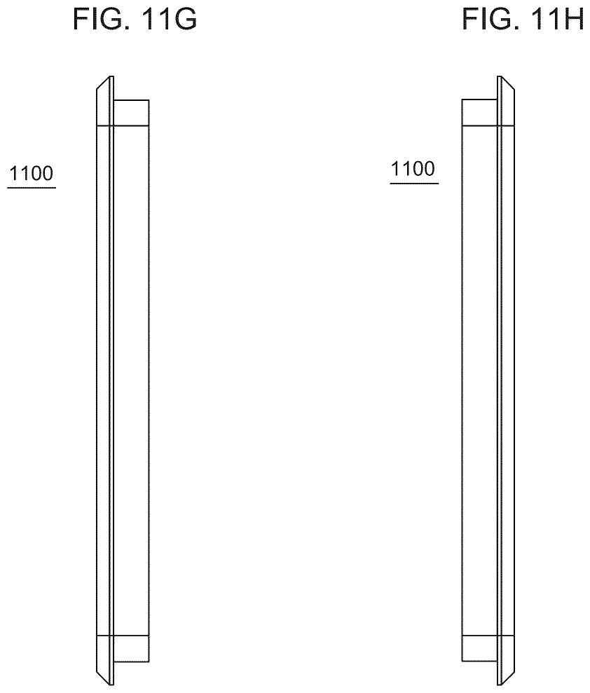

A cartridge carrier of the invention is shown in FIG. 11A-11H. FIG. 11A-11B show different side profile views of cartridge carrier 1100. FIG. 11C presents a top view of cartridge carrier 1100. As shown in FIG. 11C, cartridge 1100 includes relief portions 1101 configured for the installation or removal of the cartridge from the holder. In addition, cartridge 1100 includes drip receptacles 1102a-b. Drip receptacle 1102a is positioned to sequester liquid drips emanating from the single outlet of the of the single lane cartridge, while drip receptacle 1102b is positioned to sequester liquid drips emanating from each outlet of the multi-microwell-array-flowcell cartridge of the invention. Also shown is cartridge bias relief 1103. and chamfered corner 1104. FIG. 11D presents a bottom view of cartridge holder 1100 where kinematic mounts 1105 are visible. FIG. 11E-11F show 45° angle shots of cartridge carrier 1100, while FIG. 11G-11H show side views of cartridge carrier 1100

### Sample Collection Vessel Holders

As discussed above, aspects of the invention also include a sample vessel holder. Sample vessel holders of interest are configured to receive a plurality of sample collection vessels for collecting an analyte from the multi-microwell-array-flowcell cartridge (e.g., described above). The sample collection vessel holder may be configured to contain any suitable number of sample collection vessels, such as where the number of sample collection vessels ranges from 2 to 10, including 7 to 9. In some cases, the system comprises 8 sample collection vessels. In select embodiments, the sample collection vessel holder comprises a counterweight configured to maintain the sample collection holder in an upright position. In certain embodiments, the sample collection vessel holder has a complementary shape to a cellular analysis system such that the sample collection vessel holder may be received in the cellular analysis system in a single orientation. In other words, the sample collection vessel holder may have a poka-yoke design. In some cases, the sample collection vessel holder has a rounded end.

The sample vessel holder may be constructed from any of a number of suitable materials, including but not limited to silicon, fused-silica, glass, any of a variety of polymers, e.g. polydimethylsiloxane (PDMS; elastomer), polymethylmethacrylate (PMMA), polycarbonate (PC), polypropylene (PP), polyethylene (PE), high density polyethylene (HDPE), polyimide, cyclic olefin polymers (COP), cyclic olefin copolymers (COC), polyethylene terephthalate (PET), epoxy resins, non-stick materials such as teflon (PTFE), metals (e.g. aluminum, stainless steel, copper, nickel, chromium, and titanium), or any combination thereof.

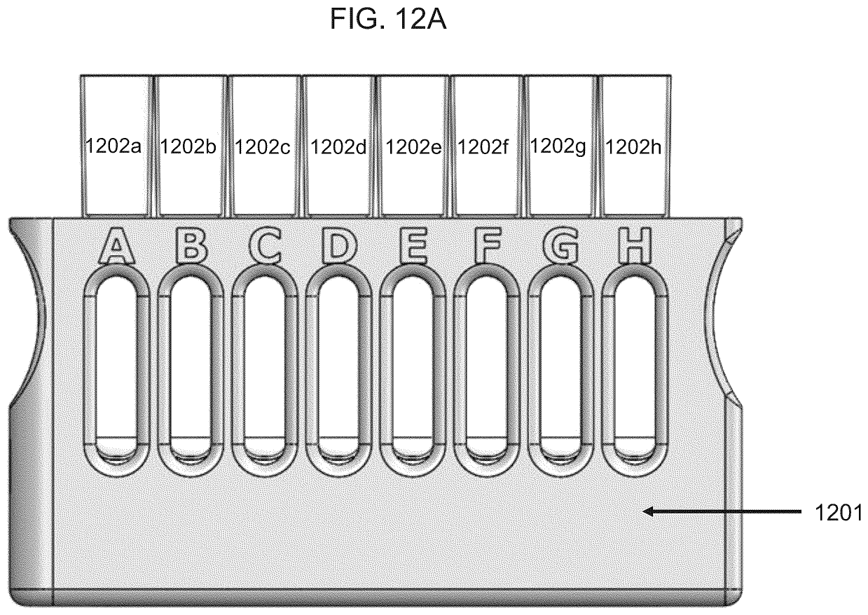

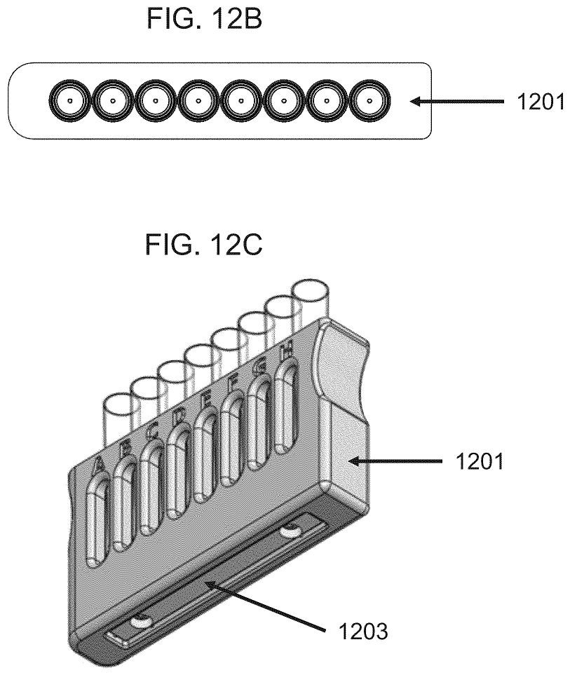

FIG. 12A-12C depict a sample collection vessel holder of the invention according to certain embodiments. As shown in FIG. 12A, sample collection vessel holder 1201 is configured to hold 8 sample collection vessels 1202a-1202h, which are depicted as test tubes. FIG. 12B presents a view from the top of sample collection vessel holder 1201. As shown by its rounded edge, sample collection vessel holder 1201 is shaped such that it can only be inserted in a drawer of a system of the invention in a single orientation. As shown in FIG. 12C, sample collection vessel holder 1201 comprises a counterweight 1203 configured to maintain the sample collection holder in an upright position.

### Methods for Processing a Cellular Sample

As discussed above, aspects of the invention include methods of processing a cellular sample. Methods of interest include introducing a multi-microwell-array-flowcell cartridge into a system comprising a tray configured to receive the multi-microwell-array-flowcell cartridge, and a retrieval magnet assembly configured to apply a uniform magnetic force to the flowcells of the multi-microwell-array-flowcell cartridge when in an active position. Methods also include loading a sample into the multi-microwell-array-flowcell cartridge, actuating the retrieval magnet assembly to the active position and applying the uniform magnetic force to the sample within the multi-microwell-array-flowcell cartridge to produce a processed sample, and collecting the processed sample from the multi-microwell-array-flowcell cartridge.

In certain cases, the method comprises loading a plurality of different samples into the different flowcells of the cartridge. The number of samples may vary and can range from, in some cases, from 2 to 10 (e.g., 8). Methods according to certain embodiments include loading a lysis buffer into the multi-well cartridge after the loading of the sample liquid into the multi-well cartridge. In some versions, methods include loading the lysis buffer into the multi-well cartridge without engaging a lysis magnet.

Methods according to select versions of the invention include loading barcoded beads into the multi-well cartridge prior to the actuation of the retrieval magnet assembly. The barcoded beads may, in certain cases, include a nucleic acid barcode comprising a universal primer binding domain, a cell label domain and a target capture domain. In select embodiments, the target capture domain is an poly(T) sequence. In certain instances, the nucleic acid barcode further comprises a unique molecular index (UMI).

In certain cases, methods include applying the uniform magnetic from a position that is superior to the tray. As discussed above, systems that may be used in the subject methods include a tray configured to receive a multi-microwell-array-flowcell cartridge, and a retrieval magnet assembly configured to apply a uniform magnetic force to flowcells of the cartridge when in an active position. In some cases, the retrieval magnet assembly is configured to apply the uniform magnetic force from a position that is superior to the tray. The uniform magnetic force may, in certain cases, be a magnetic field ranging from 650 Gauss to 1325 Gauss. In certain instances, the retrieval magnet assembly is configured to apply the uniform magnetic force via a plurality of magnets. In some such instances, the plurality of magnets have alternating polarities. The number of magnets in the retrieval magnet assembly may vary, and in some cases range from 1 to 10 (e.g., 4). The type of magnets in the retrieval magnet assembly may also vary. In some instances, the magnets are rare earth magnets (e.g., neodymium magnets and/or samarium-cobalt magnets). The shape of the magnets may also vary. In some cases, the magnets of the plurality are bar magnets. In other cases, the magnets of the plurality are ring magnets. In some such cases, magnets of the plurality are arranged in a bullseye configuration. Systems of the invention may be configured to adjust the position of the retrieval magnet assembly to process the cellular sample. In some embodiments, the retrieval magnet assembly is actuatable between the active position in which the retrieval magnet assembly is positioned adjacent to the tray, and an inactive position in which the retrieval magnet is located at a further distance from the tray relative to the active position.

In some versions, methods include the use of a sample collection vessel holder configured to receive a plurality of sample collection vessels for collecting an analyte from the cartridge. In some such versions, the sample collection vessel holder comprises a counterweight configured to maintain the sample collection holder in an upright position. The system and the sample collection vessel holder may also have complementary shapes such that the sample collection vessel holder may be received in the system in a single orientation. In some aspects, systems also include the plurality sample collection vessels. The number of sample collection vessels may vary, and can range in some cases from 2 to 10 (e.g., 8). In certain instances, the system comprises a number of sample collection vessels that matches the number of flowcells in the cartridge. In some embodiments, systems of the invention include a waste collection vessel for collecting liquid waste from the multi-microwell-array-flowcell cartridge. In some instances, methods include an interlock configured to prevent the collection of sample liquid into the waste collection vessel when the retrieval magnet assembly is in the active position. In some cases, the tray comprises a latch for retaining the multi-microwell-array-flowcell cartridge. In select versions, systems do not include a lysis magnet at an inferior position to the tray.

As noted above, methods of the invention include the use of a multi-microwell-array-flowcell cartridge. The multi-microwell-array-flowcell cartridge for use in the methods includes a plurality of fluidic lanes, each fluidic lane comprising an inlet for receiving liquid, a flow cell comprising a microwell array, and an outlet for discharging liquid. In certain cases, the multi-microwell-array-flowcell cartridge comprises a number of fluidic lanes ranging from 2 to 10 (e.g., 8). In some versions, each outlet is stepped to prevent siphoning of liquid from the flow cell. In some embodiments, each flow cell is comprised of an elongate channel. The length of the elongate channel may vary, and can range in some cases from 50 mm to 100 mm. The number of microwells in each microwell array may also vary, and can range, in some cases, from 250,000 microwells to 300,000 microwells. The density of microwells within each microwell may vary, and can range in some cases from 36,000 microwells/cm² to 42,000 microwells/cm².

The type of analysis performed in the subject methods may vary, as desired. In some cases, the method comprises producing a sequence ready nucleic acid library from the processed sample. In certain embodiments, the sequence ready nucleic acid library is sequence-able by using a next generation sequencing protocol. In additional embodiments, the method is a method of genomic analysis, epigenomic analysis, transcriptomic analysis or proteomic analysis. In select versions, the method is a method of multiomic analysis, such as where the multiomic analysis comprises at least transcriptomic and proteomic analysis.

### Stochastic Barcodes

In some embodiments, the subject methods include barcoding (e.g., stochastic barcoding). Stochastic barcoding has been described in, for example, US20150299784, WO2015031691, and Fu et al, Proc Natl Acad Sci U.S.A. 2011 May 31; 108(22):9026-31, the content of these publications is incorporated hereby in its entirety. Briefly, a stochastic barcode can be a polynucleotide sequence that may be used to stochastically label (e.g., barcode, tag) a target. A stochastic barcode can comprise one or more labels. Exemplary labels can include a universal label, a cellular label, a molecular label, a sample label, a plate label, a spatial label, and/or a pre-spatial label. The stochastic barcode can comprise a 5′amine that may link the stochastic barcode to a solid support.

The stochastic barcode can comprise a universal label, a dimension label, a spatial label, a cellular label, and/or a molecular label. The order of different labels (including but not limited to the universal label, the dimension label, the spatial label, the cellular label, and the molecule label) in the stochastic barcode can vary. For example, the universal label may be the 5′-most label, and the molecular label may be the 3′-most label. The spatial label, dimension label, and the cellular label may be in any order. In some embodiments, the universal label, the spatial label, the dimension label, the cellular label, and the molecular label are in any order.

The stochastic barcodes can be from a “non-depleting reservoir,” a pool of stochastic barcodes made up of many different labels. A non-depleting reservoir can comprise large numbers of different stochastic barcodes such that when the non-depleting reservoir is associated with a pool of targets each target is likely to be associated with a unique stochastic barcode. The uniqueness of each labeled target molecule can be determined by the statistics of random choice, and depends on the number of copies of identical target molecules in the collection compared to the diversity of labels. The size of the resulting set of labeled target molecules can be determined by the stochastic nature of the barcoding process, and analysis of the number of stochastic barcodes detected then allows calculation of the number of target molecules present in the original collection or sample. When the ratio of the number of copies of a target molecule present to the number of unique stochastic barcodes is low, the labeled target molecules are highly unique (i.e., there is a very low probability that more than one target molecule will have been labeled with a given label).

A label, for example the cellular label, can comprise a unique set of nucleic acid sub-sequences of defined length, e.g., seven nucleotides each (equivalent to the number of bits used in some Hamming error correction codes), which can be designed to provide error correction capability. The set of error correction sub-sequences comprise seven nucleotide sequences can be designed such that any pairwise combination of sequences in the set exhibits a defined “genetic distance” (or number of mismatched bases), for example, a set of error correction sub-sequences can be designed to exhibit a genetic distance of three nucleotides. In this case, review of the error correction sequences in the set of sequence data for labeled target nucleic acid molecules (described more fully below) can allow one to detect or correct amplification or sequencing errors. In some embodiments, the length of the nucleic acid sub-sequences used for creating error correction codes can vary, for example, they can be, or be about 1, 2, 3, 4, 5, 6, 7, 8, 9, 10, 15, 20, 30, 31, 40, 50, or a number or a range between any two of these values, nucleotides in length. In some embodiments, nucleic acid sub-sequences of other lengths can be used for creating error correction codes.

The stochastic barcode can comprise a target-binding region. The target-binding region can interact with a target in a sample. The target can be, or comprise, ribonucleic acids (RNAs), messenger RNAs (mRNAs), microRNAs, small interfering RNAs (siRNAs), RNA degradation products, RNAs each comprising a poly(A) tail, and any combination thereof. In some embodiments, the plurality of targets can include deoxyribonucleic acids (DNAs).

In some embodiments, a target-binding region can comprise an oligo(dT) sequence which can interact with poly(A) tails of mRNAs. One or more of the labels of the stochastic barcode (e.g., the universal label, the dimension label, the spatial label, the cellular label, and the molecular label) can be separated by a spacer from another one or two of the remaining labels of the stochastic barcode. The spacer can be, for example, 1, 2, 3, 4, 5, 6, 7, 8, 9, 10, 11, 12, 13, 14, 15, 16, 17, 18, 19, or 20 or more nucleotides. In some embodiments, none of the labels of the stochastic barcode is separated by spacer.

A stochastic barcode can comprise one or more universal labels, one or more dimension labels, one or more spatial labels, one or more cellular labels, one or more molecular labels, one or more target binding regions, or any combination thereof.

The one or more universal labels can be the same for all stochastic barcodes in the set of stochastic barcodes attached to a given solid support (e.g., beads), or the same for all stochastic barcodes attached to a plurality of beads. A universal label can comprise a nucleic acid sequence that is capable of hybridizing to a sequencing primer. A universal label can comprise a nucleic acid sequence that is capable of hybridizing to a PCR primer, or comprise a nucleic acid sequence that is capable of hybridizing to a sequencing primer and a PCR primer. The nucleic acid sequence of the universal label that is capable of hybridizing to a sequencing or PCR primer can be referred to as a primer binding site. A universal label can comprise a sequence that can be used to initiate transcription of the stochastic barcode. A universal label can comprise a sequence that can be used for extension of the stochastic barcode or a region within the stochastic barcode.

A dimension label can comprise a nucleic acid sequence that provides information about a dimension in which the stochastic labeling occurred. For example, a dimension label can provide information about the time at which a target was stochastically barcoded. A dimension label can be associated with a time of stochastic barcoding in a sample. A dimension label can be activated at the time of stochastic labeling. Different dimension labels can be activated at different times. The dimension label provides information about the order in which targets, groups of targets, and/or samples were stochastically barcoded. For example, a population of cells can be stochastically barcoded at the G0 phase of the cell cycle. The cells can be pulsed again with stochastic barcodes at the G1 phase of the cell cycle. The cells can be pulsed again with stochastic barcodes at the S phase of the cell cycle, and so on. Stochastic barcodes at each pulse (e.g., each phase of the cell cycle), can comprise different dimension labels. In this way, the dimension label provides information about which targets were labeled at which phase of the cell cycle. Dimension labels can interrogate many different biological times. Exemplary biological times can include, but are not limited to, the cell cycle, transcription (e.g., transcription initiation), and transcript degradation. In another example, a sample (e.g., a cell, a population of cells) can be stochastically labeled before and/or after treatment with a drug and/or therapy. The changes in the number of copies of distinct targets can be indicative of the sample's response to the drug and/or therapy.

### Solid Supports

Stochastic barcodes disclosed herein can, in some embodiments, be associated with a solid support. The solid support can be, for example, a synthetic particle. In some embodiments, some or all of the molecular labels (e.g., the first molecular labels) of a plurality of stochastic barcodes (e.g., the first plurality of stochastic barcodes) on a solid support differ by at least one nucleotide. The cellular labels of the stochastic barcodes on the same solid support can be the same. The cellular labels of the stochastic barcodes on different solid supports can differ by at least one nucleotide. For example, first cellular labels of a first plurality of stochastic barcodes on a first solid support can have the same sequence, and second cellular labels of a second plurality of stochastic barcodes on a second solid support can have the same sequence. The first cellular labels of the first plurality of stochastic barcodes on the first solid support and the second cellular labels of the second plurality of stochastic barcodes on the second solid support can differ by at least one nucleotide. A cellular label can be, for example, about 5-20 nucleotides long. A molecular label can be, for example, about 5-20 nucleotides long.

The synthetic particle can be, for example, a bead. The bead can be, for example, a silica gel bead, a controlled pore glass bead, a magnetic bead, a Dynabead, a Sephadex/Sepharose bead, a cellulose bead, a polystyrene bead, or any combination thereof. The bead can comprise a material such as polydimethylsiloxane (PDMS), polystyrene, glass, polypropylene, agarose, gelatin, hydrogel, paramagnetic, ceramic, plastic, glass, methylstyrene, acrylic polymer, titanium, latex, sepharose, cellulose, nylon, silicone, or any combination thereof.

For example, after introducing cells such as single cells onto a plurality of microwells of a microwell array, beads can be introduced onto the plurality of microwells of the microwell array. Each microwell can comprise one bead. The beads can comprise a plurality of stochastic barcodes. A stochastic barcode can comprise a 5′ amine region attached to a bead. The stochastic barcode can comprise a universal label, a molecular label, a target-binding region, or any combination thereof.

The stochastic barcodes disclosed herein can be associated to (e.g., attached to) a solid support (e.g., a bead). The stochastic barcodes associated with a solid support can each comprise a molecular label selected from a group comprising at least 100 or 1000 molecular labels with unique sequences. In some embodiments, different stochastic barcodes associated with a solid support can comprise molecular labels of different sequences. In some embodiments, a percentage of stochastic barcodes associated with a solid support comprises the same cell label. For example, the percentage can be, or be about 60%, 70%, 80%, 85%, 90%, 95%, 97%, 99%, 100%, or a number or a range between any two of these values. As another example, the percentage can be at least, or at most 60%, 70%, 80%, 85%, 90%, 95%, 97%, 99%, or 100%. In some embodiments, stochastic barcodes associated with a solid support can have the same cell label. The stochastic barcodes associated with different solid supports can have different cell labels selected from a group comprising at least 100 or 1000 cell labels with unique sequences.

In some cases, the barcoded beads comprise a nucleic acid barcode comprising a universal primer binding domain, a cell label domain and a target capture domain. In select cases, the target capture domain is an poly(T) sequence. In certain versions, the nucleic acid barcode further comprises a unique molecular index (UMI). Stochastic barcodes with unique molecular labels (also referred to as molecular indexes (MIs)) can be used to count the number of molecules and correct for amplification bias. Stochastic barcoding such as the Precise™ assay (Cellular Research, Inc. (Palo Alto, Calif.)) can correct for bias induced by PCR and library preparation steps by using molecular labels (MLs) to label mRNAs during reverse transcription (RT). In some embodiments, methods include using a non-depleting pool of stochastic barcodes with large number, for example 6561 to 65536, unique molecular labels on poly(T) oligonucleotides to hybridize to all poly(A)-mRNAs in a sample during the RT step. A stochastic barcode can comprise a universal PCR priming site. During RT, target gene molecules react randomly with stochastic barcodes. Each target molecule can hybridize to a stochastic barcode resulting to generate stochastically barcoded complementary ribonucleotide acid (cDNA) molecules). After labeling, stochastically barcoded cDNA molecules from microwells of a microwell plate can be pooled into a single tube for PCR amplification and sequencing. Raw sequencing data can be analyzed to produce the number of reads, the number of stochastic barcodes with unique molecular labels, and the numbers of mRNA molecules.

In some embodiments, stochastically barcoding the plurality of targets in the sample can be performed with a solid support including a plurality of synthetic particles associated with the plurality of stochastic barcodes. In some embodiments, the solid support can include a plurality of synthetic particles associated with the plurality of stochastic barcodes. The spatial labels of the plurality of stochastic barcodes on different solid supports can differ by at least one nucleotide. The solid support can, for example, include the plurality of stochastic barcodes in two dimensions or three dimensions. The synthetic particles can be beads. The beads can be silica gel beads, controlled pore glass beads, magnetic beads, Dynabeads, Sephadex/Sepharose beads, cellulose beads, polystyrene beads, or any combination thereof. The solid support can include a polymer, a matrix, a hydrogel, a needle array device, an antibody, or any combination thereof. In some embodiments, the solid supports can be free floating. In some embodiments, the solid supports can be embedded in a semi-solid or solid array. The stochastic barcodes may not be associated with solid supports. The stochastic barcodes can be individual nucleotides. The stochastic barcodes can be associated with a substrate.

As used herein, the terms “tethered”, “attached”, and “immobilized” are used interchangeably, and can refer to covalent or non-covalent means for attaching stochastic barcodes to a solid support. Any of a variety of different solid supports can be used as solid supports for attaching pre-synthesized stochastic barcodes or for in situ solid-phase synthesis of stochastic barcode.

In some embodiments, the solid support is a bead. The bead can comprise one or more types of solid, porous, or hollow sphere, ball, bearing, cylinder, or other similar configuration which a nucleic acid can be immobilized (e.g., covalently or non-covalently). The bead can be, for example, composed of plastic, ceramic, metal, polymeric material, or any combination thereof. A bead can be, or comprise, a discrete particle that is spherical (e.g., microspheres) or have a non-spherical or irregular shape, such as cubic, cuboid, pyramidal, cylindrical, conical, oblong, or disc-shaped, and the like. In some embodiments, a bead can be non-spherical in shape.

Beads can comprise a variety of materials including, but not limited to, paramagnetic materials (e.g. magnesium, molybdenum, lithium, and tantalum), superparamagnetic materials (e.g. ferrite (Fe₃O₄; magnetite) nanoparticles), ferromagnetic materials (e.g. iron, nickel, cobalt, some alloys thereof, and some rare earth metal compounds), ceramic, plastic, glass, polystyrene, silica, methylstyrene, acrylic polymers, titanium, latex, sepharose, agarose, hydrogel, polymer, cellulose, nylon, and any combination thereof. In some embodiments, the bead (e.g., the bead to which the stochastic labels are attached) is a hydrogel bead. In some embodiments, the bead comprises hydrogel.

The size of the beads can vary. For example, the diameter of the bead can range from 0.1 micrometer to 50 micrometers. In some embodiments, the diameters of beads can be, or be about, 0.1, 0.5, 1, 2, 3, 4, 5, 6, 7, 8, 9, 10, 20, 30, 40, 50 micrometers, or a number or a range between any two of these values.

The diameters of the bead can be related to the diameter of the wells of the substrate. In some embodiments, the diameters of the bead can be, or be about, 10%, 20%, 30%, 40%, 50%, 60%, 70%, 80%, 90%, 100%, or a number or a range between any two of these values, longer or shorter than the diameter of the well. The diameter of the beads can be related to the diameter of a cell (e.g., a single cell entrapped by a well of the substrate). In some embodiments, the diameters of the beads can be, or be about, 10%, 20%, 30%, 40%, 50%, 60%, 70%, 80%, 90%, 100%, 150%, 200%, 250%, 300%, or a number or a range between any two of these values, longer or shorter than the diameter of the cell.

A bead can be attached to and/or embedded in a substrate. A bead can be attached to and/or embedded in a gel, hydrogel, polymer and/or matrix. The spatial position of a bead within a substrate (e.g., gel, matrix, scaffold, or polymer) can be identified using the spatial label present on the stochastic barcode on the bead which can serve as a location address.

Examples of beads can include, but are not limited to, streptavidin beads, agarose beads, magnetic beads, Dynabeads®, MACS® microbeads, antibody conjugated beads (e.g., anti-immunoglobulin microbeads), protein A conjugated beads, protein G conjugated beads, protein A/G conjugated beads, protein L conjugated beads, oligo(dT) conjugated beads, silica beads, silica-like beads, anti-biotin microbeads, anti-fluorochrome microbeads, and BcMag™ Carboxyl-Terminated Magnetic Beads.

A bead can be associated with (e.g. impregnated with) quantum dots or fluorescent dyes to make it fluorescent in one fluorescence optical channel or multiple optical channels. A bead can be associated with iron oxide or chromium oxide to make it paramagnetic or ferromagnetic. Beads can be identifiable. For example, a bead can be imaged using a camera. A bead can have a detectable code associated with the bead. For example, a bead can comprise a stochastic barcode. A bead can change size, for example due to swelling in an organic or inorganic solution. A bead can be hydrophobic. A bead can be hydrophilic. A bead can be biocompatible.

A solid support (e.g., bead) can be visualized. The solid support can comprise a visualizing tag (e.g., fluorescent dye). A solid support (e.g., bead) can be etched with an identifier (e.g., a number). The identifier can be visualized through imaging the beads.

### Methods of Stochastic Barcoding

Provided herein are methods for estimating the number of distinct targets at distinct locations in a physical sample (e.g., tissue, organ, tumor, cell). The methods can comprise placing the stochastic barcodes in close proximity with the sample, lysing the sample, associating distinct targets with the stochastic barcodes, amplifying the targets and/or digitally counting the targets. The method can further comprise analyzing and/or visualizing the information obtained from the spatial labels on the stochastic barcodes. In some embodiments, the method comprises visualizing the plurality of targets in the sample. Mapping the plurality of targets onto the map of the sample can include generating a two dimensional map or a three dimensional map of the sample. The two dimensional map and the three dimensional map can be generated prior to or after stochastically barcoding the plurality of targets in the sample. Visualizing the plurality of targets in the sample can include mapping the plurality of targets onto a map of the sample. Mapping the plurality of targets onto the map of the sample can include generating a two dimensional map or a three dimensional map of the sample. The two dimensional map and the three dimensional map can be generated prior to or after stochastically barcoding the plurality of targets in the sample. In some embodiments, the two dimensional map and the three dimensional map can be generated before or after lysing the sample. Lysing the sample before or after generating the two dimensional map or the three dimensional map can include heating the sample, contacting the sample with a detergent, changing the pH of the sample, or any combination thereof.

In some embodiments, stochastically barcoding the plurality of targets comprises hybridizing a plurality of stochastic barcodes with a plurality of targets to create stochastically barcoded targets. Stochastically barcoding the plurality of targets can comprise generating an indexed library of the stochastically barcoded targets.

Generating an indexed library of the stochastically barcoded targets can be performed with a solid support comprising the plurality of stochastic barcodes.

The disclosure provides for methods for contacting a sample (e.g., cells) to a substrate of the disclosure. A sample comprising, for example, a cell, organ, or tissue thin section, can be contacted to stochastic barcodes. The cells can be contacted, for example, by gravity flow wherein the cells can settle and create a monolayer. The sample can be a tissue thin section. The thin section can be placed on the substrate. The sample can be one-dimensional (e.g., form a planar surface). The sample (e.g., cells) can be spread across the substrate, for example, by growing/culturing the cells on the substrate.

When stochastic barcodes are in close proximity to targets, the targets can hybridize to the stochastic barcode. The stochastic barcodes can be contacted at a non-depletable ratio such that each distinct target can associate with a distinct stochastic barcode of the disclosure. To ensure efficient association between the target and the stochastic barcode, the targets can be crosslinked to the stochastic barcode.

### Cell Lysis

Following the distribution of cells and stochastic barcodes, the cells can be lysed to liberate the target molecules. Cell lysis can be accomplished by any of a variety of means, for example, by chemical or biochemical means, by osmotic shock, or by means of thermal lysis, mechanical lysis, or optical lysis. Cells can be lysed by addition of a cell lysis buffer comprising a detergent (e.g., SDS, Li dodecyl sulfate, Triton X-100, Tween-20, or NP-40), an organic solvent (e.g. methanol or acetone), or digestive enzymes (e.g. proteinase K, pepsin, or trypsin), or any combination thereof. To increase the association of a target and a stochastic barcode, the rate of the diffusion of the target molecules can be altered by for example, reducing the temperature and/or increasing the viscosity of the lysate.

In some embodiments, the sample can be lysed using a filter paper. The filter paper can be soaked with a lysis buffer on top of the filter paper. The filter paper can be applied to the sample with pressure which can facilitate lysis of the sample and hybridization of the targets of the sample to the substrate.

In some embodiments, lysis can be performed by mechanical lysis, heat lysis, optical lysis, and/or chemical lysis. Chemical lysis can include the use of digestive enzymes such as proteinase K, pepsin, and trypsin. Lysis can be performed by the addition of a lysis buffer to the substrate. A lysis buffer can comprise Tris HCl. A lysis buffer can comprise at least about 0.01, 0.05, 0.1, 0.5, or 1 M or more Tris HCl. A lysis buffer can comprise at most about 0.01, 0.05, 0.1, 0.5, or 1 M or more Tris HCL. A lysis buffer can comprise about 0.1 M Tris HCl. The pH of the lysis buffer can be at least about 1, 2, 3, 4, 5, 6, 7, 8, 9, or 10 or more. The pH of the lysis buffer can be at most about 1, 2, 3, 4, 5, 6, 7, 8, 9, or 10 or more. In some embodiments, the pH of the lysis buffer is about 7.5. The lysis buffer can comprise a salt (e.g., LiCl). The concentration of salt in the lysis buffer can be at least about 0.1, 0.5, or 1 M or more. The concentration of salt in the lysis buffer can be at most about 0.1, 0.5, or 1 M or more. In some embodiments, the concentration of salt in the lysis buffer is about 0.5M. The lysis buffer can comprise a detergent (e.g., SDS, Li dodecyl sulfate, triton X, tween, NP-40). The concentration of the detergent in the lysis buffer can be at least about 0.0001%, 0.0005%, 0.001%, 0.005%, 0.01%, 0.05%, 0.1%, 0.5%, 1%, 2%, 3%, 4%, 5%, 6%, or 7% or more. The concentration of the detergent in the lysis buffer can be at most about 0.0001%, 0.0005%, 0.001%, 0.005%, 0.01%, 0.05%, 0.1%, 0.5%, 1%, 2%, 3%, 4%, 5%, 6%, or 7% or more. In some embodiments, the concentration of the detergent in the lysis buffer is about 1% Li dodecyl sulfate. The time used in the method for lysis can be dependent on the amount of detergent used. In some embodiments, the more detergent used, the less time needed for lysis. The lysis buffer can comprise a chelating agent (e.g., EDTA, EGTA). The concentration of a chelating agent in the lysis buffer can be at least about 1, 5, 10, 15, 20, 25, or 30 mM or more. The concentration of a chelating agent in the lysis buffer can be at most about 1, 5, 10, 15, 20, 25, or 30 mM or more. In some embodiments, the concentration of chelating agent in the lysis buffer is about 10 mM. The lysis buffer can comprise a reducing reagent (e.g., beta-mercaptoethanol, DTT). The concentration of the reducing reagent in the lysis buffer can be at least about 1, 5, 10, 15, or 20 mM or more. The concentration of the reducing reagent in the lysis buffer can be at most about 1, 5, 10, 15, or 20 mM or more. In some embodiments, the concentration of reducing reagent in the lysis buffer is about 5 mM. In some embodiments, a lysis buffer can comprise about 0.1M TrisHCl, about pH 7.5, about 0.5M LiCl, about 1% lithium dodecyl sulfate, about 10 mM EDTA, and about 5 mM DTT.

Lysis can be performed at a temperature of about 4, 10, 15, 20, 25, or 30° C. Lysis can be performed for about 1, 5, 10, 15, or 20 or more minutes. A lysed cell can comprise at least about 100000, 200000, 300000, 400000, 500000, 600000, or 700000 or more target nucleic acid molecules. A lysed cell can comprise at most about 100000, 200000, 300000, 400000, 500000, 600000, or 700000 or more target nucleic acid molecules.

### Attachment of Stochastic Barcodes to Target Nucleic Acid Molecules

Following lysis of the cells and release of nucleic acid molecules therefrom, the nucleic acid molecules can randomly associate with the stochastic barcodes of the co-localized solid support. Association can comprise hybridization of a stochastic barcode's target recognition region to a complementary portion of the target nucleic acid molecule (e.g., oligo(dT) of the stochastic barcode can interact with a poly(A) tail of a target). The assay conditions used for hybridization (e.g., buffer pH, ionic strength, temperature, etc.) can be chosen to promote formation of specific, stable hybrids. In some embodiments, the nucleic acid molecules released from the lysed cells can associate with the plurality of probes on the substrate (e.g., hybridize with the probes on the substrate). When the probes comprise oligo(dT), mRNA molecules can hybridize to the probes and be reverse transcribed. The oligo(dT) portion of the oligonucleotide can act as a primer for first strand synthesis of a cDNA molecule. For example, mRNA molecules can hybridize to stochastic barcodes on beads. For example, single-stranded nucleotide fragments can hybridize to the target-binding regions of stochastic barcodes.

Attachment can further comprise ligation of a stochastic barcode's target recognition region and a portion of the target nucleic acid molecule. For example, the target binding region can comprise a nucleic acid sequence that can be capable of specific hybridization to a restriction site overhang (e.g. an EcoRI sticky-end overhang). The assay procedure can further comprise treating the target nucleic acids with a restriction enzyme (e.g. EcoRI) to create a restriction site overhang. The stochastic barcode can then be ligated to any nucleic acid molecule comprising a sequence complementary to the restriction site overhang. A ligase (e.g., T4 DNA ligase) can be used to join the two fragments.

For example, the labeled targets from a plurality of cells (or a plurality of samples) (e.g., target-barcode molecules) can be subsequently pooled, for example, into a tube. The labeled targets can be pooled by, for example, retrieving the stochastic barcodes and/or the beads to which the target-barcode molecules are attached.

The retrieval of solid support-based collections of attached target-barcode molecules can be implemented by use of magnetic beads and an externally-applied magnetic field. Once the target-barcode molecules have been pooled, all further processing can proceed in a single reaction vessel. Further processing can include, for example, reverse transcription reactions, amplification reactions, cleavage reactions, dissociation reactions, and/or nucleic acid extension reactions. Further processing reactions can be performed within the microwells, that is, without first pooling the labeled target nucleic acid molecules from a plurality of cells.

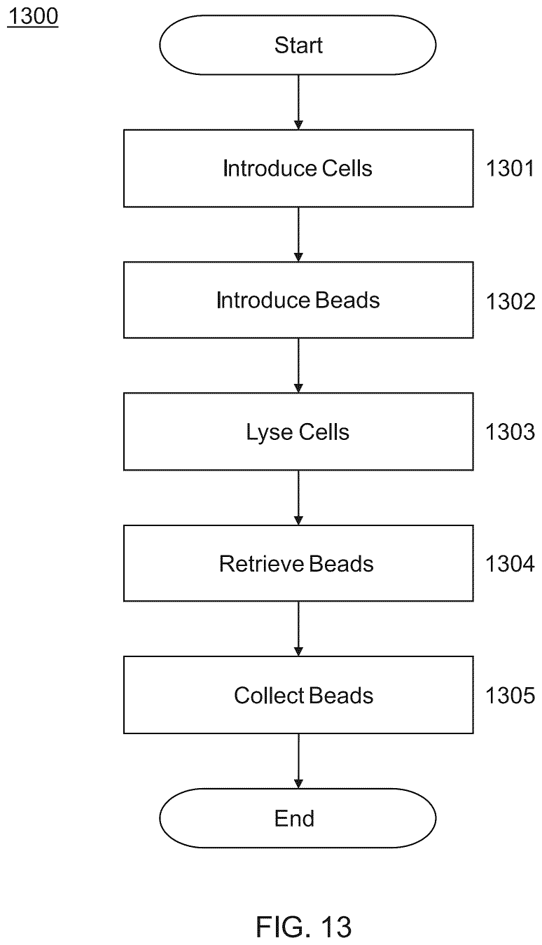

FIG. 13 depicts a non-limiting example of a workflow 1300 that can be performed using a system and multi-microwell-array-flowcell cartridge of the invention. The workflow 1300 describes steps performed after the multi-microwell-array-flowcell cartridge is positioned within the tray and the sample collection vessels are positioned within the drawer. Additional steps of inserting the cartridge into the tray and/or inserting the sample collection vessels or sample collection vessel holder may be required prior to initiation of the workflow 1300 if these components are not already in their respective positions.

Additionally, the workflow 1300 describes steps performed with the retrieval magnet assembly actuator in its inactive position and the drawer actuator in a waste collection position at the beginning of the workflow 1300. One of skill in the art would recognize that additional steps may be required to move the actuators to initiation of the workflow.

The workflow 1300 can begin with a step 1301 in which a plurality of cells is introduced into one or more fluidic lanes of a cartridge while the cartridge is positioned within a system of the invention. In some embodiments, the plurality of cells can be introduced into the cartridge via the inlets (e.g., gaskets). In some embodiments, the plurality of cells can be introduced into the cartridge via a pipette. In some embodiments, the plurality of cells can enter the microwell array via the flowcells of the cartridge. In some embodiments, each microwell in the microwell array can entrap only a single cell of the plurality of cells. In alternative embodiments, the plurality of cells can be introduced into the microwell array prior to positioning the cartridge within the system.

After the cells are introduced into the microwell array, a plurality of barcode-bearing beads can be introduced into the microwell array(s) at a step 1302. In some embodiments, the plurality of beads can be introduced into the cartridge inlets. In some embodiments, the plurality of beads can be introduced into the cartridge via a pipette. In some embodiments, the plurality of beads can enter the microwell array via the flowcell of the cartridge. In alternative embodiments, the plurality of beads can be introduced into the microwell array prior to positioning of the cartridge within the system of the invention. It would be appreciated by one of skill in the art that the order in which the cells are introduced (block 1301) and the beads are introduced (block 1302) occur is not particularly limited. The two steps can occur concurrently or sequentially, and any order is within the scope of the present disclosure. In some embodiments, each microwell in the microwell array can entrap only a single bead of the plurality of beads. In some embodiments, each microwell in the microwell array can entrap a single cell of the plurality of cells and a single bead of the plurality of beads.

After the plurality of beads are introduced into the microwell array, cell lysis can be performed at a step 1303. In some embodiments, the cell lysis can be performed before the plurality of beads are introduced into the microwell array. Cell lysis can be accomplished by any of the variety of means described herein. In some embodiments, step 1303 does not include activating a lysis magnet. In some embodiments, the beads can be dimensioned such that a bead positioned superior to a cell within a microwell can prevent passage of the cell out of the microwell without removal of the bead from the microwell.

In some embodiments, lysis is performed by introducing a lysis buffer. In some embodiments, during cell lysis, the drawer actuator can be used to position the drawer such that the waste collection vessel is positioned under the outlet of the cartridge to receive excess buffer flowing through the flowcells. In some embodiments, the introduction of cells and beads results in cells and/or beads positioned within the flowcells but outside of a microwell. In such embodiments, the cells and/or beads positioned outside of the microwells may be washed away by the lysis buffer into the waste collection vessel.

In certain embodiments, a wash can be performed before, during, or after cell lysis. In some embodiments, a wash fluid can be introduced into the flowcells of the cartridge via the inlets. The wash fluid can flow through the flowcells to remove beads and/or cells within the flowcells but outside of a microwell. The removed beads and/or cells can be deposited in the waste collection vessel aligned with the outlets of the flowcells.

After cell lysis, the barcode-bearing beads can be retrieved at step 1304. In some embodiments, the barcode-bearing beads are retrieved by advancing the retrieval magnet assembly from its inactive position to its active position. As described herein, the retrieval magnet assembly can be advanced from its inactive position to its active position by movement of the retrieval magnet assembly actuator.

When the retrieval magnet assembly is positioned in the active position, the retrieval magnet assembly can attract the barcode-bearing beads positioned within the microwells. In some embodiments, the uniform magnetic force exerted on the barcode-bearing beads by the retrieval magnet assembly can be sufficient to remove the barcode bearing beads from the microwells. When the barcode-bearing beads are removed by the retrieval magnet assembly, the cells may remain within the microwells. In some embodiments, the magnetic force exerted on the barcode-bearing beads by the retrieval magnet assembly can cause the barcode bearing beads to move towards the superior surface of the cartridge. The magnetic force exerted by the retrieval magnet assembly can maintain the beads in a position superior to the microwells. While the beads are maintained in a position superior to the microwells, the beads can be said to be retrieved by the retrieval magnet assembly.

While the beads are maintained in a position superior to the microwells by the retrieval magnet assembly, a wash can be performed. In some embodiments, a wash fluid can be introduced into the flowcells of the cartridge via the inlets. The wash fluid can flow through the flowcells to remove the cells in the microwells. The cells can be deposited in the waste collection vessel aligned with the outlet of the flowcells. Washing the cells from the microwells can allow for later collection of only the beads previously positioned in the microwells. In some embodiments, actuation of the retrieval magnet assembly causes an interlock to engage the drawer such that the drawer is prevented from moving.

After the beads are retrieved by the retrieval magnet assembly, the beads can be collected at step 1305. In some embodiments, the retrieval magnet assembly is transitioned from its active position to its inactive position, releasing the beads from their maintained position superior to the microwells. As described herein, the retrieval magnet assembly can be transitioned from its active position to its inactive position by movement of the retrieval magnet assembly actuator. In some embodiments, upon release, the beads drop or return into the same microwells from which they were removed.

After the beads are released, the sample collection vessels in the sample collection holder can be aligned with the outlets of the cartridge. As described herein, the sample collection vessels in the sample collection holder can be aligned with the outlets by transitioning the actuator to a sample collection position. After alignment, a fluid can be advanced through the flowcells to cause the beads to flow out of the outlets and into the sample collection vessels. To allow collection of the beads, it can be desirable that the retrieval magnet assembly is placed in its inactive position. After the beads are collected, the workflow 1300 concludes. In some embodiments, after collection of the beads, the sample collection vessels can be removed for further processing and/or analysis of the beads.

### Library Preparation

In some instances, methods also include sequence library preparation protocols for further processing the analyte (e.g., DNA, RNA) obtained by the steps provided above to generate a sequence library (e.g., a cDNA library). In certain instances, methods include producing a sequence ready nucleic acid library from the processed sample. In certain versions, the sequence ready nucleic acid library is sequence-able by using a next generation sequencing (NGS) protocol.

In some embodiments, barcoding (e.g., stochastically barcoding) the plurality of targets in the sample further comprises generating an indexed library of the barcoded targets (e.g., stochastically barcoded targets) or barcoded fragments of the targets.

Creation of an indexed library is discussed in, e.g., U.S. Pat. No. 10,676,779 as well as U.S. Patent Application Publication No. 2021/0171940; the disclosures of which are incorporated by reference herein in their entirety. The barcode sequences of different barcodes (e.g., the molecular labels of different stochastic barcodes) can be different from one another. Generating an indexed library of the barcoded targets includes generating a plurality of indexed polynucleotides from the plurality of targets in the sample. For example, for an indexed library of the barcoded targets comprising a first indexed target and a second indexed target, the label region of the first indexed polynucleotide can differ from the label region of the second indexed polynucleotide by, by about, by at least, or by at most, 1, 2, 3, 4, 5, 6, 7, 8, 9, 10, 20, 30, 40, 50, or a number or a range between any two of these values, nucleotides. In some embodiments, generating an indexed library of the barcoded targets includes contacting a plurality of targets, for example mRNA molecules, with a plurality of oligonucleotides including a poly(T) region and a label region; and conducting a first strand synthesis using a reverse transcriptase to produce single-strand labeled cDNA molecules each comprising a cDNA region and a label region, wherein the plurality of targets includes at least two mRNA molecules of different sequences and the plurality of oligonucleotides includes at least two oligonucleotides of different sequences. Generating an indexed library of the barcoded targets can further comprise amplifying the single-strand labeled cDNA molecules to produce double-strand labeled cDNA molecules; and conducting nested PCR on the double-strand labeled cDNA molecules to produce labeled amplicons. In some embodiments, the method can include generating an adaptor-labeled amplicon.

Barcoding (e.g., stochastic barcoding) can include using nucleic acid barcodes or tags to label individual nucleic acid (e.g., DNA or RNA) molecules. In some embodiments, it involves adding DNA barcodes or tags to cDNA molecules as they are generated from mRNA. Nested PCR can be performed to minimize PCR amplification bias. Adaptors can be added for sequencing using, for example, next generation sequencing (NGS). The sequencing results can be used to determine cell labels, molecular labels, and sequences of nucleotide fragments of the one or more copies of the targets.

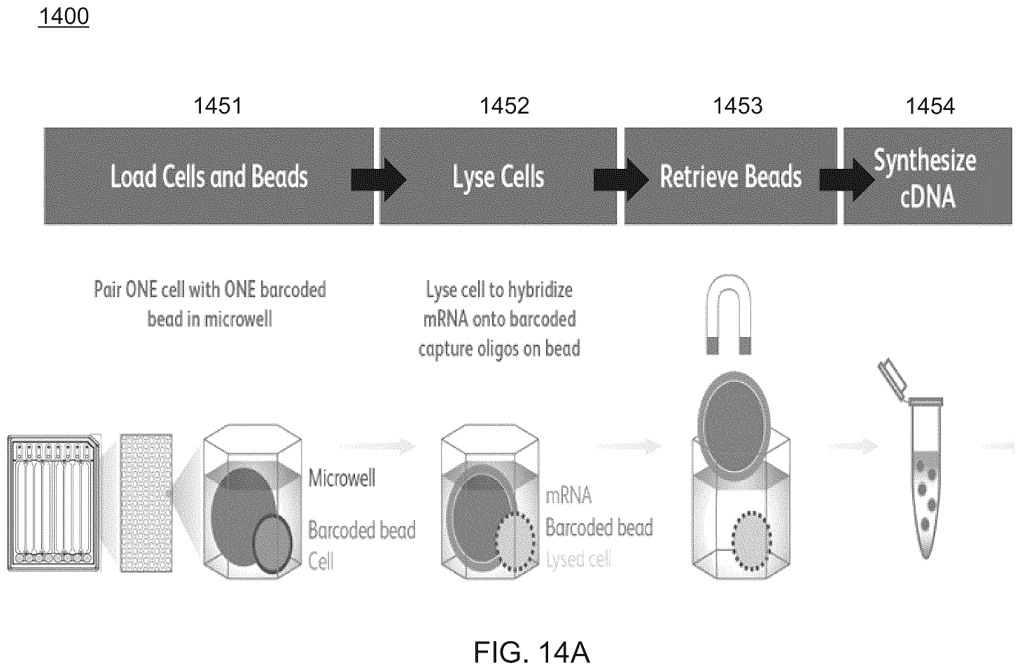

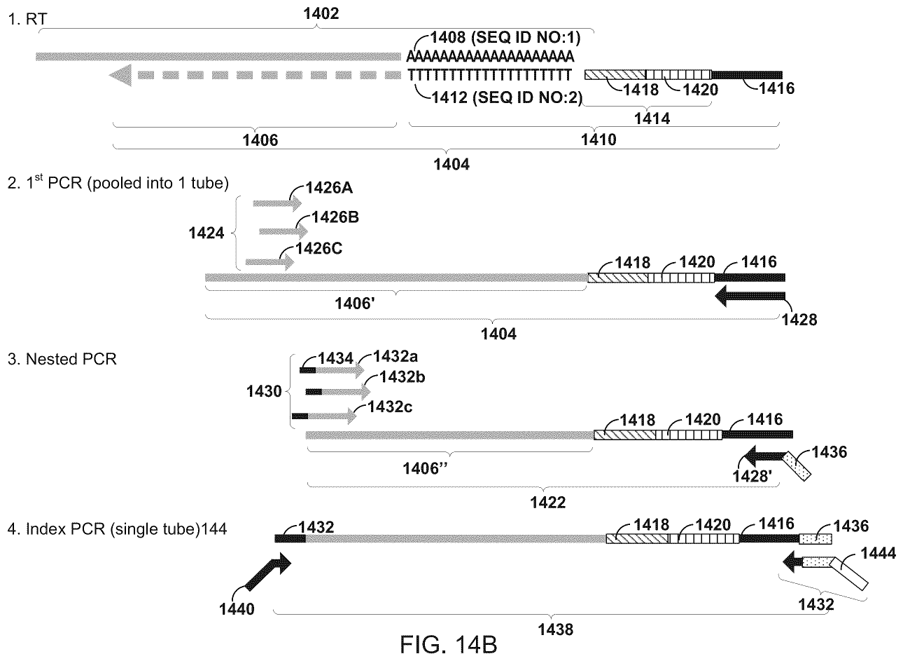

FIG. 14A presents a workflow for practicing a certain embodiment of the subject methods. As shown in step 1451, cells and beads are loaded in a cartridge of the invention. One cell is paired with one barcoded bead in a microwell. In step 1452, the cells are lysed and nucleic acid from the cells is hybridized to the bead. In step 1453, a retrieval magnet assembly is employed to retrieve the beads. In step 1454, the nucleic acid is used to synthesize cDNA.

FIG. 14B is a schematic illustration showing a non-limiting exemplary process of generating an indexed library of the barcoded targets (e.g., stochastically barcoded targets), such as barcoded mRNAs or fragments thereof. In other words, FIG. 14B shows an exemplary cDNA synthesis step 1454 shown in FIG. 14A. As shown in step 1, the reverse transcription process can encode each mRNA molecule with a unique molecular label, a cell label, and a universal PCR site. In particular, RNA molecules 1402 can be reverse transcribed to produce labeled cDNA molecules 1404, including a cDNA region 1406, by hybridization (e.g., stochastic hybridization) of a set of barcodes (e.g., stochastic barcodes) 1410 to the poly(A) tail region 1408 of the RNA molecules 1402. Each of the barcodes 1410 can comprise a target-binding region, for example a poly(dT) region 1412, a label region 1414 (e.g., a barcode sequence or a molecule), and a universal PCR region 1416. In some embodiments, the cell label can include 3 to 20 nucleotides. In some embodiments, the molecular label can include 3 to 20 nucleotides. In some embodiments, each of the plurality of stochastic barcodes further comprises one or more of a universal label and a cell label, wherein universal labels are the same for the plurality of stochastic barcodes on the solid support and cell labels are the same for the plurality of stochastic barcodes on the solid support. In some embodiments, the universal label can include 3 to 20 nucleotides. In some embodiments, the cell label comprises 3 to 20 nucleotides.

In some embodiments, the label region 1414 can include a barcode sequence or a molecular label 1418 and a cell label 1420. In some embodiments, the label region 1414 can include one or more of a universal label, a dimension label, and a cell label. The barcode sequence or molecular label 1418 can be, can be about, can be at least, or can be at most, 1, 2, 3, 4, 5, 6, 7, 8, 9, 10, 20, 30, 40, 50, 60, 70, 80, 90, 100, or a number or a range between any of these values, of nucleotides in length. The cell label 1420 can be, can be about, can be at least, or can be at most, 1, 2, 3, 4, 5, 6, 7, 8, 9, 10, 20, 30, 40, 50, 60, 70, 80, 90, 100, or a number or a range between any of these values, of nucleotides in length. The universal label can be, can be about, can be at least, or can be at most, 1, 2, 3, 4, 5, 6, 7, 8, 9, 10, 20, 30, 40, 50, 60, 70, 80, 90, 100, or a number or a range between any of these values, of nucleotides in length. Universal labels can be the same for the plurality of stochastic barcodes on the solid support and cell labels are the same for the plurality of stochastic barcodes on the solid support. The dimension label can be, can be about, can be at least, or can be at most 1, 2, 3, 4, 5, 6, 7, 8, 9, 10, 20, 30, 40, 50, 60, 70, 80, 90, 100, or a number or a range between any of these values, of nucleotides in length.

In some embodiments, the label region 1414 can comprise, comprise about, comprise at least, or comprise at most, 1, 2, 3, 4, 5, 6, 7, 8, 9, 10, 20, 30, 40, 50, 60, 70, 80, 90, 100, 200, 300, 400, 500, 600, 700, 800, 900, 1000, or a number or a range between any of these values, different labels, such as a barcode sequence or a molecular label 1418 and a cell label 1420. Each label can be, can be about, can be at least, or can be at most 1, 2, 3, 4, 5, 6, 7, 8, 9, 10, 20, 30, 40, 50, 60, 70, 80, 90, 100, or a number or a range between any of these values, of nucleotides in length. A set of barcodes or stochastic barcodes 1410 can contain, contain about, contain at least, or can be at most, 10, 20, 40, 50, 70, 80, 90, 102, 103, 104, 105, 106, 107, 108, 109, 1010, 1011, 1012, 1013, 1014, 1015, 1020, or a number or a range between any of these values, barcodes or stochastic barcodes 1410. And the set of barcodes or stochastic barcodes 1410 can, for example, each contain a unique label region 1414. The labeled cDNA molecules 1404 can be purified to remove excess barcodes or stochastic barcodes 1410. Purification can comprise Ampure bead purification.

As shown in step 2, products from the reverse transcription process in step 1 can be pooled into 1 tube and PCR amplified with a 1^{st} PCR primer pool and a 1^{st} universal PCR primer. Pooling is possible because of the unique label region 1414. In particular, the labeled cDNA molecules 1404 can be amplified to produce nested PCR labeled amplicons 1422. Amplification can comprise multiplex PCR amplification. Amplification can comprise a multiplex PCR amplification with 96 multiplex primers in a single reaction volume. In some embodiments, multiplex PCR amplification can utilize, utilize about, utilize at least, or utilize at most, 10, 20, 40, 50, 70, 80, 90, 102, 103, 104, 105, 106, 107, 108, 109, 1010, 1011, 1012, 1013, 1014, 1015, 1020, or a number or a range between any of these values, multiplex primers in a single reaction volume. Amplification can comprise using a 1^{st} PCR primer pool 1424 comprising custom primers 1426A-C targeting specific genes and a universal primer 1428. The custom primers 1426A-C can hybridize to a region within the cDNA portion 1406′ of the labeled cDNA molecule 1404. The universal primer 1428 can hybridize to the universal PCR region 1416 of the labeled cDNA molecule 304.

As shown in step 3 of FIG. 14B, products from PCR amplification in step 2 can be amplified with a nested PCR primers pool and a 2^{nd} universal PCR primer. Nested PCR can minimize PCR amplification bias. In particular, the nested PCR labeled amplicons 1422 can be further amplified by nested PCR. The nested PCR can comprise multiplex PCR with nested PCR primers pool 1430 of nested PCR primers 1432a-c and a 2^{nd} universal PCR primer 1428′ in a single reaction volume. The nested PCR primer pool can contain, contain about, contain at least, or contain at most, 1, 2, 3, 4, 5, 6, 7, 8, 9, 10, 20, 30, 40, 50, 60, 70, 80, 90, 100, 200, 300, 400, 500, 600, 700, 800, 900, 1000, or a number or a range between any of these values, different nested PCR primers 1430. The nested PCR primers 1432 can contain an adaptor 1434 and hybridize to a region within the cDNA portion 1406″ of the labeled amplicon 1422. The universal primer 1428′ can contain an adaptor 1436 and hybridize to the universal PCR region 1416 of the labeled amplicon 1422. Thus, step 3 produces adaptor-labeled amplicon 1438. In some embodiments, nested PCR primers 1432 and the 2^{nd} universal PCR primer 1428′ may not contain the adaptors 1434 and 1436. The adaptors 1434 and 1436 can instead be ligated to the products of nested PCR to produce adaptor-labeled amplicon 1438.

As shown in step 4, PCR products from step 3 can be PCR amplified for sequencing using library amplification primers. In particular, the adaptors 1434 and 1436 can be used to conduct one or more additional assays on the adaptor-labeled amplicon 1438. The adaptors 1434 and 1436 can be hybridized to primers 1440 and 1442. The one or more primers 1440 and 1442 can be PCR amplification primers. The one or more primers 1440 and 1442 can be sequencing primers. The one or more adaptors 1434 and 1436 can be used for further amplification of the adaptor-labeled amplicons 1438. The one or more adaptors 1434 and 1436 can be used for sequencing the adaptor-labeled amplicon 1438. The primer 1442 can contain a plate index 1444 so that amplicons generated using the same set of barcodes or stochastic barcodes 1410 can be sequenced in one sequencing reaction using next generation sequencing (NGS).

Additional details regarding library preparation workflows using systems of embodiments of the invention, e.g., as described above, may be found in U.S. Pat. Nos. 9,598,736; 10,002,316; 10,527,171; 10,634,691; 10,676,779; 11,061,043; and 11,365,409; as well as U.S. Patent Application Publication Nos. 2016/0340720; 2018/0276332; 2019/0338357; 2020/0124601; 2020/0157600; 2020/0255888; 2020/0299672; 2020/263169; 2020/040379; and 2021/0171940; the disclosures of which are incorporated by reference herein in their entirety.

### Samples

As discussed above, methods include processing a sample. A sample can comprise one or more cells, or nucleic acids from one or more cells. A sample can be a single cell or nucleic acids from a single cell. A sample for use in the method of the disclosure can comprise one or more cells. In some embodiments, the plurality of cells can include one or more cell types. At least one of the one or more cell types can be brain cell, heart cell, cancer cell, circulating tumor cell, organ cell, epithelial cell, metastatic cell, benign cell, primary cell, circulatory cell, or any combination thereof. In some embodiments, the cells are cancer cells excised from a cancerous tissue, for example, breast cancer, lung cancer, colon cancer, prostate cancer, ovarian cancer, pancreatic cancer, brain cancer, melanoma and non-melanoma skin cancers, and the like. In some embodiments, the cells are derived from a cancer but collected from a bodily fluid (e.g., circulating tumor cells). Non-limiting examples of cancers can include, adenoma, adenocarcinoma, squamous cell carcinoma, basal cell carcinoma, small cell carcinoma, large cell undifferentiated carcinoma, chondrosarcoma, and fibrosarcoma. The sample can include a tissue, a cell monolayer, fixed cells, a tissue section, or any combination thereof. The sample can include a biological sample, a clinical sample, an environmental sample, a biological fluid, a tissue, or a cell from a subject. The sample can be obtained from a human, a mammal, a dog, a rat, a mouse, a fish, a fly, a worm, a plant, a fungus, a bacterium, a virus, a vertebrate, or an invertebrate.

In some embodiments, the cells are cells that have been infected with virus and contain viral oligonucleotides. In some embodiments, the viral infection can be caused by a virus such as single-stranded (+strand or “sense”) DNA viruses (e.g., parvoviruses), or double-stranded RNA viruses (e.g., reoviruses). In some embodiments, the cells are bacteria. These can include either gram-positive or gram-negative bacteria. In some embodiments, the cells are fungi. In some embodiments, the cells are protozoans or other parasites.

As used herein, the term “cell” can refer to one or more cells. In some embodiments, the cells are normal cells, for example, human cells in different stages of development, or human cells from different organs or tissue types. In some embodiments, the cells are non-human cells, for example, other types of mammalian cells (e.g., mouse, rat, pig, dog, cow, and horse). In some embodiments, the cells are other types of animal or plant cells. In other embodiments, the cells can be any prokaryotic or eukaryotic cells.

In some embodiments, the cells are sorted prior to associating a cell with a bead. For example, the cells can be sorted by fluorescence-activated cell sorting or magnetic-activated cell sorting, or more generally by flow cytometry. The cells can be filtered by size. In some embodiments, a retentate contains the cells to be associated with the bead. In some embodiments, the flow through contains the cells to be associated with the bead. A sample can refer to a plurality of cells. The sample can refer to a monolayer of cells. The sample can refer to a thin section (e.g., tissue thin section). The sample can refer to a solid or semi-solid collection of cells that can be place in one dimension on an array.

### Kits

As discussed above, aspects of the invention additionally include kits. Kits of the invention include the multi-microwell-array-flowcell cartridge of the invention (e.g., described above). In some cases, kits include a plurality of multi-microwell-array-flowcell cartridges. In some embodiments, kits include a sample collection vessel holder configured to receive a plurality of sample collection vessels. The sample collection vessel holder, according to some embodiments, includes a counterweight configured to maintain the sample collection vessel holder in an upright position. In some embodiments, kits include a plurality of sample collection vessels. Kits may also include one or more waste collection vessels.

In some cases, components of the subject kits are provided in one or more sealable containers. In some cases, the one or more sealable containers are resealable containers, i.e., they may be opened and closed. Any convenient container may be employed, such as a pouch, bag, box, etc. In certain embodiments, a desiccant is provided in the one or more containers, i.e., to control moisture content within said containers. Exemplary desiccants include silica gel and the like. In some cases, the multi-microwell-array-flowcell cartridge of the invention may be used or partially used (e.g., by performing methods of the invention with respect to one or more fluidic lanes of the cartridge), and placed in the sealable container for storage. A partially used multi-microwell-array-flowcell cartridge may be removed from the sealable container and used to prepare one or more additional samples, after which the cartridge may be returned to the container, if desired. The sealable containers may be configured to maintain the stability of a partially used cartridge such that it may be operable for subsequent use at one or more time points following the initial use, such as 1 min or more after the initial use, such as 5 mins or more after the initial use, such as 30 mins or more after the initial use, such as 1 hr or more after the initial use, such as 6 hrs or more after the initial use, such as 1 day or more after the initial use, such as 1 week or more after the initial use, such as 2 weeks after the initial use, such as 1 month after the initial use, such as 3 months after the initial use and including 6 months or more after the initial use.

In some cases, kits include barcoded beads, such as where the barcoded beads comprise nucleic acid barcodes comprising a universal primer binding domain, a cell label domain and a target capture domain. In some such cases, the target capture domain is an oligo dT sequence. In certain versions, the nucleic acid barcode further comprises a unique molecular index (UMI). In some embodiments, the kits comprise stochastic barcodes that may not be attached to a bead. The kit can further comprise reagents, e.g. lysis buffers (such as those described above), rinse/wash buffers, hybridization buffers, and reducing agents (e.g., dithiothreitol). In some embodiments, the kit further comprise reagents (e.g., enzymes, primers, dNTPs, NTPs, RNAse inhibitors, or buffers) for performing nucleic acid extension reactions, for example, reverse transcription reactions and primer extension reactions. In some embodiments, the kit further comprises reagents (e.g., enzymes, universal primers, sequencing primers, target-specific primers, or buffers) for performing amplification reactions to prepare sequencing libraries. In some embodiments, the kit can comprise a ligase, a transposase, a reverse transcriptase, a DNA polymerase, an RNase, an exonuclease, or any combination thereof. In some embodiments, the kit comprises reagents for homopolymer tailing of molecules (e.g., a terminal transferase enzyme, and dNTPs). The kit can comprise reagents for, for example, any enzymatic cleavage of the disclosure (e.g., Exol nuclease, restriction enzyme). In some embodiments, the kit can comprise reagents for adaptor ligation (e.g., ligase enzyme, reducing reagent). In some embodiments, the kit can comprise reagents for library preparation (e.g., addition of sequencing library/flow cell primers) which can include, sequencing/flow cell primers, enzymes for attaching the primers, dNTPs, etc.).

Components of the kits may be present in separate containers, or multiple components may be present in a single container. For example, the template switch oligonucleotide and the template switching polymerase may be provided in the same tube, or may be provided in different tubes. In certain embodiments, it may be convenient to provide the components in a lyophilized form, so that they are ready to use and can be stored conveniently at room temperature.

In addition to the above components, the subject kits may further include (in certain embodiments) instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, and the like.

Yet another form of these instructions is a computer readable medium, e.g., diskette, compact disk (CD), portable flash drive, and the like, on which the information has been recorded. Yet another form of these instructions that may be present is a website address which may be used via the internet to access the information at a removed site.

Additional details regarding aspects of the invention may be found in U.S. Pat. Nos. 9,598,736; 10,002,316; 10,527,171; 10,634,691; 10,676,779; 11,061,043; and 11,365,409; as well as U.S. Patent Application Publication Nos. 2016/0340720; 2018/0276332; 2019/0338357; 2020/0124601; 2020/0157600; 2020/0255888; 2020/0299672; 2020/263169; 2020/040379; and 2021/0171940; the disclosures of which are incorporated by reference herein in their entirety.

### Utility

Systems, cartridges, methods and kits of the invention may be employed where it is desirable to prepare a biological sample, e.g., for further analysis. For example, the invention may be employed to prepare the biological sample for use in diagnostic, monitoring, and research purposes. In some cases, the systems, cartridges, methods and kits of the invention may be employed to increase throughput of sample preparation and analysis, such as by 2× or more, 3× or more, 4× or more, 5× or more, 6× or more, 7× or more and including 8× or more. In some cases, the invention may be employed where it is desirable to prepare and analyze multiple samples simultaneously without experiencing a batch effect. In some cases, systems, cartridges, methods and kits of the invention may be used where there is a need for simplifying and economizing mechanisms for sample preparation.

The following examples are offered by way of illustration and not by way of limitation.

### EXPERIMENTAL

### Example 1

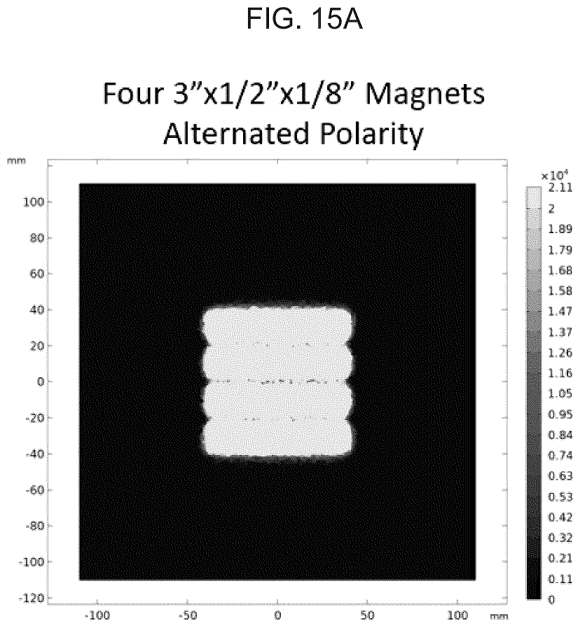

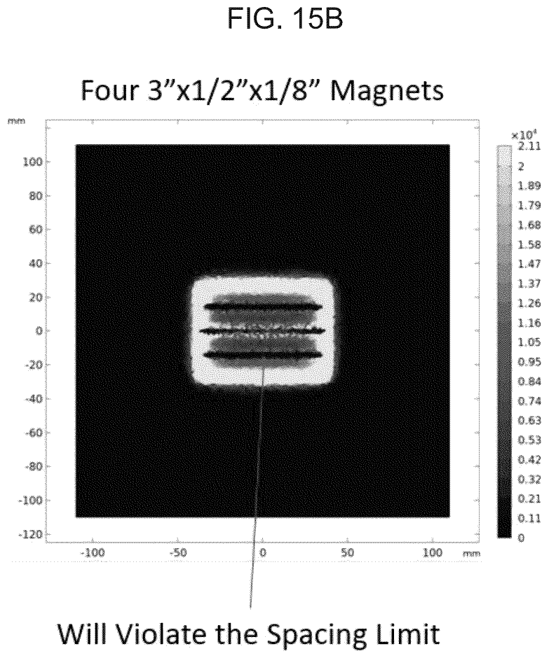

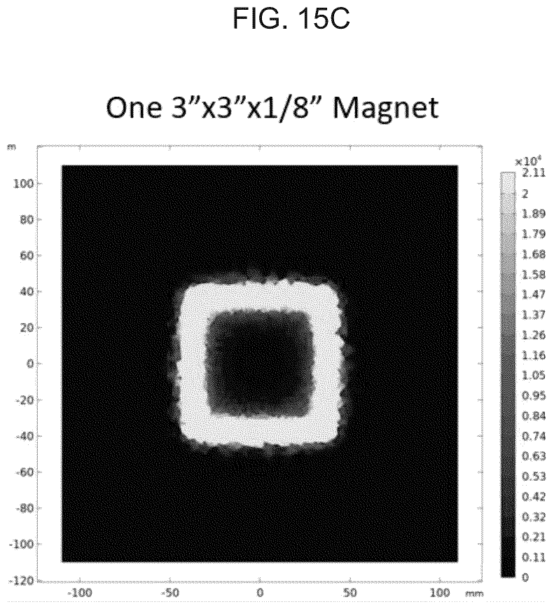

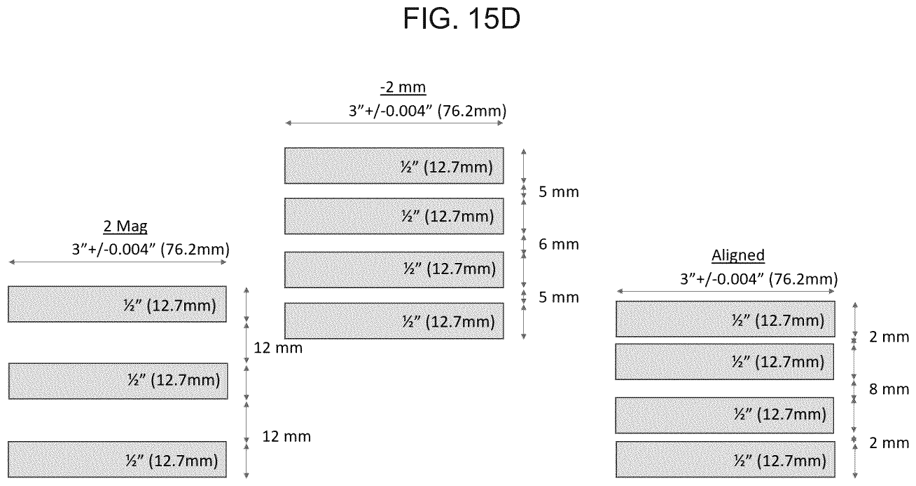

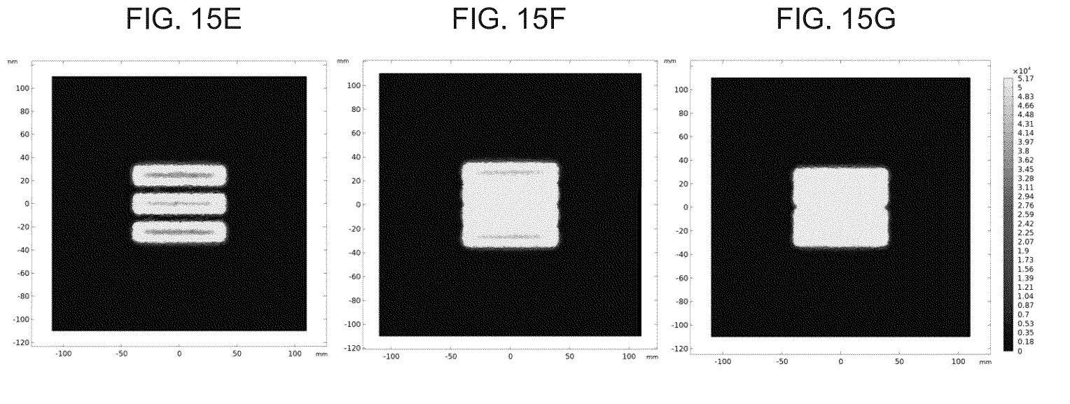

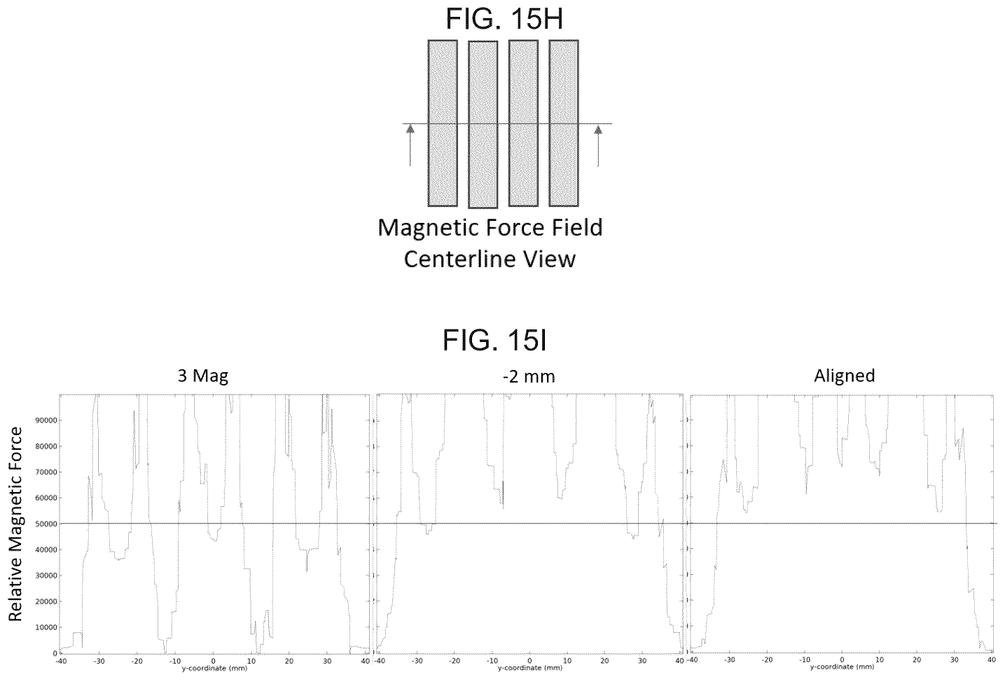

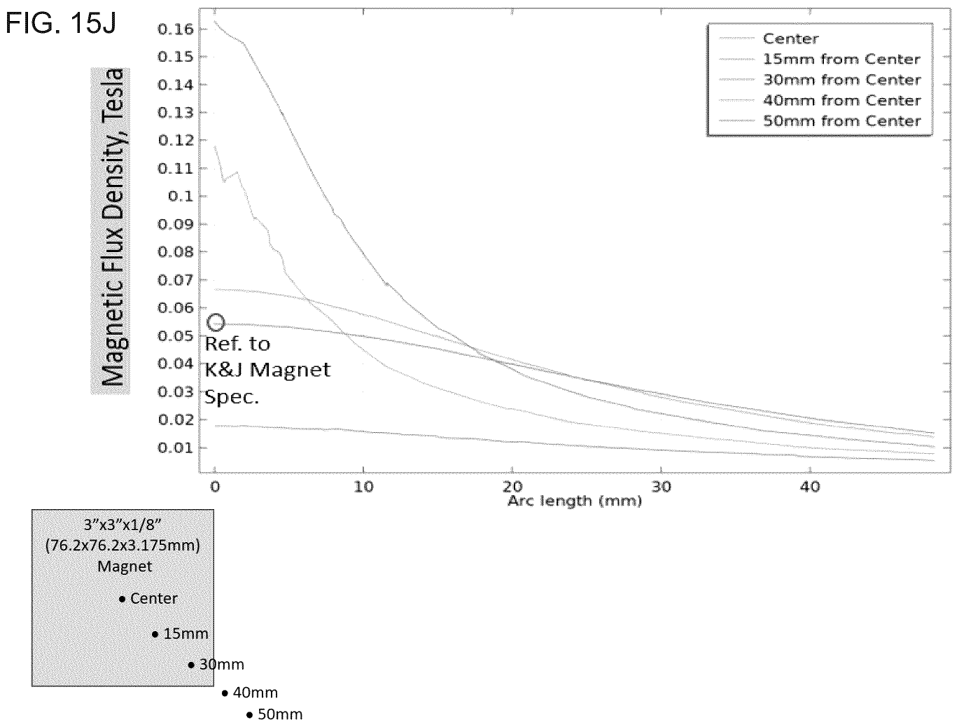

A simulation was carried out in COMSOL Multiphysics® demonstrating the magnetic force resulting from different arrangements of magnets in a retrieval magnet assembly. A simulation of the magnetic force for four 3″×½″×⅛″ magnets having alternating polarity is shown in FIG. 15A. A simulation of the magnetic force for four 3″×½″×⅛″ magnets not having alternating polarity is shown in FIG. 15B. A simulation for one 3″×3″×⅛″ magnet is shown in FIG. 15C. As shown in FIG. 15A-15C, magnets having alternating polarity exhibit more uniform force.

A simulation exploring the effect of distance between adjacent magnets on magnetic force was also carried out. FIG. 15D presents different magnet arrangements that were simulated. FIG. 15E-15G present the results of the simulated arrangements. A significant batch effect was observed in the arrangement shown in FIG. 15E.

A parametric comparison was carried out with respect to the different arrangements shown in FIG. 15D. As shown in FIG. 15I, the “−2 mm” and “aligned” magnet arrangements have similar magnetic force field coverage across the centerline. The centerline is shown in FIG. 15H. As shown in FIG. 15J, the magnetic force is smallest along the centerline.

### Example 2

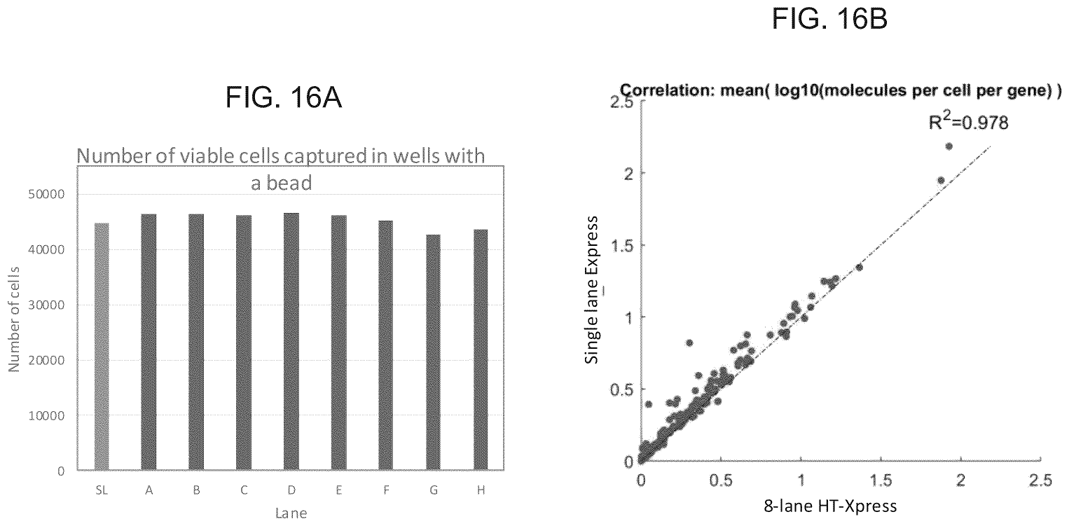

The number of cells captured per lane of an 8-lane Cartridge was measured. >40K cells per lane or >320K viable cells were captured cross 8-lanes (FIG. 16A). An up to 80% capture rate was observed. Multiplet rate comparable to single lane Express/Less than 0.5% cell label collision was enabled by higher diversity beads. No batch effect was observed. High gene-expression correlation was observed with multiple and lanes and with single lane Rhapsody (FIG. 16B).

### Example 3

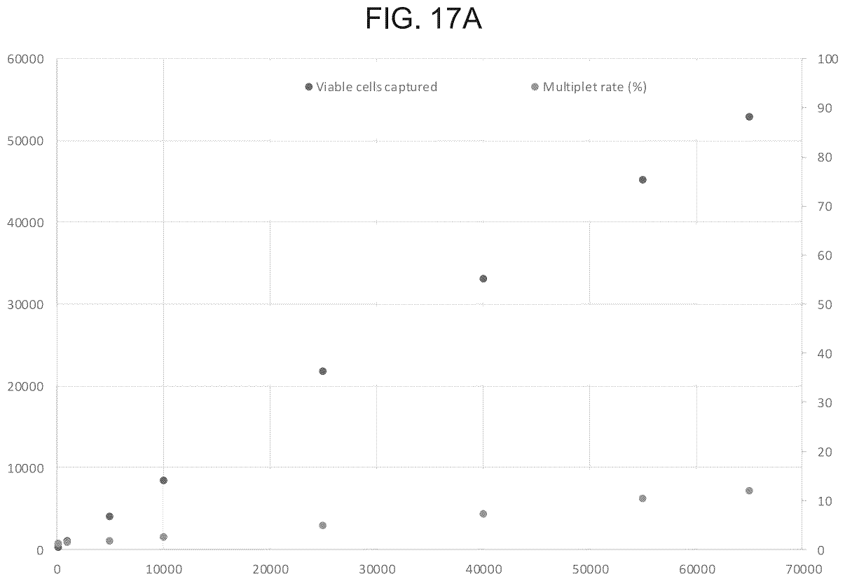

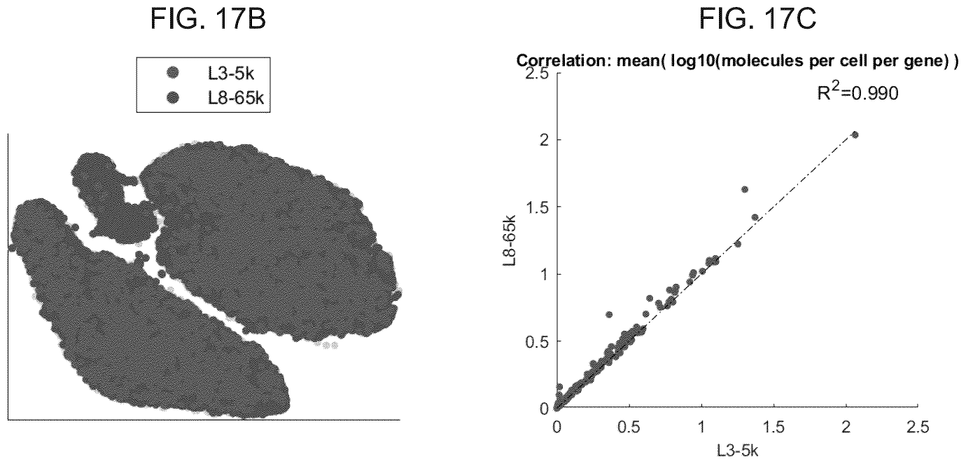

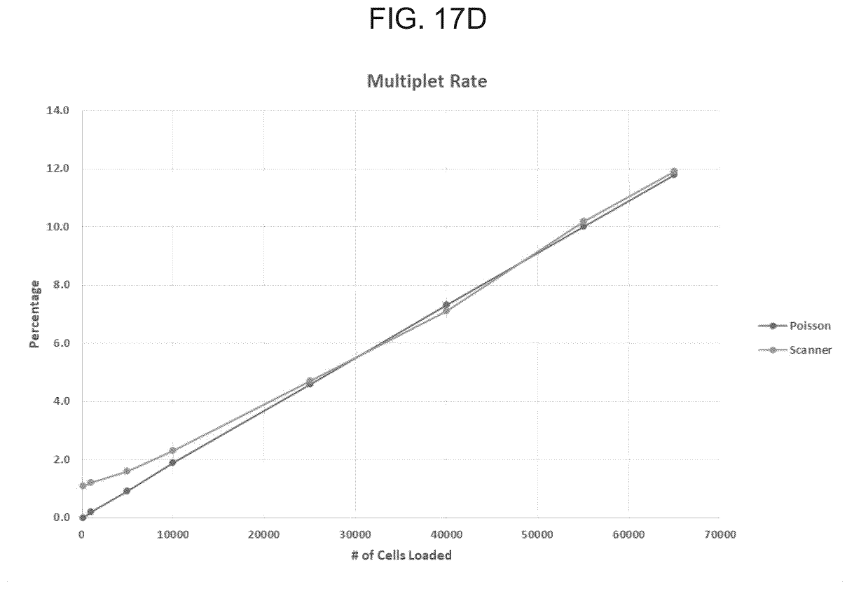

Flexible cell throughput per lane with BD Rhapsody™ HT-Xpress was demonstrated, and the results are depicted in FIG. 17A-17D. All cell loadings were performed on a single 8 lane cartridge and 100, 1,000, 5,000, 10,000, 25,000, 40,000, 55,000, and 65,000 cells were all loaded at the same time respectively from lane 1 through lane 8. The findings correlate to the theoretical Poisson Multiplet load for loadings of 10,000 cells or more and meet metrics for cell capture efficiency and multiplet rate.

### Example 4

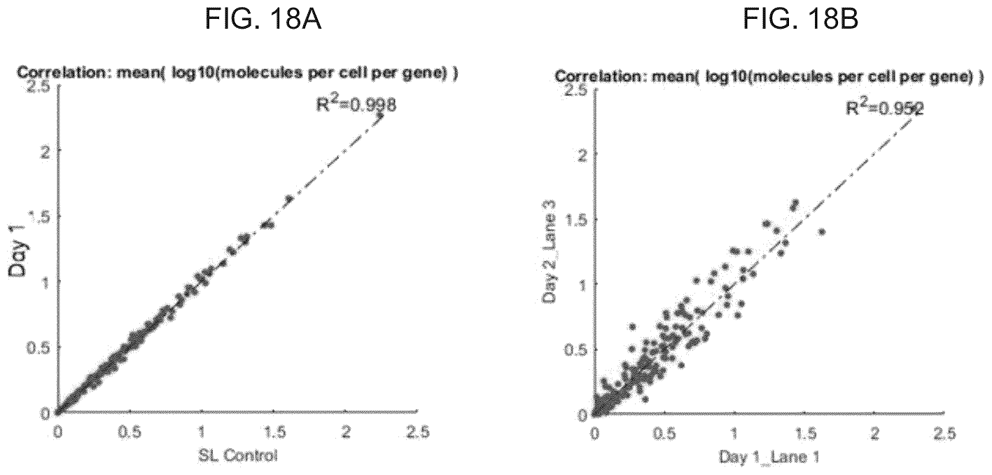

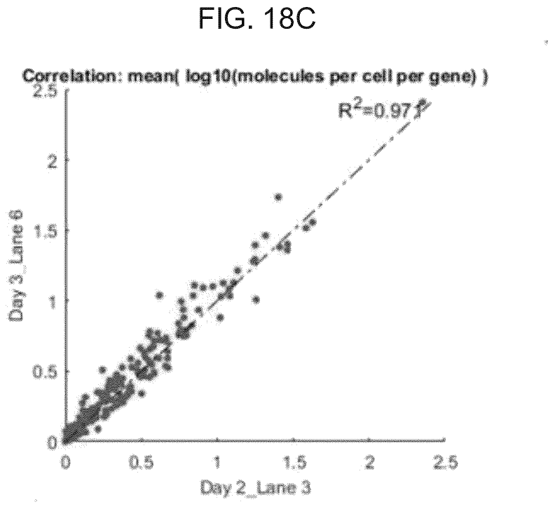

A single 8-lane cartridge was partially used across multiple days. Some of the 1-8 lanes were used at a time, and remaining lanes were used at a different time for the same or different assay. On day 1, lanes 1 and 2 were used. On day 2, lanes 3, 4, and 5 were used. On day 3, lanes 6, 7 and 8 were used. The results are shown in FIG. 18A-18C.

### Example 5

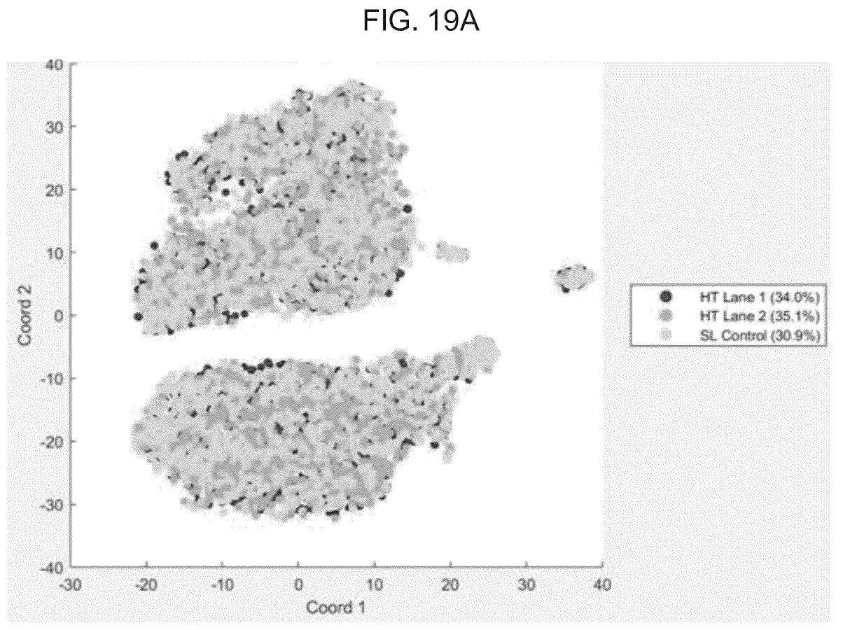

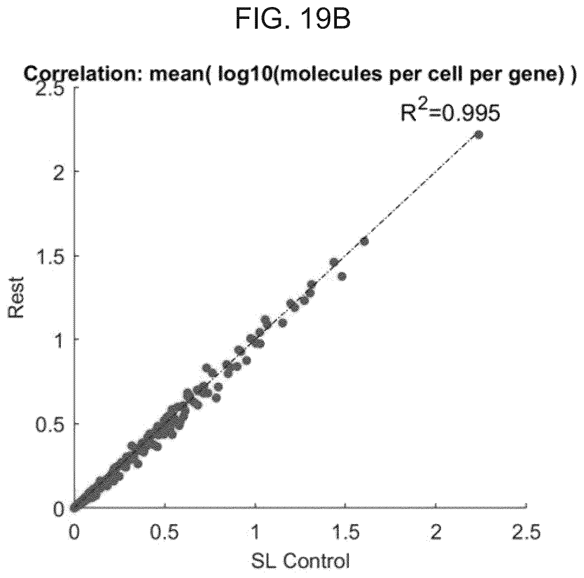

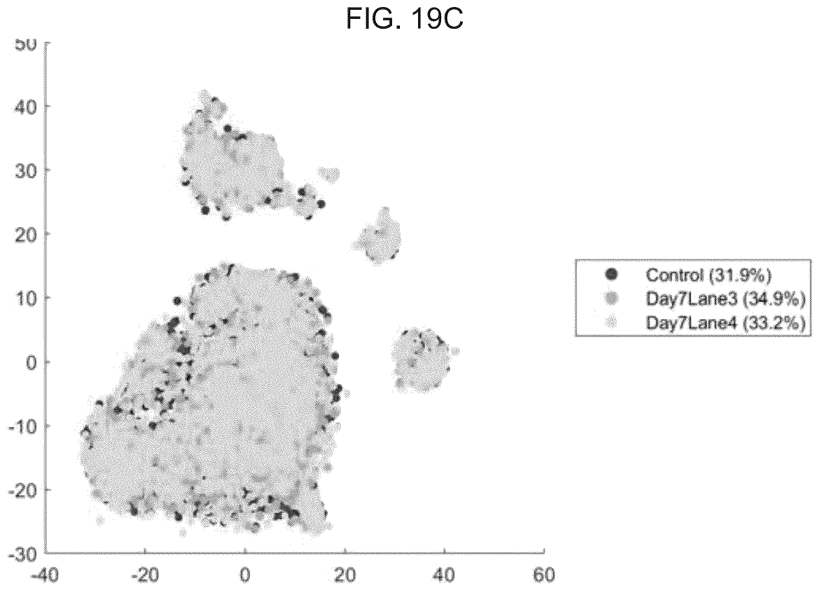

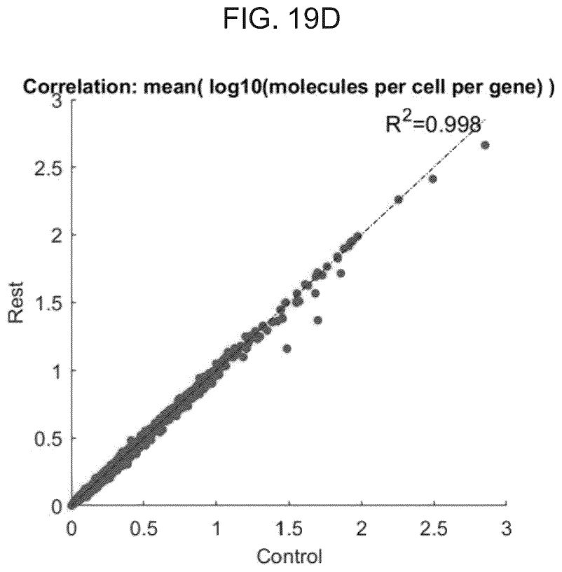

The stability of partially used cartridges for up to 4 months was studied. FIG. 19A-19B show a targeted+SMK assay in lanes 1 and 2 of an 8-lane cartridge using 1:1 Jurkat/Ramos Cells on day 1. FIG. 19C-19D shows WTA+Abseq assay in lanes 3 and 4 of an 8-lane cartridge using PBMCs. Day 1 and 7 on different lanes show tight correlation with single lane control.

### Example 6

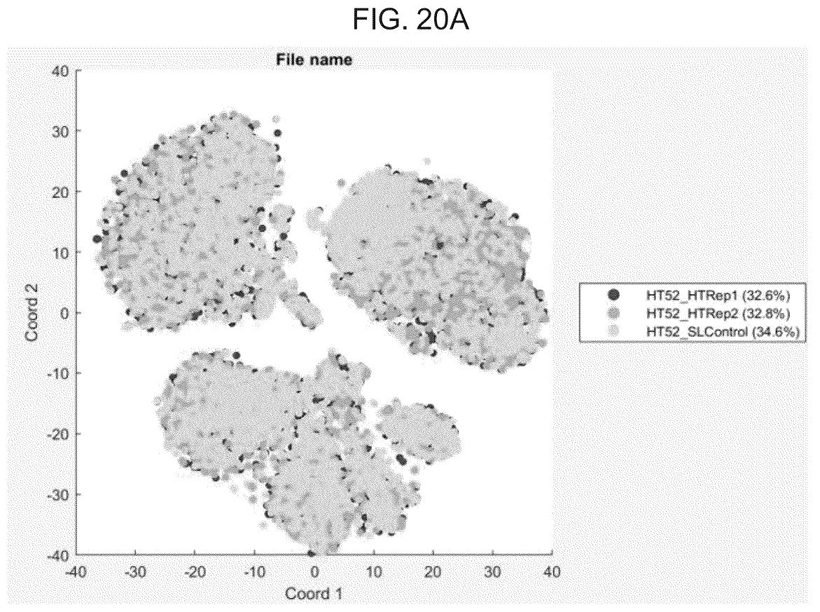

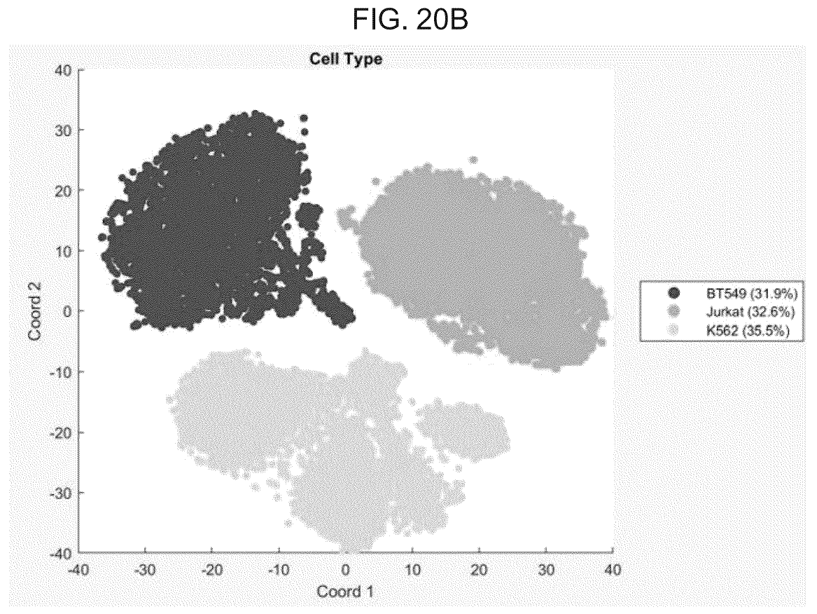

1:1:1 mix of cells of varying sizes were run on an 8-lane cartridge. The cells of different sizes were captured with similar efficiency (80% capture rate at 20K load). No batch effect was observed on samples captured with the single lane and 8-lane cartridge. t-SNE analysis show Jurkat/BT549/K562 cell clusters (FIG. 20A-20B).

### Example 7

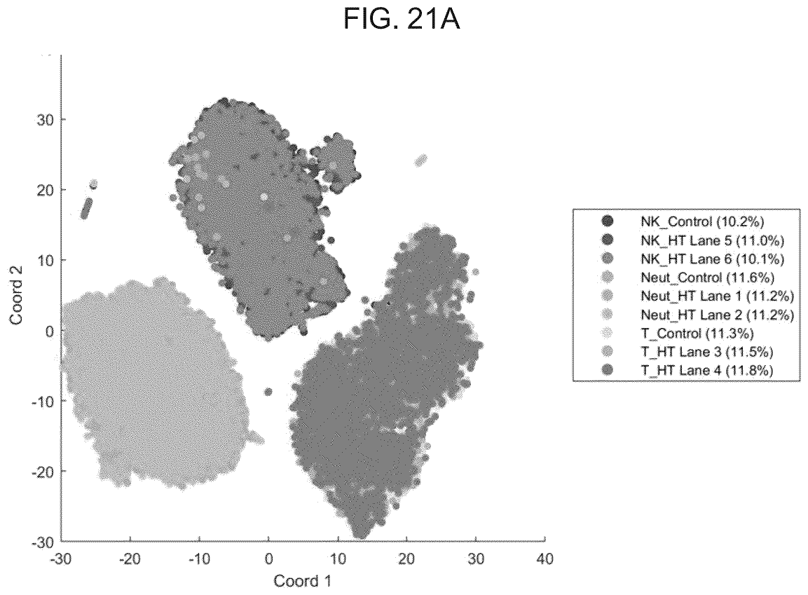

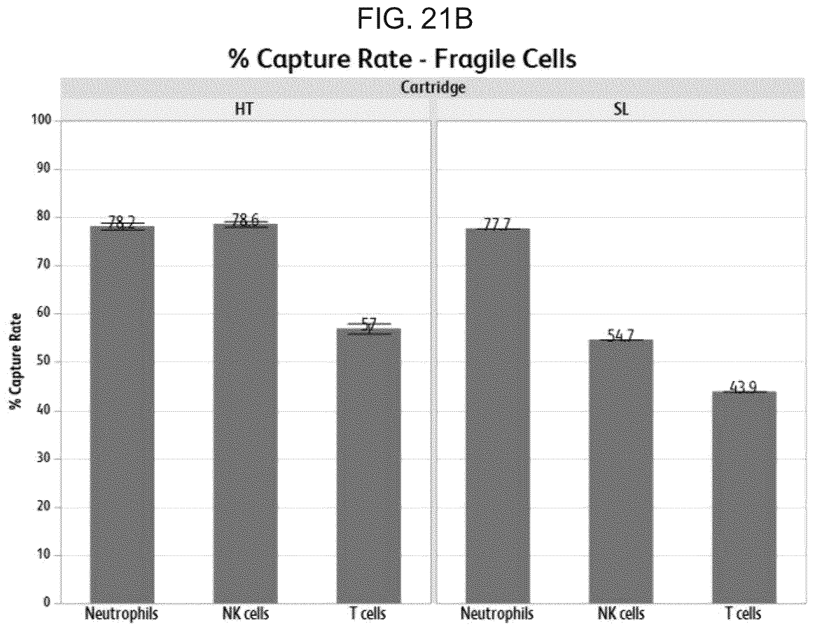

The ability of an 8-lane cartridge to capture enriched neutrophils and sorted NK and T-cells as compared to a single lane cartridge was assessed. No batch effect was observed on samples captured with the single lane and 8-lane cartridge. t-SNE analysis show T cells/NK cells/Neutrophils cell clusters (FIG. 21A). FIG. 21B shows the capture rate with which the different cells were captured.

### Example 8

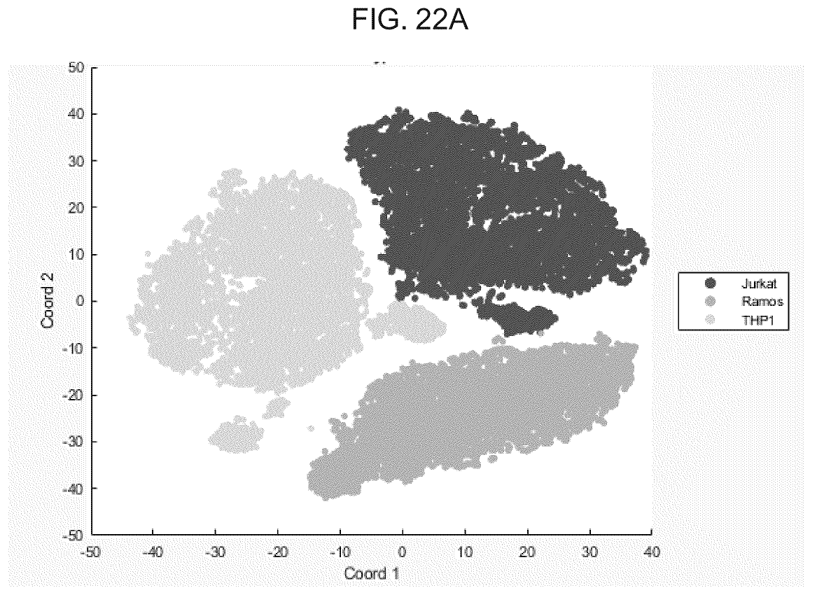

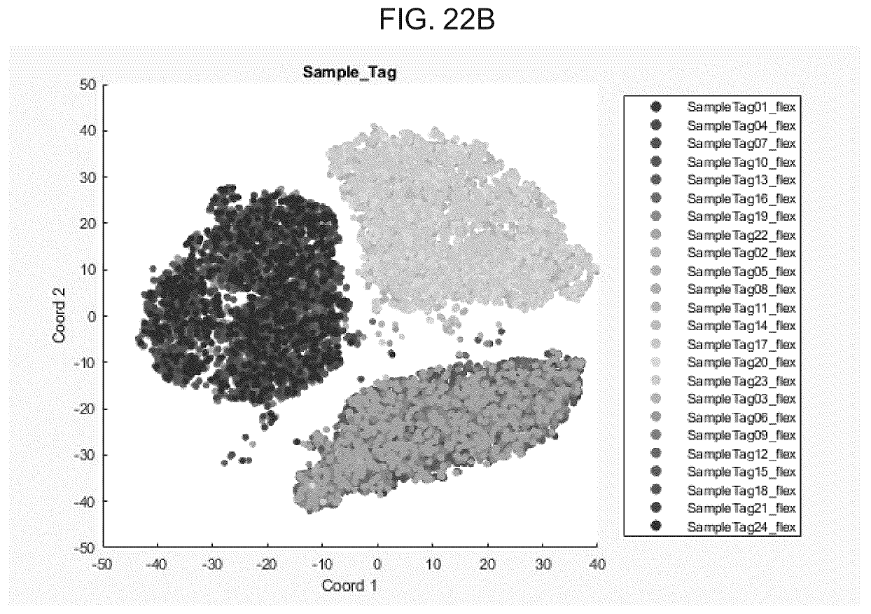

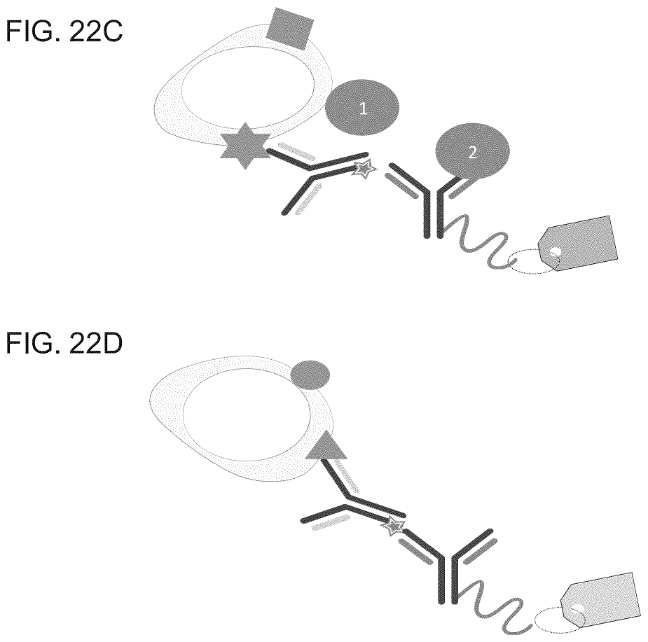

24 sample tubes of Jurkat/Ramos/THP1 cells were stained with 24 Flex SMK tags and mixed prior to Rhapsody workflow. A targeted+SMK experiment was conducted demonstrating >95% sensitivity and specificity for 24 sample tags (FIG. 22A-22B). Two-step staining in which Anti-Beta2-microglobulin is the primary antibody was employed (FIG. 22C-22D).

### Example 9

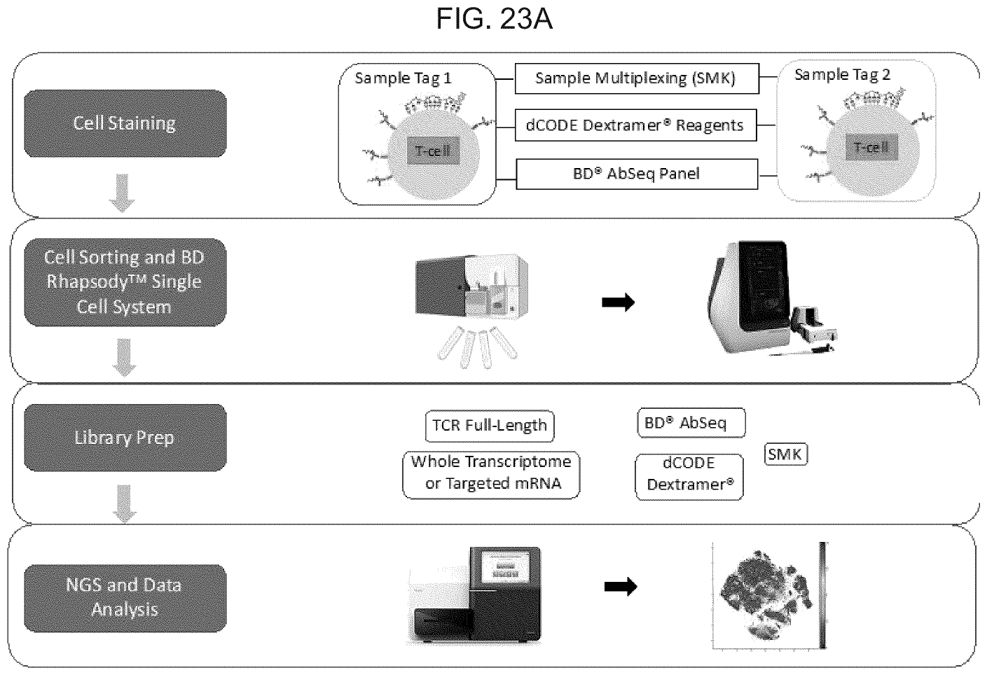

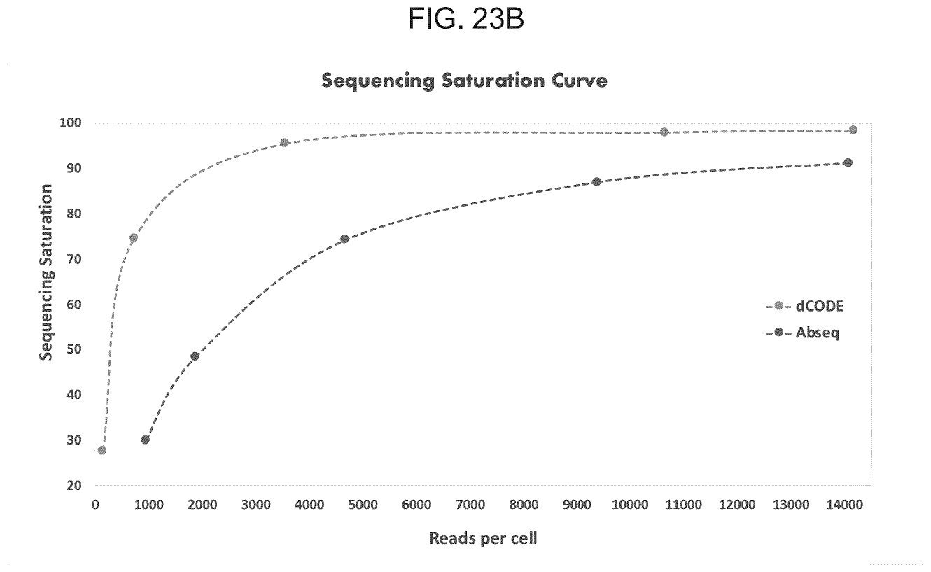

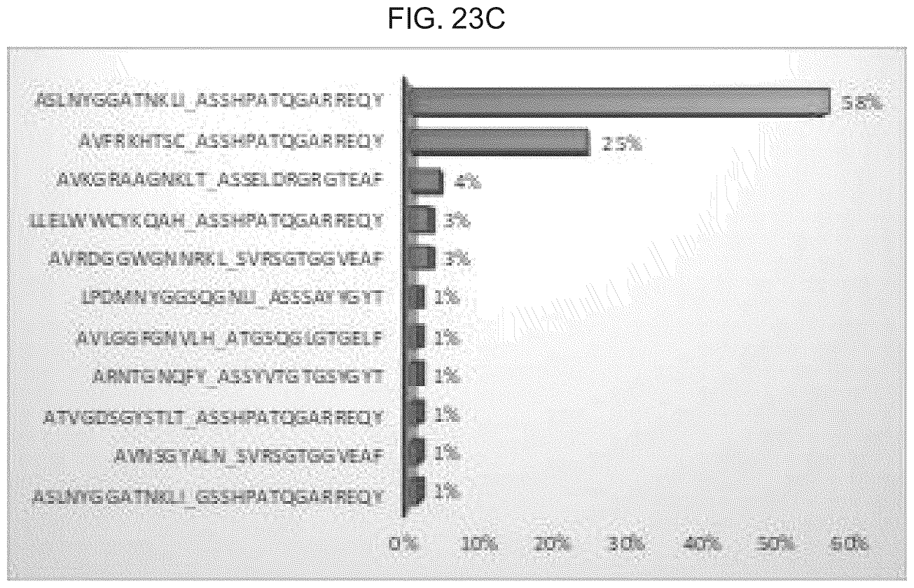

FIG. 23A presents a workflow for characterizing a full length T-cell receptor sequence, along with transcriptome and surface proteins for antigen-specific T-cells. Good resolution between Abseq (CITE-seq) and dCODE reads is shown in FIG. 23B. As shown in FIG. 23C, TCR alpha/beta chain clonotypes were found in a cluster of cells that had high detection of a specific antigen CMV3 dCODE.

### Example 10

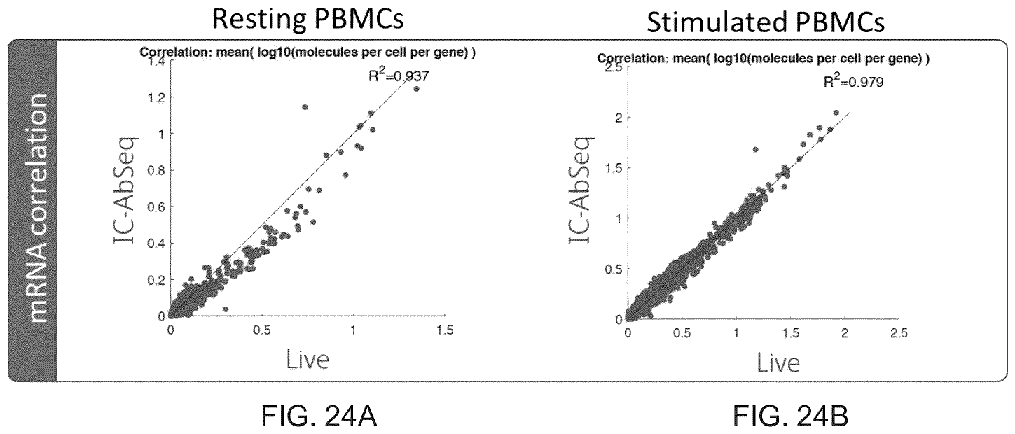

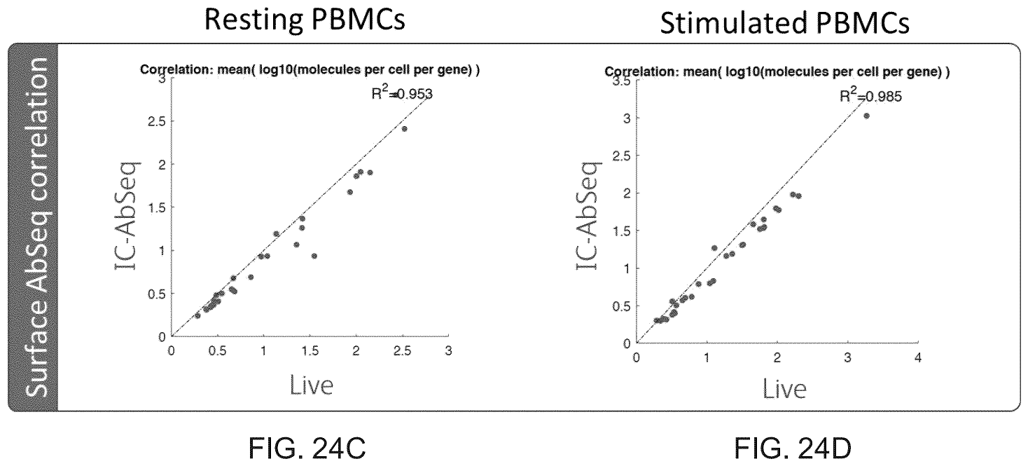

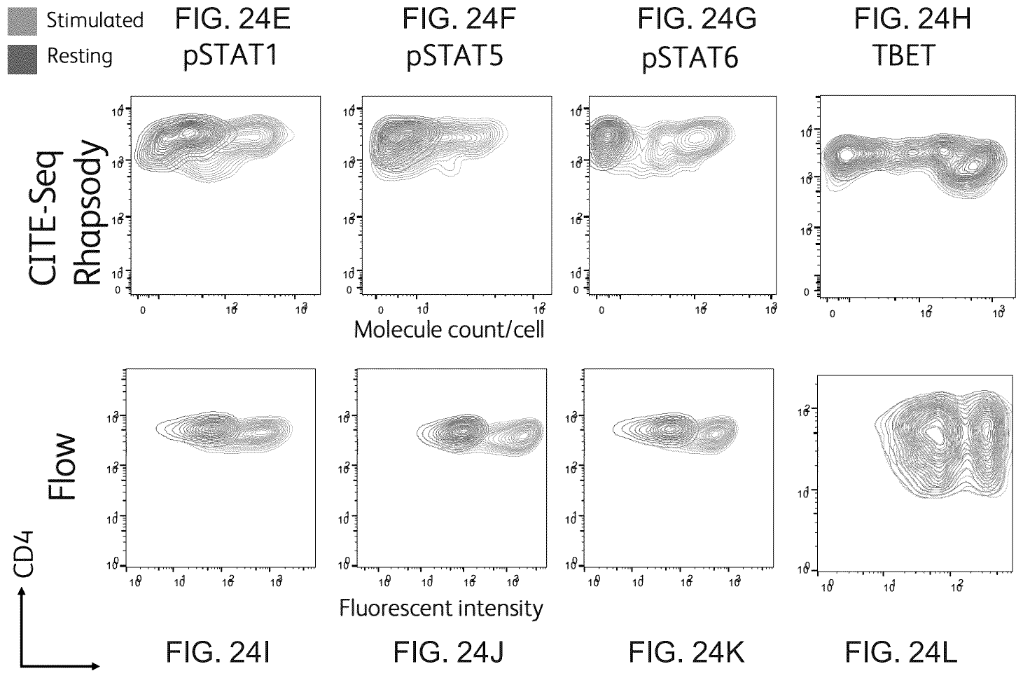

Simultaneous profiling of mRNA, surface and intracellular proteins was conducted. High mRNA and surface Abseq correlation +/−Intracellular Abseq is shown in FIG. 24A-24D. The detection of pSTAT1, pSTAT5 and pSTAT6 by intracellular CITE-seq is shown in FIG. 24E-24L. There is concordance between intracellular CITE-seq & Flow Cytometry. There was phosphorylation upon 15 min stimulation with hIFN-a, hIL-2, hIL-4 in CD4T cells.

### Example 11

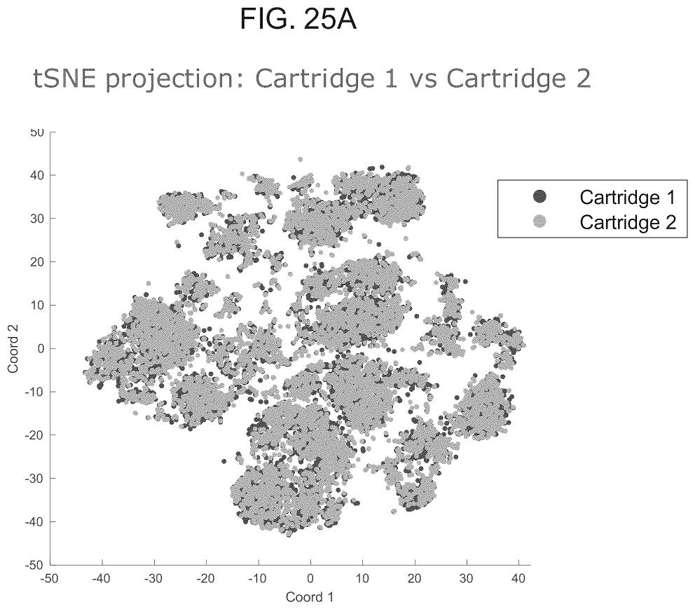

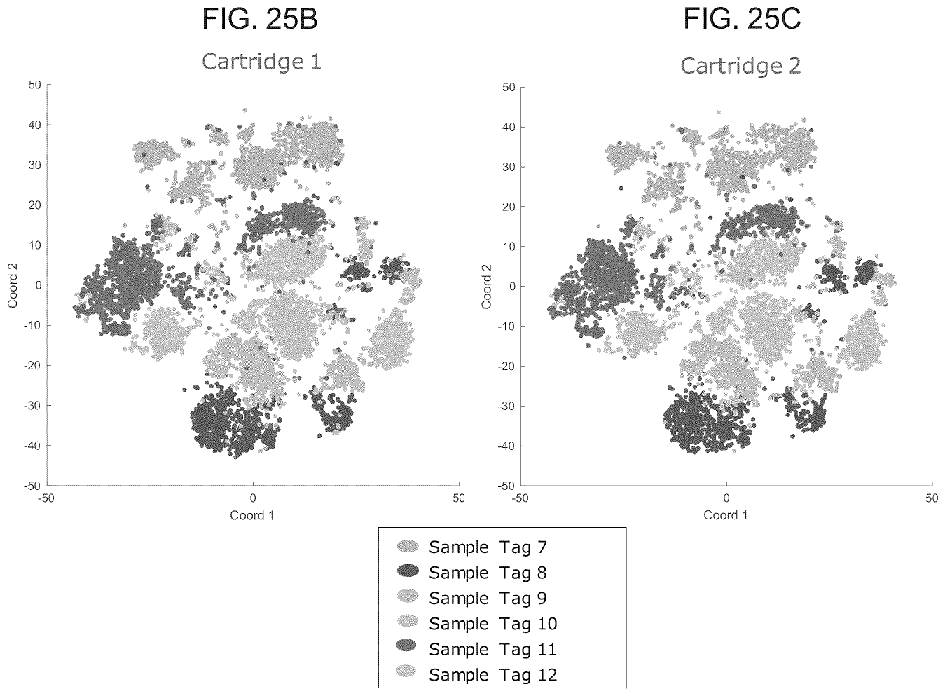

As shown in FIG. 25A-25C, there is no batch effect between technical replicates between two cartridges loaded with tagged cells. The markers split the sample into 6 distinct populations and results from the experiment show preservation of cell population independence for each cartridge and lane when overlaid on the t-SNE plot to example the sample tagged cell clusters.

Notwithstanding the appended claims, the disclosure is also defined by the following clauses:

1. A system comprising:
a tray configured to receive a multi-microwell-array-flowcell cartridge; and
a retrieval magnet assembly configured to apply a uniform magnetic force to flowcells of the cartridge when in an active position.

2. The system according to Clause 1, wherein the retrieval magnet assembly is configured to apply the uniform magnetic force from a position that is superior to the tray.

3. The system according to Clause 1 or 2, wherein the uniform magnetic force is a magnetic field ranging from 650 Gauss to 1325 Gauss.

4. The system according to any of the preceding clauses, wherein the retrieval magnet assembly is configured to apply the uniform magnetic force via a plurality of magnets.

5. The system according to Clause 4, wherein magnets of plurality have alternating polarities.

6. The system according to Clause 4 or 5, wherein the number of magnets in the retrieval magnet assembly ranges from 2 to 10.

7. The system according to Clause 6, wherein the retrieval magnet assembly comprises 4 magnets.

8. The system according to any of Clauses 4 to 7, wherein the plurality of magnets comprises rare earth magnets.

9. The system according to Clause 8, wherein the rare earth magnets are neodymium magnets.

10. The system according to Clause 8, wherein the rare earth magnets are samarium-cobalt magnets.

11. The system according to any of the preceding clauses, wherein magnets of the plurality are bar magnets.

12. The system according to any of Clauses 4 to 11, wherein magnets of the plurality are ring magnets.

13. The system according to Clause 12, wherein magnets of the plurality are arranged in a bullseye configuration.

14. The system according to any of the preceding clauses, wherein the retrieval magnet assembly is actuatable between the active position in which the retrieval magnet assembly is positioned adjacent to the tray, and an inactive position in which the retrieval magnet is located at a further distance from the tray relative to the active position.

15. The system according to any of the preceding clauses, further comprising a sample collection vessel holder configured to receive a plurality of sample collection vessels for collecting an analyte from the cartridge.

16. The system according to Clause 15, further comprising the plurality sample collection vessels.

17. The system according to Clause 16, wherein the system comprises a number of sample collection vessels ranging from 2 to 10.

18. The system according to Clause 16 or 17, wherein the system comprises a number of sample collection vessels that matches the number of flowcells in the cartridge.

19. The system according to Clause 17 or 18, wherein the system comprises 8 sample collection vessels.

20. The system according to any of Clauses 15 to 19, wherein the sample collection vessel holder comprises a counterweight configured to maintain the sample collection holder in an upright position.

21. The system according to any of Clauses 15 to 20, wherein system and the sample collection vessel holder have complementary shapes such that the sample collection vessel holder may be received in the system in a single orientation.

22. The system according to any of the preceding clauses, further comprising a drawer that is moveable between a plurality of different positions within the system.

23. The system according any of the preceding clauses, further comprising a waste collection vessel for collecting liquid waste from the multi-microwell-array-flowcell cartridge.

24. The system according to Clause 23, wherein the system comprises a single waste collection vessel.

25. The system according to any of Clauses 23 to 24, further comprising an interlock configured to prevent the collection of sample liquid into the waste collection vessel when the retrieval magnet assembly is in the active position.

26. The system according to any of the preceding clauses, wherein the tray comprises a latch for retaining the multi-microwell-array-flowcell cartridge.

27. The system according to any of the preceding clauses, wherein the system does not include a lysis magnet at an inferior position to the tray.

28. The system according to any of the preceding clauses, further comprising the multi-microwell-array-flowcell cartridge, wherein multi-microwell-array-flowcell cartridge comprises:
a plurality of fluidic lanes, each fluidic lane comprising:
   an inlet for receiving liquid;
   a flow cell comprising a microwell array; and
   an outlet for discharging liquid.

29. The system according to Clause 28, wherein the multi-microwell-array-flowcell cartridge comprises a number of fluidic lanes ranging from 2 to 10.

30. The system according to Clause 29, wherein the multi-microwell-array-flowcell cartridge comprises 8 fluidic lanes.

31. The system according to any of Clauses 28 to 30, wherein each outlet is stepped to prevent siphoning of liquid from the flow cell.

32. The system according to any of Clauses 28 to 31, wherein each flow cell is comprised of an elongate channel.

33. The system according to Clause 32, wherein the elongate channels range in length from 50 mm to 100 mm.

34. The system according to any of Clauses 28 to 33, wherein each microwell array comprises from 250,000 microwells to 300,000 microwells.

35. The system according to any of Clauses 28 to 34, wherein each microwell array comprises a density ranging from 36,000 microwells/cm² to 42,000 microwells/cm².

36. The system according to any of Clauses 28 to 35, wherein the tray has a shape that is complementary to the shape of the multi-microwell-array-flowcell cartridge such that the multi-microwell-array-flowcell cartridge may be received in the tray in a single orientation.

37. The system according to Clause 36, wherein the multi-microwell-array-flowcell cartridge comprises a chamfered corner.

38. The system according to any of the preceding clauses, further comprising a drip receptacle located at an inferior position to the tray configured to contain liquid discharged from the cartridge.

39. The system according to Clause 38, wherein the drip receptacle is detachable.

40. A method of processing a cellular sample, the method comprising:
introducing a multi-microwell-array-flowcell cartridge into a system comprising:
   a tray configured to receive the multi-microwell-array-flowcell cartridge; and
   a retrieval magnet assembly configured to apply a uniform magnetic force to the flowcells of the multi-microwell-array-flowcell cartridge when in an active position;
loading a sample into the multi-microwell-array-flowcell cartridge;
actuating the retrieval magnet assembly to the active position and applying the uniform magnetic force to the sample within the multi-microwell-array-flowcell cartridge to produce a processed sample; and
collecting the processed sample from the multi-microwell-array-flowcell cartridge.

41. The method according to Clause 40, wherein the method comprises loading a plurality of different samples into the different flowcells of the cartridge.

42. The method according to Clause 41, wherein number of different samples ranges from 2 to 10.

43. The method according to any of Clauses 40 to 42, further comprising loading a lysis buffer into the multi-well cartridge after the loading of the sample liquid into the multi-well cartridge.

44. The method according to Clause 43, where the method comprises loading the lysis buffer into the multi-well cartridge without engaging a lysis magnet.

45. The method according to any of Clauses 40 to 44, wherein the method comprises loading barcoded beads into the multi-well cartridge prior to the actuation of the retrieval magnet assembly.

46. The method according to Clause 45, wherein the barcoded beads comprise a nucleic acid barcode comprising a universal primer binding domain, a cell label domain and a target capture domain.

47. The method according to Clause 46, wherein the target capture domain is an poly(T) sequence.

48. The method according to Clause 46, wherein the nucleic acid barcode further comprises a unique molecular index (UMI).

49. The method according to any of Clauses 40 to 48, further comprising applying the uniform magnetic from a position that is superior to the tray.

50. The method according to any of Clauses 40 to 49, wherein the retrieval magnet assembly is configured to apply a uniform magnetic force to the flowcells of the multi-microwell-array-flowcell cartridge when in an active position via a plurality of magnets.

51. The method according to Clause 50, wherein magnets of the plurality have alternating polarities.

52. The method according to Clause 50 or 51, wherein the number of magnets in the retrieval magnet assembly ranges from 2 to 10.

53. The method according to Clause 52, wherein the retrieval magnet assembly comprises 4 magnets.

54. The method according to any of Clauses 50 to 53, wherein the plurality of magnets comprises rare earth magnets.

55. The method according to any of Clauses 40 to 54, wherein the system comprises a sample collection vessel holder configured to receive a plurality of sample collection vessels for collecting the analyte from the sample in the cartridge.

56. The method according to Clause 55, wherein the method comprises collecting the processed sample in a plurality of sample collection vessels.

57. The method according to Clause 56, wherein the method comprises collecting processed samples in a number of sample collection vessels ranging from 2 to 10.

58. The method according to Clause 56 or 57, wherein the method comprises collecting processed samples in a number of sample collection vessels that matches the number of flowcells in the cartridge.

59. The method according to Clause 57 or 58, wherein the method comprises collecting 8 processed samples in 8 sample collection vessels.

60. The method according to any of Clauses 55 to 59, wherein the sample collection vessel holder comprises a counterweight configured to maintain the sample collection holder in an upright position.

61. The method according to any of Clauses 40 to 60, wherein the system comprises a drawer that is moveable between a plurality of different positions within the system.

62. The method according to Clause 61, further comprising moving the drawer between the plurality of different positions within the system.

63. The method according to any of Clauses 40 to 62, further comprising collecting waste liquid from the multi-microwell-array-flowcell cartridge in a waste collection vessel.

64. The method according to any of Clauses 61 to 63, wherein the system comprises an interlock configured to prevent the collection of sample liquid into the waste collection vessel when the retrieval magnet assembly is in the active position.

65. The method according to Clause 62, further comprising engaging the interlock during the actuation of the retrieval magnet assembly.

66. The method according to any of Clauses 40 to 65, wherein the multi-microwell-array-flowcell cartridge comprises:
a plurality of fluidic lanes, each fluidic lane comprising:
   an inlet for receiving liquid;
   a flow cell comprising a microwell array; and
   an outlet for discharging liquid.

67. The method according to Clause 66, wherein the cartridge comprises a number of fluidic lanes ranging from 2 to 10.

68. The method according to Clause 67, wherein the cartridge comprises 8 fluidic lanes.

69. The method according to any of Clauses 66 to 68, wherein the outlet is stepped to prevent siphoning of liquid from the flow cell.

70. The method according to any of Clauses 66 to 69, wherein each flow cell is comprised of an elongate channel.

71. The method according to Clause 70, wherein the elongate channels range in length from 50 mm to 100 mm.

72. The method according to any of Clauses 66 to 71, wherein each microwell array comprises from 250,000 microwells to 300,000 microwells.

73. The method according to any of Clauses 66 to 72, wherein each microwell array comprises a density ranging from 36,000 microwells/cm² to 42,000 microwells/cm².

74. The method according to any of Clauses 40 to 73, wherein the method further comprises producing a sequence ready nucleic acid library from the processed sample.

75. The method according to Clause 74, wherein the sequence ready nucleic acid library is sequence-able by using a next generation sequencing protocol.

76. The method according to any of Clauses 40 to 75, wherein the method is a method of genomic analysis.

77. The method according to any of Clauses 40 to 75, wherein the method is a method of epigenomic analysis.

78. The method according to any of Clauses 40 to 75, wherein the method is a method of transcriptomic analysis.

79. The method according to any of Clauses 40 to 75, wherein the method is a method of proteomic analysis.

80. The method according to any of Clauses 40 to 75, wherein the method is a method of multiomic analysis.

81. The method according to Clause 80, wherein the multiomic analysis comprises at least transcriptomic and proteomic analysis.

82. A multi-microwell-array-flowcell cartridge comprising:
a plurality of fluidic lanes, each fluidic lane comprising:
   an inlet for receiving liquid;
   a flow cell comprising a microwell array; and
   an outlet for discharging liquid.

83. The multi-microwell-array-flowcell cartridge according to Clause 82, wherein the multi-microwell-array-flowcell cartridge comprises a number of fluidic lanes ranging from 2 to 10.

84. The multi-microwell-array-flowcell cartridge according to Clause 83, wherein the multi-microwell-array-flowcell cartridge comprises 8 fluidic lanes.

85. The multi-microwell-array-flowcell cartridge according to any of Clauses 82 to 84, wherein the outlet is stepped to prevent siphoning of liquid from the flow cell.

86. The multi-microwell-array-flowcell cartridge according to any of Clauses 82 to 85, wherein the outlet is a tapered conical orifice.

87. The multi-microwell-array-flowcell cartridge according to Clause 86, wherein the tapered conical orifice is configured to induce droplet formation for channel flow rates of 20 μL/s to 500 μL/s.

88. The multi-microwell-array-flowcell cartridge according to any of Clauses 82 to 87, wherein each flow cell is comprised of an elongate channel.

89. The multi-microwell-array-flowcell cartridge according to Clause 88 wherein the elongate channels range in length from 50 mm to 100 mm.

90. The multi-microwell-array-flowcell cartridge according to any of Clauses 82 to 89, wherein each microwell array comprises from 250,000 microwells to 300,000 microwells.

91. The multi-microwell-array-flowcell cartridge according to any of Clauses 82 to 90, wherein each microwell array comprises a density ranging from 36,000 microwells/cm² to 42,000 microwells/cm².

92. The multi-microwell-array-flowcell cartridge according to any of Clauses 82 to 91, wherein the multi-microwell-array-flowcell cartridge comprises a chamfered corner.

93. The multi-microwell-array-flowcell cartridge according to any of Clauses 82 to 92, wherein each inlet is comprised of a gasket.

94. The multi-microwell-array-flowcell cartridge according to Clause 93, wherein the gasket comprises a Shore durometer ranging from 10 to 80.

95. The multi-microwell-array-flowcell cartridge according to Clause 93, wherein the gasket is configured to taper lock on a pipette tip.

96. The multi-microwell-array-flowcell cartridge according to Clause 95, wherein the taper lock has a Z-axis tolerance of 0.5 mm.

97. A sample vessel holder configured to receive a plurality of sample collection vessels for collecting an analyte from the multi-microwell-array-flowcell cartridge according to any of Clauses 82 to 96.

98. The sample vessel holder according to Clause 97, wherein the sample vessel holder is configured to receive a number of sample collection vessels ranging from 2 to 10.

99. The sample vessel holder according to Clause 98, wherein the sample vessel holder is configured to receive 8 sample collection vessels.

100. The sample vessel holder according to any of Clauses 97 to 99, wherein the sample collection vessel holder comprises a counterweight configured to maintain the sample collection holder in an upright position.

101. The sample vessel holder according to any of Clauses 97 to 100, wherein the sample collection vessel holder has a complementary shape to a cellular analysis system such that the sample collection vessel holder may be received in the cellular analysis system in a single orientation.

102. A kit comprising:
a multi-microwell-array-flowcell cartridge comprising:
   a plurality of fluidic lanes, each fluidic lane comprising:
      an inlet for receiving liquid;
      a flow cell comprising a microwell array; and
      an outlet for discharging liquid.

103. The kit according to Clause 102, wherein the cartridge comprises a number of fluidic lanes ranging from 2 to 10.

104. The kit according to Clause 103, wherein the cartridge comprises 8 fluidic lanes.

105. The kit according to any of Clauses 102 to 104, wherein each outlet is stepped to prevent siphoning of liquid from the flow cell.

106. The kit according to any of Clauses 102 to 105, wherein the outlet is a tapered conical orifice.

107. The kit according to Clause 106, wherein the tapered conical orifice is configured to induce droplet formation for channel flow rates of 20 μL/s to 500 μL/s.

108. The kit according to any of Clauses 102 to 107, wherein each flow cell is comprised of an elongate channel.

109. The kit according to Clause 108, wherein the elongate channels range in length from 50 mm to 100 mm.

110. The kit according to any of Clauses 102 to 109, wherein each microwell array comprises from 250,000 microwells to 300,000 microwells.

111. The kit according to any of Clauses 102 to 110, wherein each microwell array comprises a density ranging from 36,000 microwells/cm² to 42,000 microwells/cm².

112. The kit according to any of Clauses 102 to 111, wherein the multi-microwell-array-flowcell cartridge comprises a chamfered corner.

113. The kit according to any of Clauses 102 to 112, wherein each inlet is comprised of a gasket.

114. The kit according to Clause 113, wherein the gasket comprises a Shore durometer ranging from 10 to 80.

115. The kit according to Clause 113, wherein the gasket is configured to taper lock on a pipette tip.

116. The kit according to Clause 113, wherein the taper lock has a Z-axis tolerance of 0.5 mm.

117. The kit according to any of Clauses 102 to 116, wherein the kit comprises a plurality of multi-microwell-array-flowcell cartridges.

118. The kit according to any of Clauses 102 to 117, further comprising a sample collection vessel holder configured to receive a plurality of sample collection vessels.

119. The kit according to Clause 118, wherein the sample collection vessel holder comprises a counterweight configured to maintain the sample collection vessel holder in an upright position.

120. The kit according to any of Clauses 102 to 119, further comprising a plurality of sample collection vessels.

121. The kit according to any of Clauses 102 to 120, further comprising a waste collection vessel.

122. The kit according to any of Clauses 102 to 121, further comprising a cell lysis buffer.

123. The kit according to any of Clauses 102 to 122, further comprising a hybridization buffer.

124. The kit according to any of Clauses 102 to 123, further comprising a wash buffer.

125. The kit according to any of Clauses 102 to 124, further comprising a reducing agent.

126. The kit according to any of Clauses 102 to 125, further comprising barcoded beads.

127. The kit according to Clause 126, wherein the barcoded beads comprise nucleic acid barcodes comprising a universal primer binding domain, a cell label domain and a target capture domain.

128. The kit according to Clause 127, wherein the target capture domain is an oligo dT sequence.

129. The kit according to Clause 127, wherein the nucleic acid barcode further comprises a unique molecular index (UMI).

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. Moreover, nothing disclosed herein is intended to be dedicated to the public regardless of whether such disclosure is explicitly recited in the claims.

The scope of the present invention, therefore, is not intended to be limited to the exemplary embodiments shown and described herein. Rather, the scope and spirit of present invention is embodied by the appended claims. In the claims, 35 U.S.C. § 112(f) or 35 U.S.C. § 112(6) is expressly defined as being invoked for a limitation in the claim only when the exact phrase “means for” or the exact phrase “step for” is recited at the beginning of such limitation in the claim; if such exact phrase is not used in a limitation in the claim, then 35 U.S.C. § 112 (f) or 35 U.S.C. § 112(6) is not invoked.

1. A system comprising:
a tray configured to receive a multi-microwell-array-flowcell cartridge; and
a retrieval magnet assembly configured to apply a uniform magnetic force to flowcells of the cartridge when in an active position.

2. The system according to claim 1, wherein the retrieval magnet assembly is configured to apply the uniform magnetic force from a position that is superior to the tray.

3. (canceled)

4. The system according to claim 1, wherein the retrieval magnet assembly is configured to apply the uniform magnetic force via a plurality of magnets.

5. The system according to claim 4, wherein magnets of plurality have alternating polarities.

6-13. (canceled)

14. The system according to claim 1, wherein the retrieval magnet assembly is actuatable between the active position in which the retrieval magnet assembly is positioned adjacent to the tray, and an inactive position in which the retrieval magnet is located at a further distance from the tray relative to the active position.

15. The system according to claim 1, further comprising a sample collection vessel holder configured to receive a plurality of sample collection vessels for collecting an analyte from the cartridge.

16. The system according to claim 15, further comprising the plurality sample collection vessels.

17. (canceled)

18. The system according to claim 16, wherein the system comprises a number of sample collection vessels that matches the number of flowcells in the cartridge.

19. (canceled)

20. The system according to claim 15, wherein the sample collection vessel holder comprises a counterweight configured to maintain the sample collection holder in an upright position.

21. The system according to claim 1, wherein system and the sample collection vessel holder have complementary shapes such that the sample collection vessel holder may be received in the system in a single orientation.

22. The system according to claim 1, further comprising a drawer that is moveable between a plurality of different positions within the system.

23. The system according to claim 1, further comprising a waste collection vessel for collecting liquid waste from the multi-microwell-array-flowcell cartridge.

24. The system according to claim 23, wherein the system comprises a single waste collection vessel.

25. The system according to claim 23, further comprising an interlock configured to prevent the collection of sample liquid into the waste collection vessel when the retrieval magnet assembly is in the active position.

26. The system according to claim 1, wherein the tray comprises a latch for retaining the multi-microwell-array-flowcell cartridge.

27. The system according to claim 1, wherein the system does not include a lysis magnet at an inferior position to the tray.

28. The system according to claim 1, further comprising the multi-microwell-array-flowcell cartridge, wherein multi-microwell-array-flowcell cartridge comprises:
a plurality of fluidic lanes, each fluidic lane comprising:
an inlet for receiving liquid;
a flow cell comprising a microwell array; and
an outlet for discharging liquid.

29-30. (canceled)

31. The system according to claim 28, wherein each outlet is stepped to prevent siphoning of liquid from the flow cell.

32. The system according to claim 28, wherein each flow cell is comprised of an elongate channel.

33-35. (canceled)

36. The system according to claim 28, wherein the tray has a shape that is complementary to the shape of the multi-microwell-array-flowcell cartridge such that the multi-microwell-array-flowcell cartridge may be received in the tray in a single orientation.

37-129. (canceled)
